(19) United States
(12) Patent Application Publication
(10) Pub. No.: US 2013/0245604 A1
(43) Pub. Date: Sep. 19, 2013

(54) Auto-Injector Device with a Medicated Module

(71) Applicants: Garen Kouyoumjian, Warwickshire (GB); Malcolm Stanley Boyd, Warwickshire (GB); Daniel Thomas De Sausmarez Lintell, Warwickshire (GB)

(72) Inventors: Garen Kouyoumjian, Warwickshire (GB); Malcolm Stanley Boyd, Warwickshire (GB); Daniel Thomas De Sausmarez Lintell, Warwickshire (GB)

(73) Assignee: SANOFI-AVENTIS DEUTSCHLAND GMBH, Frankfurt am Main (DE)

(21) Appl. No.: 13/990,110

(22) PCT Filed: Nov. 28, 2011
(86) PCT No.: PCT/EP11/71135

§ 371 Date: May 29, 2013

Related U.S. Application Data
(60) Provisional application No. 61/433,830, filed on Jan. 18, 2011.

(30) Foreign Application Priority Data
Nov. 29, 2010 (EP) 10192993.3

(51) Int. Cl. A61M 5/20

(52) U.S. Cl. CPC A61M 5/2066
USPC 604/506; 604/131

(57) ABSTRACT

Disclosed herein are various examples of a drug delivery system and corresponding method for delivering three or more medicaments. The system includes two major components: an auto-injector device that contains at least two medicaments and a medicated module that contains at least one medicament. The medicated module interfaces with the auto-injector device such that a combination dose comprising all of the medicaments can be delivered via a single dispense interface of the medicated module. In order to deliver a pre-defined combination dose, a user need only set the dose of one of the medicaments contained in the auto-injector device and need only activate the system once by actuating a dose delivery button on the auto-injector device.

### FIELD OF THE PRESENT PATENT APPLICATION

The present patent application relates to medical devices and methods of delivering multiple fluids and/or medicaments using a device having a single dose setting mechanism and a single dispense interface. The fluids and/or medicaments may be contained in one or more cartridges, reservoirs, containers or packages, each containing independent (single compound) or pre-mixed (co-formulated multiple compounds) drug agents. The disclosed device is of particular benefit where combination therapy is desirable, but not possible in a single formulation for reasons such as, but not limited to, stability, compromised therapeutic performance and toxicology.

### BACKGROUND

Certain disease states require and/or benefit from treatment using two or more different medicaments (i.e., combination therapy). For example, in some cases it might be beneficial to treat a diabetic with a long acting insulin (also may be referred to as the first or primary medicament) along with a glucagon-like peptide-1 such as GLP-1 or GLP-1 analog (also may be referred to as the second drug or secondary medicament). GLP-1 is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

Although certain disease states require and/or benefit from combination therapy, there are a number of potential problems associated with delivering two active medicaments or “drug agents” simultaneously. For instance, certain medicaments need to be delivered in a specific relationship with each other in order to deliver the optimum therapeutic dose. Additionally, the two active drug agents may interact with each other during the long-term shelf-life storage of the formulation. Therefore, it is advantageous to store the active drug agents separately and only combine them at the point of delivery, for example, by injection, needle-less injection, pumps, or inhalation. However, the process for combining the two agents and then administering this combination therapy needs to be simple and convenient for the user to perform reliably, repeatedly and safely.

A further problem that may arise is that the quantities and/or proportions of each active drug agent making up the combination therapy may need to be varied for each user or at different stages of their therapy. For example, one or more active drug agents may require a titration period to gradually introduce a patient to a “maintenance” dose. A further example would be if one active drug agent requires a non-adjustable fixed dose while the other active agent is varied. This other active agent may need to be varied in response to a patient's symptoms or physical condition. Therefore, certain pre-mixed formulations comprising two or more active drug agents may not be suitable as these pre-mixed formulations would have a fixed ratio of the active components, which could not be varied by the healthcare professional or user.

Additional problems can arise where a combination therapy is required because many users cannot cope with having to use more than one drug delivery system or make the necessary accurate calculation of the required dose combination. Other problems arise where a drug delivery system requires the user to physically manipulate the drug delivery device or a component of the drug delivery device (e.g., a dose dialing button) so as to set and/or inject a dose. This may be especially true for certain users who are challenged with dexterity or computational difficulties.

In light of the above-mentioned problems, there exists a need to provide devices and/or methods for the delivery of multiple medicaments that require only a single dose setting step and a single injection or delivery step that is simple for the user to perform without complicated physical manipulations of the drug delivery device.

### SUMMARY

Disclosed herein are various examples of a drug delivery system and corresponding method for delivering (herein, sometimes referred to as “dispensing”) three or more fluids and/or medicaments, where each medicament contains independent (single compound) or pre-mixed (co-formulated multiple compounds) drug agents. As disclosed herein, the system includes two major components: an auto-injector device that contains at least two medicaments and a medicated module that contains at least one medicament. The medicated module interfaces with the auto-injector device such that a combination dose comprising all of the medicaments can be delivered via a single dispense interface (e.g., a needle cannula) of the medicated module. Although principally described in this application as an injection drug delivery system, the basic principle could be applicable to other forms of drug delivery, such as, but not limited to, inhalation, nasal, ophthalmic, oral, topical, and like devices.

The disclosed system and corresponding method allow a user to set doses of the medicaments contained within the auto-injector via a single dose setting mechanism of the auto-injector device. The single dose setting mechanism of the auto-injector may include a dose setter that comprises a digital display, a soft-touch operable panel, and/or graphical user interface (GUI). The single dose setting mechanism allows a predefined combination of drug agents within the auto-injector to be set (based in part on a selected therapeutic dose algorithm that may either be previously selected prior to dose setting or at the time that the dose is set) when a single dose of one of the medicaments in the auto-injector is set. Further, the user need not take any dose-setting action with respect to the medicament in the medicated module because when the medicated module is attached to the auto-injector device, the single dose of medicament within the medicated module is essentially set. Therefore, after setting a dose of one of the medicaments within the auto-injector, the combination dose (including the dose of medicament in the medicated module) can be dispensed through the single dispense interface of the medicated module by a single activation of the system (e.g., actuating a dispense button of the auto-injector). When the user activates the device, the medicaments that flow from the auto-injector device and through the medicated module force the fixed dose of medicament out of the medicated module.

In one example, the drug delivery system comprises (a) an auto-injector device that includes (i) a dose setting mechanism, (ii) a first cartridge containing a first medicament, (iii) a second cartridge containing a second medicament, (iv) an interface hub including an outlet port that is in fluid communication with the first and second cartridges, and (v) a delivery button, and (b) a medicated module attached to the interface hub of the auto-injector device, where the medicated module includes (i) a reservoir containing a third medicament, (ii) a proximal needle, (iii) a distal needle, and (iv) a slidable needle guard. A pre-defined amount of proximal movement of the needle guard places the distal needle in fluid communication with the first and second medicaments contained in the auto-injector drug delivery device and in fluid communication with the third medicament contained in the medicated module. A single actuation of the delivery button of the auto-injector device causes a combination dose of the first, second, and third medicaments to be delivered via the distal needle of the medicated module. During delivery, the first and second medicaments flow through the reservoir of the medicated module, thereby forcing the third medicament out of the reservoir. The interface hub may comprise a first and a second proximal needle, where the first and second proximal needles are in fluid communication with the first and second cartridges respectively.

In one example described herein, the auto-injector includes an electro-mechanical dose setting mechanism by which a desired therapeutic dose profile of the at least two medicaments contained therein may be achieved using a microprocessor that is programmed to control, define, and/or optimize a therapeutic dose profile. A plurality of potential dose profiles may be stored in memory coupled to the microprocessor. For example, such stored therapeutic dose profiles may include, but are not limited to, a linear dose profile; a non-linear dose profile; a fixed ratio-fixed dose profile; a fixed dose-variable dose profile; a delayed fixed dose-variable dose profile; or a multi-level, fixed dose variable dose profile as discussed and described in greater detail below. Alternatively, only one dose profile would be stored in a memory device operatively coupled to the microprocessor. These dose profiles refer to the two or more medicaments contained in the auto-injector device.

Upon setting a dose of the first or primary medicament in the auto-injector device, the micro-processor automatically calculates the dose of a second medicament (i.e., non-user settable) in the auto-injector device based on a programmed therapeutic dose profile or programmed algorithm. In an alternative arrangement, the auto-injector may contain more than two medicaments and upon setting the dose of the first medicament, the micro-processor may automatically calculate the dose of a second medicament and a third medicament based on a programmed therapeutic dose profile or programmed algorithm. The profile used to compute the dose of the third medicament may or may not be the same type of profile used to compute the dose of the secondary medicament. Regardless of the dose profile of the medicaments contained in the auto-injector device, the dose of the medicament contained in the medicated module is not settable by the user, rather, it is fixed and primarily based on the size of the medicament module reservoir.

The quantity of medicaments used with Applicants' drug delivery system may vary. For example, one fluid quantity can be varied by changing the properties of the auto-injector device (e.g., setting a user variable dose or changing the device's “fixed” dose). The second, third, forth, etc. medicament quantities can be changed by manufacturing a variety of secondary drug containing reservoirs and/or medicament modules with each variant containing a different volume and/or concentration of the second, third, fourth, etc. medicament. The user (e.g., a patient, a healthcare professional or any other person using the device) would then select the most appropriate secondary package, medicament module, or series or combination of series of different packages/modules for a particular treatment regime.

By defining the therapeutic relationship between the medicaments, the proposed system helps to ensure that a patient/user receives the optimum therapeutic combination dose. This combination dose may be set and administered without the inherent risks that may be associated with multiple inputs, where the user is often called upon to calculate and set the correct dose combination each time that the device is used to administer a dose. The medicaments can be fluids, defined herein as liquids, gases or powders that are capable of flowing and that change shape when acted upon by a force tending to change its shape. Alternatively, one of the medicaments may be a solid where such a solid may be carried, solubilized or otherwise dispensed with another fluid, for example a fluid medicament or a liquid. In one example, a master drug compound, such as insulin, contained within the auto-injector device could be used with at least a secondary medicament contained within the same device and a third medicament contained within the medicated module.

The proposed drug delivery system is of particular benefit to users with dexterity or computational difficulties as the single dose setting action removes the need for a user to calculate a prescribed dose every time they use the device. In addition, the single input allows easier dose setting and dose administration of the combined compounds. The electro-mechanical nature of the system also benefits users with dexterity and visual challenges since it may be operated and/or controlled by way of a micro-processor based operator panel.

In one example, the auto-injector device comprises a main body comprising a microprocessor based control unit. An electro-mechanical drive unit is operably coupled to the control unit. The electro-mechanical drive unit is coupled to a primary reservoir and a secondary reservoir. Preferably, the electro-mechanical drive unit is coupled to the primary reservoir and the secondary reservoir by way of a first and a second drive train. The first and the second drive trains may be similar in operation. An operator interface is in communication with the control unit.

A medicated module that includes a dispense interface may be configured for fluid communication (either directly or via an intermediate component, e.g., an interface hub) with the primary and the secondary reservoirs. Activation of the operator panel sets a dose of the primary medicament within the primary reservoir. Based on at least the selected dose of the primary medicament, the control unit computes a dose of the secondary medicament contained within the auto-injector, based at least in part on a therapeutic dose profile. In an alternative arrangement, based on at least the selected dose of the primary medicament, the control unit computes a dose range of the secondary medicament based at least in part on a therapeutic dose profile. A user may then select a dose of the secondary medicament within the determined range. Based on at least the selected dose of the primary medicament, the control unit may also compute a dose or a dose range of an additional medicament contained in the auto-injector based at least in part on a therapeutic dose profile. During delivery, the primary medicament may or may not be administered to an injection site simultaneously with the secondary medicament.

In one arrangement, the selected profile may be determined when a cartridge of medicament is inserted into a cartridge retainer of the auto-injector device. A cartridge may comprise one or more reservoirs for storing and releasing one or more medicaments. Separate cartridges for each medicament may be used, or a single cartridge with multiple reservoirs may be used. For example, the cartridge retainer of the auto-injector device may contain a cartridge identification circuit that when or if the device ‘reads’ a cartridge identifier provided on the inserted cartridge, logic contained in the device could determine which of the plurality of stored profiles is the appropriate profile to select for the particular medicament contained within the cartridge. In one such arrangement, this selection process might therefore be fully automatic. That is, no user intervention is required to select the proper profile. In an alternative embodiment, cartridge identification information may be used to request a profile through a wired or wireless connection, for example a universal serial bus (USB) connection, a Bluetooth™ connection, a cellular connection and/or the like. The profile may be requested from an internet page. The profile may be received by the device through the same wired or wireless connection. The profile may then be stored and applied in the apparatus without any user intervention or after confirmation by a user.

Alternatively, this therapeutic profile selection process might be semi-automatic. For example, this therapeutic profile may be suggested and selected via a graphical user interface provided on a digital display. For example, the GUI may prompt the user to confirm which profile they want from a limited range of options or fully configurable by the user, for example by a patient or health care provider.

Although the present application specifically mentions insulin, insulin analogs or insulin derivatives, and GLP-1 or GLP-1 analogs as two possible drug combinations, other drugs or drug combinations, such as an analgesics, hormones, beta agonists or corticosteroids, or a combination of any of the above-mentioned drugs could be used with our invention.

For the purposes of the present application, the term “insulin” shall mean Insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

As used herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin. By user settable dose it is meant that the user can select the desired dose. For example, as noted above, the user can select a dose of the primary medicament contained in the auto-injector device. The user settable dose may be set remotely through a communications port such as a wireless communication port (e.g., Bluetooth, WiFi, satellite, etc.). Alternatively, the user settable dose can be set through a wired communications port such as a Universal Serial Bus (USB) communications port. Additionally, the dose may be set by another device, such as a blood glucose monitor after performing a therapeutic treatment algorithm.

By calculated dose, it is meant that the user (or any other input) cannot independently set or select a dose of medicament. For instance, as noted above in one example, the secondary medicament in the auto-injector device cannot be set by the user, rather it is computed by the device to achieve a predefined therapeutic profile of a combination of both primary and secondary medicaments. In other words, when the user (or another input as described above) sets the dose of the primary medicament in the primary reservoir of the auto-injector device, the dose of the second medicament contained in the auto-injector is determined by the microprocessor control unit.

By fixed dose, it is meant that the user cannot independently set or select a dose of medicament. For example, the dose of the medicament contained in the medicated module is fixed the moment the medicated module is attached to the auto-injector.

The combination of medicaments may be delivered to the user as discrete units or as a mixed unit via the dispense interface of the medicated module. Thus providing a combination drug injection system that, from the user's perspective, is achieved in a manner that closely matches the currently available injection devices that use standard needle assemblies. One possible delivery procedure may involve the following steps:
1. Attach an interface hub to a distal end of an electro-mechanical auto-injector device. The first and second needles of the interface pierce a first reservoir containing a primary medicament and a second reservoir containing a secondary medicament, respectively.
2. Attach a medicated module that contains a third medicament and that has a proximal and distal needle (i.e., dispense interface) to a distal end of the interface such that the proximal needle of the medicated module is in fluid communication with both the primary and secondary medicaments.
3. Set a desired dose of the primary medicament using the dose setter of the auto-injector device (e.g., a graphical user interface (GUI)).
4. After the user sets the dose of the primary medicament, the micro-processor controlled control unit determines or computes a dose of the secondary medicament and preferably determines or computes this second dose based on a previously stored therapeutic dose profile. It is this computed combination of medicaments that will then be injected along with the third medicament in the medicated module.
5. Optionally, after the second dose has been computed, the auto-injector device may be placed in an armed condition. Such an optional armed condition may be achieved by pressing and/or holding an “OK” button on a control panel. This condition may provide for greater than a predefined period of time before the device can be used to dispense the combined dose.
6. The needle guard of the medicated module can then be pressed against the skin of the user such that the needle guard retracts, thereby placing the distal needle of the medicated module in fluid communication with all three medicaments. This action also causes the distal needle to enter the injection site. The combination dose of the three medicaments are then administered by activating an injection user interface (e.g., an injection button) on the auto-injector.

The proposed drug delivery system may be designed in such a way as to limit its use to exclusive primary and secondary reservoirs, as well as exclusive medicated modules, through employment of dedicated or coded features. This would help to prohibit the use of incorrect medicaments.

A particular benefit of the proposed drug delivery system is that the use of two or more multi-dose reservoirs in the auto-injector device, along with the single dose reservoir in the medicated module, makes it possible to tailor dose regimes when required, for example where a titration period is necessary for a particular drug. For instance, the secondary reservoir and/or medicated module may be supplied in a number of titration levels with certain differentiation features such as, but not limited to, aesthetic design of features or graphics, numbering or the like symbols, so that a user could be instructed to use the supplied secondary reservoirs and/or medicated modules in a specific order to facilitate titration. Alternatively, a prescribing physician or health care provider may provide the patient with a number of “level one” titration secondary reservoirs and/or medicated modules and then when these were finished, the physician could then prescribe the next level. Alternatively, a single strength formulation could be provided and the device could be designed to deliver a pre-defined fraction of the full intended dose during the titration period. Such a fraction could be gradually increased, stepped, etc. One advantage of such a titration program is that the primary device remains constant throughout the administration process.

In one embodiment, the drug delivery system is used more than once and therefore is multi-use. Such a system may or may not have replaceable reservoirs for the primary and secondary medicaments. However, because the medicated module is intended for a single use, it would need to be replaced after delivering each combination dose. It is possible to have a suite of different secondary reservoirs and medicated modules for various conditions that could be prescribed as one-off extra medication to patients.

In one embodiment of the system, the medicated module comprises an outer housing having a proximal end, a distal end, and an outer surface, where the proximal end preferably has a hub holding a double-ended needle and is configured for attachment (either directly or indirectly via an intermediate component) to the auto-injector device. The double ended needle is positioned such that is placed in fluid communication with the reservoirs of the auto-injector when the medicated module is attached to the auto-injector device. There is a reservoir in a bypass housing within the outer housing that contains a medicament. The medicated module further includes a needle guard that can reduce the risk of accidental needle sticks before and after use, reduce the anxiety of users suffering from needle phobia as well as preventing a user from using the device a subsequent time when the medicament has already been expelled.

The needle guard is preferably configured with a solid planar surface at its distal end that provides a large surface area that reduces the pressure exerted on the user's skin, which allows the user to experience an apparent reduction in the force exerted against their skin. The planar surface may cover the entire distal end of the guard with the exception of a small needle pass through hole aligned axially with the distal needle (i.e., the dispense interface). This pass through hole is preferably no more than 10 times greater in diameter than the outer diameter of the distal needle. For example, with a needle outside diameter of 0.34 mm, the pass through hole diameter D may be 4 mm. Preferably, the pass through hole size should be large enough for the user to see that the device is primed (i.e., a drop or more of medicament) while not being so large that it is still possible to reach the end of the needle with a finger (i.e. needle stick injuries before or after use). This particular ratio between the hole size and the needle diameter helps accommodate tolerances of the various medicated module components and also allows users to see a drop of liquid on the end of the needle after priming (whether a transparent or non-transparent guard is used) while keeping the size small enough to prevent accidental needle stick injuries.

Further, the movable needle guard or shield is configured to move axially in both the distal and proximal directions when pressed against and removed from an injection site. When the distal needle is withdrawn from the patient, the guard is returned to its post-use extended position. A drive tooth on the inside surface of the guard engages a stop on a track on the outer surface of the bypass housing to securely lock the guard from further substantial axial movement. Preferably, a lock out boss on the outer surface of the bypass housing is configured to engage a lock out feature on the inner proximal surface of the outer housing at the completion of the injection to further lock the medicated module from any further use and prevent the needle(s) and/or bypass component from being able to substantially move within the system even if the guard is held in an axially locked condition. By “substantial” movement we do not mean the typical amount of “play” in a system, but instead we mean that the guard and/or distal needle do not move axially a distance that exposes the distal end of the needle once it is locked out.

The medicated module is configured to change from a priming state to a combination dose delivery state without manual operation by the user, which is beneficial because manually operated devices are sometimes not as intuitive and can raise the risk of accidental misuse. The medicated module described herein eliminates the need for manual operation by the user by utilizing energy stored within the module prior to delivery of the device to the user. The stored energy can come from a biasing member, such as a compressed spring. This stored energy is released during normal user operation of the module by actuating the mechanism and thus causing the medicated module to change from a dose priming state to a combination dose state. The mechanism aims to make this actuation imperceptible to the user, consequently making the user experience of the module very similar to that of a standard commercially available and accepted needle or safety needle (i.e. unpack module, attach to a drug delivery device, prime drug delivery device, inject a set dose along with single dose in the module). In this way, the module mechanism aims to reduce the risk of unintentional misuse and to improve usability by replicating an already accepted practice for similar injection methods. Once, the medicated module is in a combination dose delivery state, retraction of the needle guard as it is pressed against the skin of the user causes the spring to store additional energy which is used after the needle is withdrawn from the injection site in order to force the needle guard in the distal direction to its lock-out position.

Retraction of the needle guard causes the spring to store additional energy. For this mechanism to work it is irrelevant of what makes the needle guard retract, e.g. the needle guard could be pulled back, pushed back, pushed against any surface. However, in the field of drug delivery devices it may be beneficial when the needle guard retracts as it is pressed against the skin of the user. This improves user comfort as well as user safety.

Once the needle guard is free to move the additional stored energy forces the needle guard in the distal direction. For the mechanism to work it is essential that the needle guard is free to move axially, e.g. nothing holds or fixes the needles guard with regards to its axial position. However, in the area of drug delivery device the needle guard may be free to move axially after the needle is withdrawn from the injection site and the needle guard may be forced in the distal direction.

As the module mechanism does not require the user to access external features on the module during priming, dosing, or after dosing to place the medicated module in its lockout position, the number of components and subsequent module size can be reduced/optimized. These factors make the mechanism ideal for a single-use, high-volume manufacture, and disposable device application. However, the medicated module may be designed to be resettable. The preferred embodiment described below is the single use (non-resettable) version. The lower hub is preferably restrained rotationally with regard to the needle guard, but is free to move axially within the needle guard. The needle guard is restrained rotationally with regard to the outer housing, but is free to move axially, between defined constraints, within the outer housing. When the user presses the distal face of the needle guard against their skin the needle guard moves in the proximal direction. This proximal axial motion of the guard causes a rotation of the bypass housing through the engagement and action of an inward-facing drive tooth on the guard as it travels in a drive track having one or more paths, which is located on the outer surface of the bypass housing. After sufficient axial travel of the needle guard, the rotation of the bypass housing brings stand-offs inside the outer housing and at the proximal ends of the lower hub into line with pockets located on the outer surface of the bypass housing. Alignment of the stand-offs with the pockets allows the bypass housing to move axially in the proximal direction and further into the outer housing. The lower hub containing a double-ended needle cannula moves axially further onto the bypass housing. It is this axial movement of the lower hub onto the bypass housing and the corresponding movement of the bypass housing further into the outer body that results in the double ended needles located in the outer body distal end and the lower hub piercing the medicated module, moving it from a state of priming to a state of combination dose delivery.

Further axial movement of the needle guard is required in order to pierce the skin, this retraction of the needle guard temporarily re-compresses the biasing member creating additional stored energy. At a “commit” point, the proximal axial movement of the drive tooth passes a non-return feature in the track through further rotation of the bypass housing. In normal use, once the medicament has been dispensed and the needle is removed from the skin, the needle guard is allowed to return axially in the distal direction under the relaxation of the biasing member as it releases its stored energy. At some point along its return travel, the drive tooth contacts a further ramped face in one of the paths of the track, resulting in yet further rotation of the bypass housing. At this point, the outer housing stand-off comes into contact with a ramp feature on the outer surface of the bypass housing. The combination of this feature with the ramp between the drive tooth and the bypass housing track results in further biasing of the bypass housing stop face into the needle guard drive tooth. The stop face features act as an axial locking pocket. The action of the combined biasing force means that any axial load in the proximal direction put on the needle guard will result in the tooth being stopped in this pocket, locking out the needle guard from further use or exposing the needle. Should the user remove the device from the skin without dispensing fluid, but after the “commit” point has been passed, the needle guard would return to an extended position and lock out as previously described.

The proximal hub of the medicated module can be a separate part from the housing or integral to the housing. For example, the hub may be molded as part of the housing. The connector mechanism that connects the medicated module to the auto-injector device can be any connector mechanism, such as threads, snap fits, bayonet, lure lock, or combination of these designs.

Two needle cannula are used in the medicated module, a distal cannula and a proximal cannula, with both cannulae preferably being doubled-ended and capable of piercing a septum or seal and for piercing skin. The distal needle is mounted in a lower hub and the proximal needle is mounted in the upper hub, each using any technique known to those skilled in the art, such as welding, gluing, friction fit, over-molding and the like. As noted above, the medicated module assembly also contains a biasing member, preferably a compression spring. The biasing member is preferably in a pre-compressed state and positioned between the proximal inner face of the needle guard and the distal face of the lower hub. Although a preferred biasing member is a spring, any type of member that produces a biasing force will work.

As noted above, the medicated module assembly of our invention automatically, once triggered, changes state from (1) a pre-use or priming state, where a small amount of primary and secondary medicament flows from the auto-injector and through a bypass around the reservoir containing a single dose of a third medicament, to (2) a ready-to-use or combination dose state, where both the upper and lower cannulae are in fluid engagement with the fixed dose of the third medicament within the module and where set doses of the primary and secondary medicaments can be injected along with the non-settable single dose of the third medicament in the reservoir, and finally to (3) a locked out state, where the needle guard is prevented from substantial proximal movement. The outer housing of the medicate module preferably has a window or indicator that shows the various states of the module. The indicator can be a pip, knob, button, or the like that protrudes through the outer surface of the proximal end of the needle guard and visually shows the user whether the module is in the pre-use or ready-to-use state. It may also be a visual indicator (e.g., colors or symbols) or a tactile or audible indicator. Preferably, user noticeable indicia indicate both a pre-use priming position and a locked position of the guard after the medicated module assembly has been used to perform an injection.

Inside the bypass housing there is a cavity that contains the capsule, which comprises the single dose of medicament in the reservoir. As the needle guard is retracted during an injection, the bypass housing is moved proximally along with the capsule positioned inside the cavity, thus decreasing the cavity volume. This allows the seals of the capsule to be pierced at its top and bottom by the needle cannula such that the medicament can be expelled from the reservoir during dose delivery. When connected to the auto-injector device containing a first and second medicament and prior to piercing the seals of the reservoir, the needle cannulae are only in fluid communication with the first and second medicaments and a fluid flow path that bypasses the capsule. Preferably, a channel on the inside surface of the bypass housing is part of this fluid flow path and is used in the priming function of the drug delivery device.

As mentioned, the bypass housing preferably has one or more tracks located on the outside surface each having a set of first, second, third, and fourth paths. On the inner surface of the proximal end of the needle guard is one or more radial protrusions or drive teeth. As the guard first begins to retract, these protrusions travel in the first path causing the bypass housing to slightly rotate. As the guard continues to retract and then partially extend, the protrusions travel in the second and third paths. The protrusion moves to the fourth path and into a locking position when the guard is fully extended to its post-use position, which is preferably less extended than the starting position. The guard is rotationally constrained by the outer housing, preferably by the use of one or more spline features in the outer surface of the guard in cooperation with one or more followers or pips located at the distal end of the inner surface of the outer housing. The bypass housing is rotationally constrained when the protrusion is in the second path of the track. As the protrusion is moved axially in the proximal direction when the guard retracts, the protrusion moves from the second track to the third track causing the assembly to emit an audile sound and/or tactile feedback. This tells the user that the device will has now been activated to lock upon extension of the guard in the distal direction.

During dispense, substantially all of the medicament in the medicated module is expelled as along with the various doses of the first and second medicaments in the auto-injector device. By “substantially all” we mean that at least about 80% of the second medicament is expelled from the drug delivery device, preferably at least about 90% is expelled.

The capsule preferably contains a flow distributor to ensure that substantially all the single dose of medicament in the medicated module is forced out of the capsule by the primary and secondary medicaments during an injection. The flow distributor can be a separate stand alone insert or pin. Alternatively the flow distributor and the capsule together can be manufactured or assembled as a one-piece component where the flow distributor is integral with the capsule. Such a unitary construction can be achieved utilizing, for example, design principles such as form fit, force fit or material fit, such as welding, gluing, or the like, or any combination thereof. The one-piece component may comprise one or more medicament flow channels, preferably one flow channel. The capsule and/or flow distributor can be constructed of any material that is compatible with the primary and secondary medicaments. Preferably the capsule and/or flow distributor can be made from compatible materials of construction that include, but are not limited to, COC (an amorphous polymer based on ethylene and norbonene, also referred to as cyclic olefin copolymer, ethylene copolymer, cyclic olefin polymer, or ethylene-norbornene copolymer); LCP (a liquid crystal polymer having an aramid chemical structure that includes linearly substituted aromatic rings linked by amide groups, and further can include partially crystalline aromatic polyesters based on p-hydroxybenzoic acid and related monomers and also highly aromatic polyesters); PBT (polybutylene terephthalate thermoplastic crystalline polymer or polyester); COP (a cyclic olefin polymer based on ring-opening polymerization of norbornene or norbornene-derivatives); HDPE (high density polyethylene); and SMMA (styrene methyl methacrylate copolymer based on methyl methacrylate and styrene). A preferred material is one that is typically used to manufacture septa or pistons (bungs) found in multi-dose medicament cartridges, however, any other material that is compatible with the drug could be used, e.g., glass, plastics or specific polymers, for example, TPE (thermo plastic elastomer); LSR (liquid silicone rubber); LDPE (low density polyethylene); and/or any kind of medical grade rubber, natural or synthetic.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1a illustrates a plan view of a programmable auto-injector drug delivery device in accordance with one aspect of the present invention;

FIG. 1b illustrates a plan view of a programmable auto-injector device with an end cap removed in accordance with one aspect of the present invention;

FIG. 2 illustrates a perspective view of the device illustrated in FIGS. 1a and 1b with an end cap of the device removed;

FIG. 3 illustrates a perspective view of a cartridge holder and a back side of the device illustrated in FIG. 1b;

FIG. 4 illustrates a perspective view of a proximal end of the delivery device illustrated in FIG. 1b;

FIG. 5a illustrates a plan view of a digital display of the device after the device has been turned on but before a dose is set;

FIG. 5b illustrates a plan view of the digital display illustrated in FIG. 5a after a dose has been set;

FIG. 6 illustrates a perspective view of the device distal end showing the cartridge;

FIG. 7 illustrates a flowchart of one algorithm that can be programmed into the device illustrated in FIGS. 1a and 1b;

FIG. 8 illustrates a flowchart of another algorithm that can be programmed into the device illustrated in FIGS. 1a and 1b;

FIG. 9 illustrates a perspective view of the cartridge holder illustrated in FIG. 3 with one cartridge retainer in an open position;

FIG. 10 illustrates one type of cartridge dedication system that may be used with the cartridge holder;

FIG. 11 illustrates an interface hub that may be removably mounted on a distal end of the device illustrated in FIGS. 1a, 1b, and 2;

FIG. 12 illustrates the interface illustrated in FIG. 11 mounted on a distal end of the device illustrated in FIGS. 1a, 1b, and 2;

FIG. 13 illustrates a perspective view of the interface illustrated in FIG. 11;

FIG. 14 illustrates another perspective view of the interface illustrated in FIG. 11;

FIG. 15 illustrates a cross-sectional view of the interface illustrated in FIGS. 11 and 12;

FIG. 16 illustrates an exploded view of the interface illustrated in FIG. 11;

FIG. 17 illustrates another exploded view of the interface illustrated in FIG. 11;

FIG. 18 illustrates a cross-sectional view of the interface mounted onto an auto-injector drug delivery device, such as the device illustrated in FIGS. 1a and 1b;

FIG. 19 illustrates a block diagram functional description of a control unit for operation of the device illustrated in FIG. 11;

FIG. 20 illustrates a printed circuit board assembly of the device illustrated in FIG. 11;

FIG. 21 illustrates a schematic view of a drive mechanism for use with the device illustrated in FIGS. 1a and 1b;

FIG. 22 illustrates another schematic view of the drive mechanism illustrated in FIG. 21;

FIG. 23 illustrates a motion detection system that may be used with the drive mechanism illustrated in FIG. 21;

FIG. 24 illustrates a side view of the motion detection system illustrated in FIG. 23;

FIG. 25 illustrates a schematic view of an alternative drive mechanism for use with the device illustrated in FIGS. 1a and 1b;

FIG. 26 illustrates a schematic view of the alternative drive mechanism illustrated in FIG. 25 with certain elements removed;

FIG. 27 illustrates a schematic view of a telescope piston rod and gearing arrangement illustrated in FIG. 26;

FIG. 28 illustrates a schematic view of a telescope piston rod arrangement illustrated in FIG. 27;

FIG. 29 illustrates a schematic view of one piston rod arrangement illustrated in FIG. 27;

FIG. 30 illustrates a potential deliverable therapy of a known two input and two compound combination device;

FIGS. 31a and 31b illustrates a first arrangement of a predefined therapeutic profile that may be programmed into Applicants' programmable auto-injector drug delivery device;

FIG. 32 illustrates one arrangement of a predefined fixed ratio therapeutic profile that may be programmed into the auto-injector drug delivery device illustrated in FIGS. 1a and 1b;

FIG. 33 illustrates an alternative arrangement of a predefined fixed ratio therapeutic profile that may be programmed into an auto-injector drug delivery device comprising three medicaments;

FIG. 34 illustrates an alternative arrangement of a predefined fixed ratio therapeutic profile that may be programmed into an auto-injector drug delivery device comprising four medicaments;

FIG. 35 illustrates another alternative arrangement of a predefined fixed ratio therapeutic profile having discrete dose steps and that may be programmed into the auto-injector drug delivery device illustrated in FIGS. 1a and 1b;

FIG. 36 illustrates an arrangement of a predefined non-linear fixed ratio therapeutic profile having a decreasing rate of change and that may be programmed into the auto-injector drug delivery device illustrated in FIGS. 1a and 1b;

FIG. 37 illustrates an alternative arrangement of a predefined non-linear fixed ratio therapeutic profile having a decreasing rate of change and that may be programmed into the auto-injector drug delivery device illustrated in FIGS. 1a and 1b;

FIG. 38 illustrates an arrangement of a predefined non-linear fixed ratio therapeutic profile having an increasing rate of change and that may be programmed into the auto-injector drug delivery device illustrated in FIGS. 1a and 1b;

FIG. 39 illustrates an alternative arrangement of a predefined non-linear fixed ratio therapeutic profile having an increasing rate of change and that may be programmed into the auto-injector drug delivery device illustrated in FIGS. 1a and 1b;

FIG. 40 illustrates an arrangement of a predefined fixed ratio-fixed dose therapeutic profile having a low dose threshold and that may be programmed into the auto-injector drug delivery device illustrated in FIGS. 1a and 1b;

FIG. 41 illustrates an alternative arrangement of a predefined fixed ratio-fixed dose therapeutic profile having a high dose threshold and that may be programmed into the auto-injector drug delivery device illustrated in FIGS. 1a and 1b;

FIG. 42 illustrates an alternative arrangement of a predefined fixed ratio-fixed dose therapeutic profile having a low dose threshold and that may be programmed into an auto-injector drug delivery device for use with at least three medicaments;

FIG. 43 illustrates an arrangement of a predefined fixed dose-variable dose therapeutic profile that may be programmed into the auto-injector drug delivery device illustrated in FIGS. 1a and 1b;

FIG. 44 illustrates an alternative arrangement of a predefined fixed dose-variable dose therapeutic profile that may be programmed into an auto-injector drug delivery device and for use with at least three medicaments;

FIG. 45 illustrates an arrangement of a predefined delayed fixed dose-variable dose therapeutic profile having a low threshold and that may be programmed into the auto-injector drug delivery device illustrated in FIGS. 1a and 1b;

FIG. 46 illustrates an arrangement of a predefined delayed fixed dose-variable dose therapeutic profile having a high threshold and that may be programmed into the auto-injector drug delivery device illustrated in FIGS. 1a and 1b;

FIG. 47 illustrates an alternative arrangement of a predefined delayed fixed dose-variable dose therapeutic profile having a low dose threshold and that may be programmed into the auto-injector drug delivery device illustrated in FIGS. 1a and 1b;

FIG. 48 illustrates an arrangement of a predefined delayed fixed dose-variable dose therapeutic profile having offset dose thresholds and that may be programmed into the auto-injector drug delivery device illustrated in FIGS. 1a and 1b;

FIG. 49 illustrates an arrangement of a predefined multi-level fixed dose-variable dose therapeutic profile having a slow ramp up and that may be programmed into the auto-injector drug delivery device illustrated in FIGS. 1a and 1b;

FIG. 50 illustrates an arrangement of a predefined multi-level fixed dose-variable dose therapeutic profile having a fast ramp up and that may be programmed into the auto-injector drug delivery device illustrated in FIGS. 1a and 1b.

FIG. 51 illustrates an example of the medicated module of the present invention;

FIG. 52 illustrates an exploded distal perspective view of all the components (except the medicated capsule) of the medicated module illustrated in FIG. 51;

FIG. 53 illustrates an exploded proximal perspective view of all the components (except the medicated capsule) of the medicated module illustrated in FIG. 51;

FIG. 54 is a perspective view of the capsule containing the reservoir of the medicated module illustrated in FIG. 51;

FIG. 55 illustrates a proximal perspective view of the outer housing of the medicated module illustrated in FIG. 51;

FIG. 56 is a sectioned view of the medicated module illustrated in FIG. 51 orientated in the bypass configuration;

FIG. 57 is a close-up perspective view of the bypass housing of the medicated module illustrated in FIG. 51 to illustrate the positions of the drive tooth during use;

FIG. 58 illustrates an example of a reservoir and flow distributor that may be used with the medicated module illustrated in FIG. 51;

FIG. 59 illustrates a perspective view of the medicated module illustrated in FIG. 51;

FIG. 60 illustrates an exemplary drug delivery system including the auto-injector drug delivery device illustrated in FIGS. 1a and 1b and the medicated module illustrated in FIG. 51.

### DETAILED DESCRIPTION

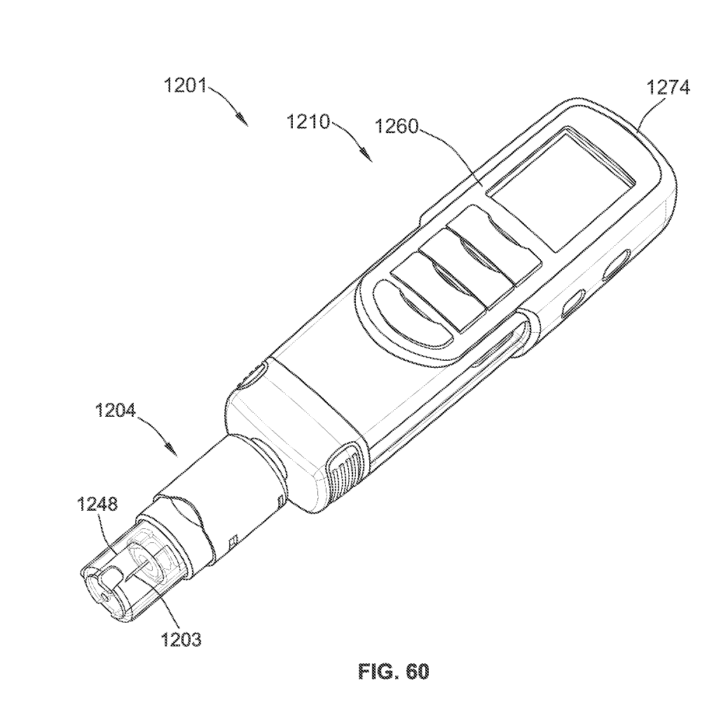

The disclosed drug delivery system and corresponding method allow for the delivery of a combination dose comprising three or more medicaments and/or fluids. As disclosed herein, and with reference to FIG. 60, the system 1 includes two major components: an auto-injector device 10 that contains at least two medicaments (e.g., a first and a second medicament) and a medicated module 1204 that contains at least one medicament (e.g., a third medicament). The medicated module 1204 interfaces with the auto-injector device 10 such that all three medicaments can be delivered via a single dispense interface 1203 of the medicated module 1204.

Upon attaching the medicated module 1204 to the auto-injector 10, a fixed dose of the third medicament is set based on the amount of the third medicament within the reservoir of the medicated module 1204. The user then sets a user-settable dose of the first medicament using the dose setter of the auto-injector 10 (e.g., buttons on the control panel 60), which causes a dose of the second medicament to be set according to a predefined therapeutic dose profile. After the combination dose is set, the user presses the distal end of the needle guard 1248 of the medicated module 1204 against the skin of the user such that the needle guard 1248 retracts and the dispense interface 1203 penetrates the skin of the user. A pre-defined amount of needle guard retraction places all three medicaments in fluid communication with the dispense interface 1203. The user then activates the system 1 (e.g., actuates a button 74 on the auto-injector 10), which causes the first and second medicaments to flow through the medicated module 1204 thus forcing the third medicament out of the medicated module 1204 and thereby delivering the combination dose via the dispense interface 1203. In one example, the auto-injector device 10 contains a first cartridge containing a long acting insulin and a second cartridge containing a short acting insulin, and the reservoir of the medicated module 1204 contains a GLP-1.

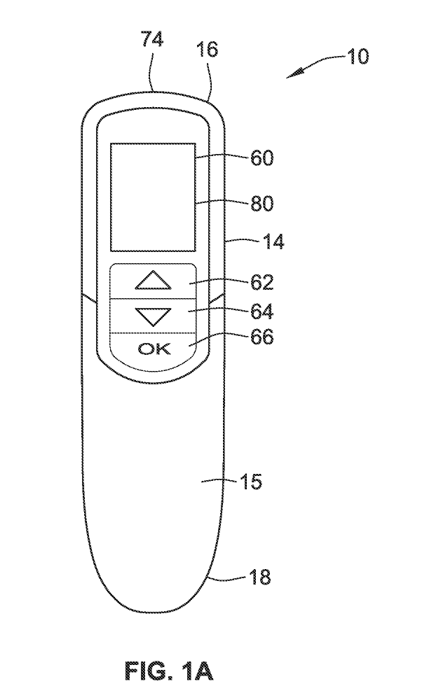

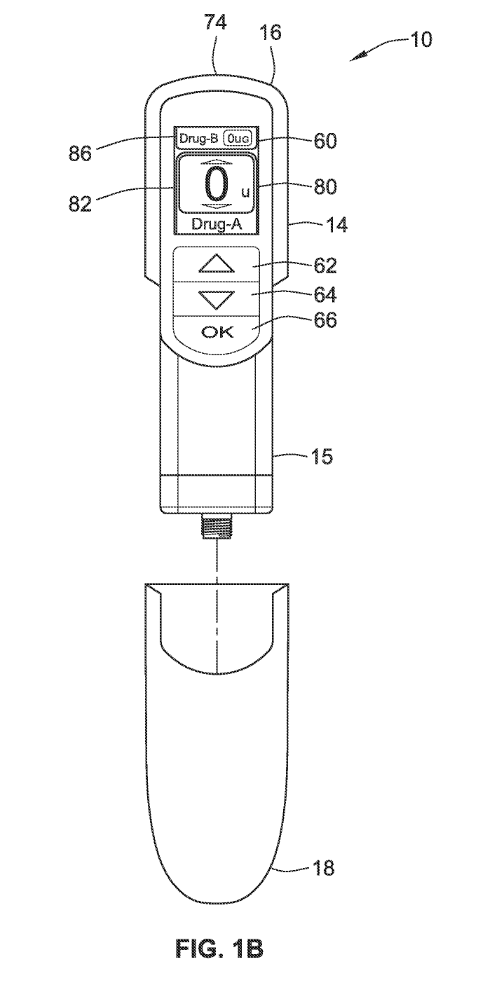

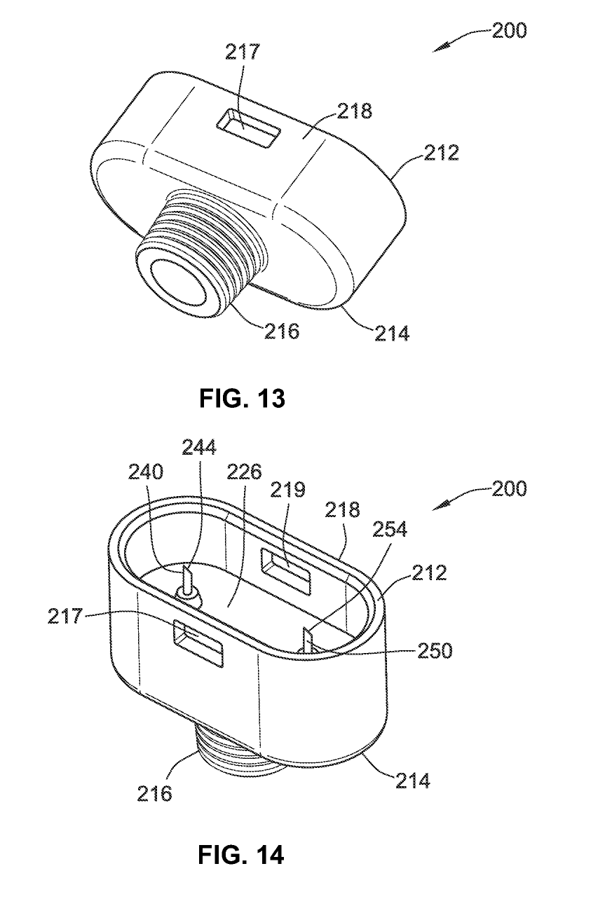

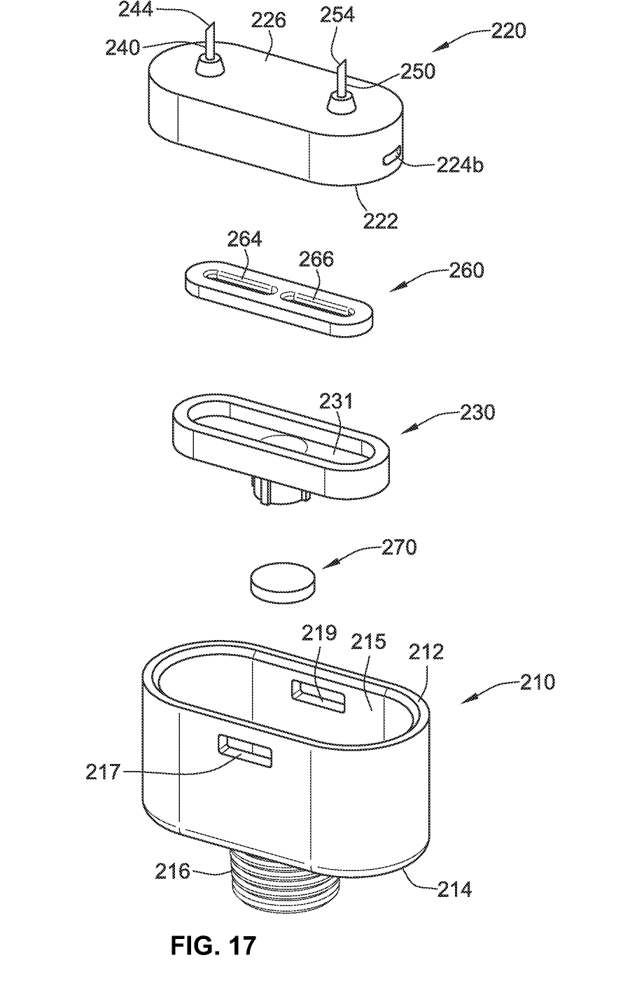

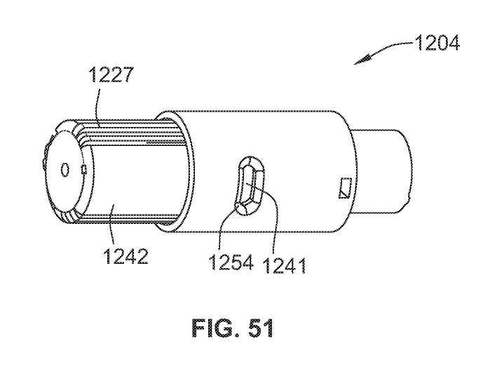

For sake of clarity, the details of the auto-injector device and the medicated module will be described separately with the auto-injector being described first with reference to FIGS. 1-50 and the medicated module being described thereafter with reference to FIGS. 51-59.

### A. Auto-Injector Device

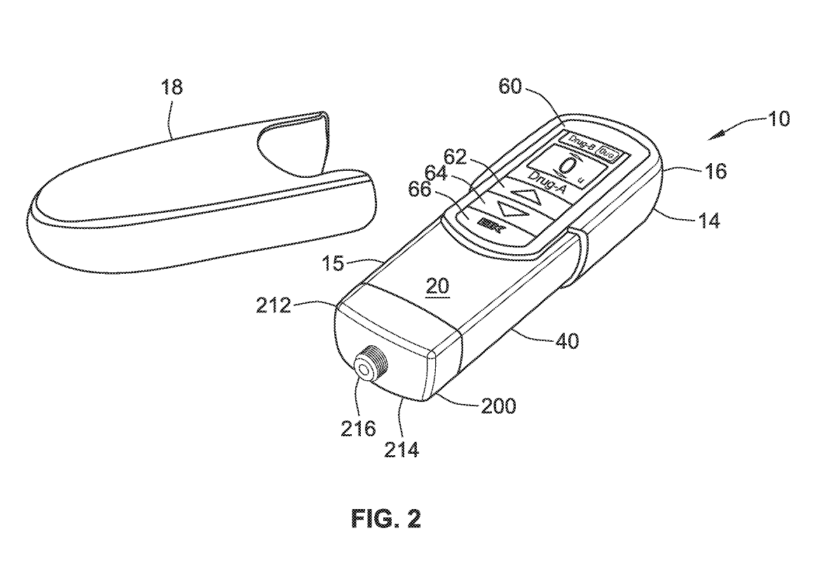

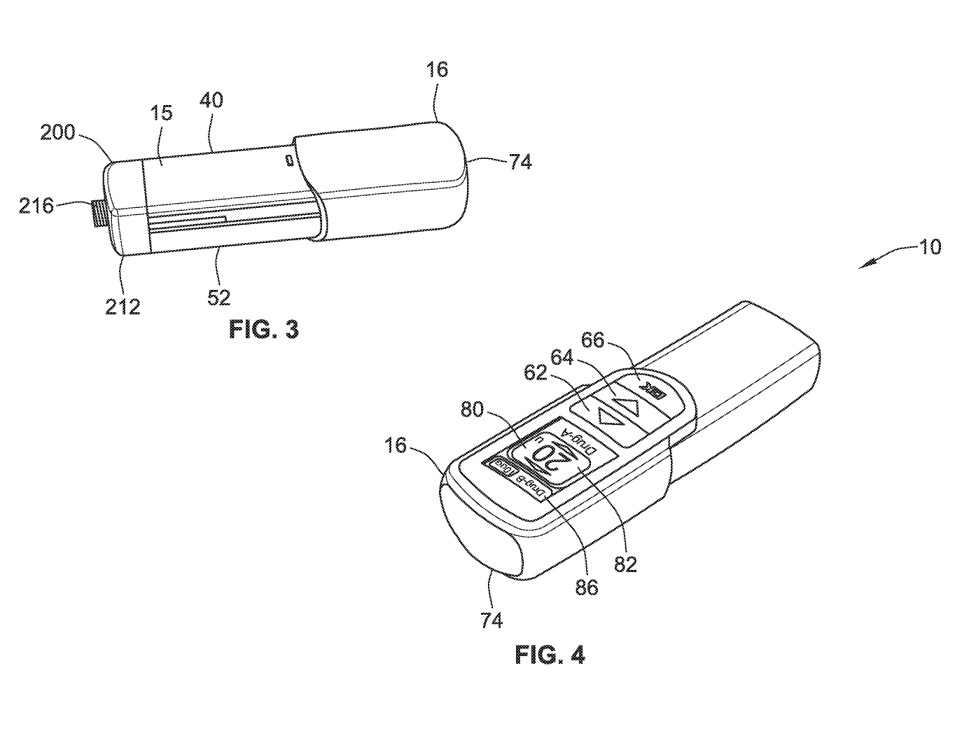

FIGS. 1a and 1b illustrate plan views of a programmable auto-injector drug delivery device 10 in accordance with one aspect of the present invention. FIG. 1a illustrates the device 10 when an end cap 18 is on the device 10. In FIG. 1b, the device 10 is illustrated in a ready mode in that the end cap 18 is off and the device 10 has been turned on so that the digital display 80 is illuminated. When the device 10 is activated with the cap 18 on, only cartridge contents, battery status and last dose information will be available for display. However, when the cover 18 is removed and the device 10 is activated, the dose setting screen will be available. FIG. 2 illustrates a perspective view of the delivery device 10 shown in FIGS. 1a and 1b with the end cap 18 of the device 10 removed. In FIG. 2, the device 10 is turned on so that the digital display 80 is illuminated. FIG. 3 illustrates a perspective view of the cartridge holder 40 and the back side of the delivery device 10 illustrated in FIGS. 1a and 1b. FIG. 4 illustrates a perspective view of a proximal end of the delivery device 10.

Referring now to FIGS. 1 through 4, there can be seen a micro-processor controlled electro-mechanical auto-injector drug delivery device 10 in accordance with the present invention. Preferably, this drug delivery device 10 is generally rectangular in shape comprising generally rounded ends so as to easily fit in a user's shirt pocket and is also compact enough to fit in a hand bag.

As will be described in greater detail below, the drug delivery device 10 contains a micro-processor control unit that operates an electro-mechanical drive that is used to deliver at least two drugs (e.g., a first or primary medicament and a second or secondary medicament) during a single dosing operation. This enables the drug delivery device 10 to provide, for example, a primary medicament such as a long acting insulin along with a secondary medicament such as a GLP1 as a combination therapy. Such combination therapy may be defined by one of a plurality of therapeutic profiles stored in a memory device that is coupled to the micro-processor contained within the device 10.

The drug delivery device illustrated in FIGS. 1 through 4 comprises a main body 14 that extends from a proximal end 16 to a distal end 15. At the distal end 15, a removable end cap or cover 18 is provided. This end cap 18 and the distal end 15 of the main body 14 work together to provide a snap fit or form fit connection so that once the cover 18 is slid onto the distal end 15 of the main body 14, this frictional fit between the cap and the main body outer surface 20 prevents the cover from inadvertently falling off the main body. Other types of connection mechanisms may also be used such as frictional fits or snap fits provided by way of a clip feature.

As will be described in greater detail below, the main body 14 contains a micro-processor control unit, an electro-mechanical drive train, and at least two medicament reservoirs. When the end cap or cover 18 is removed from the device 10 (as illustrated in FIGS. 1b, 2, 3, and 4), interface 200 (see FIG. 3), which is mounted to the distal end 15 of the main body 14, is accessible. A medicated module (which will be described in detail below) containing a third medicament can then be attached to the interface 200. Once the medicated module is attached to the device 10 via the interface 200, the system is capable of administering a variable dose of a first medicament (primary drug compound), a computed dose of a second medicament (secondary drug compound), and a fixed dose of a third medicament through a single dispense interface of the medicated module.

A control panel region 60 is provided near the proximal end 16 of the main body 14. Preferably, this control panel region 60 comprises a digital display 80 along with a plurality of human interface elements that can be manipulated by a user to set and inject a combination dose. In this arrangement, the control panel region comprises a first dose setting button 62, a second dose setting button 64, and a third button 66 designated with the symbol “OK.” As illustrated, the first dose setting button 62 resides above the second dose button 64, which is positioned above the OK button 66. Alternative button arrangements may also be used. As just one example, the first button 62 and a second button 64 may, as a pair, be rotated through 90 degrees and sit underneath the screen, with each button being adjacent to a screen area. In such an arrangement, the first and second buttons could be used as soft keys to interact with icons on the user digital display 80. In addition, along the most proximal end of the main body, an injection button 74 is also provided (see e.g., FIG. 4).

Utilizing micro-processor controlled human interface elements such as an operator panel (e.g., hard keys, buttons or soft keys with the key legend appearing on the display screen), setting the dose of the primary medicament allows the control unit to compute or determine the fixed dose of the second medicament. In one preferred arrangement, a computerized electronic control unit computes the dose of the second medicament. The computerized electronic control unit computes the dose of the second medicament based at least in part on a therapeutic dose profile that is stored in a memory device coupled to the micro-processor. Such a therapeutic profile may or may not be user or caregiver selectable. As will be explained in greater detail below, a plurality of different such dose profiles may be stored on a memory storage device in the drug delivery device 10. In one arrangement, the preferred memory storage device comprises Flash memory of the micro-processor. An optional storage device could comprise an EEPROM that is coupled via a serial communication bus to the micro-processor of the control unit.

FIG. 2 illustrates a perspective view of the drug delivery device 10 of FIGS. 1a and 1b with the cover 18 removed so as to illustrate the main body 14 and a cartridge holder 40. By removing the cover 18 from the device, a user is provided access to the cartridge holder 40 and also to the interface 200. In one preferred arrangement, this cartridge holder 40 can be removably attached to the main body 14. In this arrangement, and as illustrated in FIG. 6, the cartridge holder 40 contains two cartridge retainers 50 and 52. Each retainer is configured so as to contain one medicament reservoir, such as a glass cartridge. Preferably, each cartridge contains a different medicament. In alternative drug delivery device arrangements, more than two cartridge retainers may be contained within the cartridge housing.

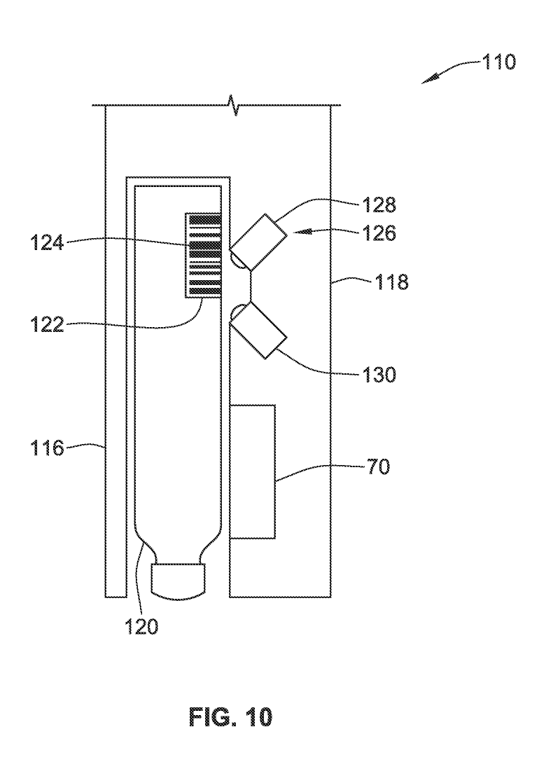

In one arrangement, each cartridge retainer 50, 52 may be provided with a cartridge detecting system, such as the cartridge detecting system illustrated and described with respect to FIG. 10. Such a cartridge detecting system may comprise a mechanical or electrical switch that can be used to determine if a cartridge has been correctly inserted into the retainers 50, 52. Ideally, such a detection system can determine if the correct size cartridge has been properly inserted into the retainer.

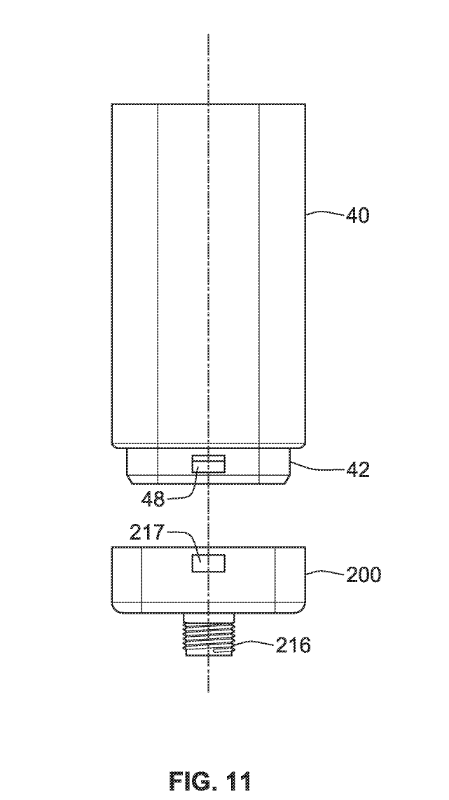

In addition, at the distal end of the cartridge holder 40, the drug delivery device illustrated in FIG. 2 includes an interface 200. As will be described in relation to FIG. 11, this interface 200 includes a main outer body 212 that is removably attached to a distal end 42 of the cartridge housing 40. As can be seen in FIGS. 2 and 3, a distal end 214 of the interface 200 comprises a needle hub 216. This needle hub 216 is configured so as to allow a medicated module to be removably mounted to the drug delivery device 10.

As noted above, at a first or a proximal end 16 of the main housing 14, there is provided a control panel region 60. This control panel region 60 comprises a digital display, preferably an Organic Light Emitting Diode (OLED) display 80 along with a plurality of user interface keys such as push buttons. Alternatively, this region could comprise a touch screen and icons on the display. A further option would be a display screen with a joystick, a control wheel and/or possibly push buttons. In addition, the control panel region may also comprise a swipe section so as to either increase or decrease the dose size or provide other means by which a user could operate the device 10. Preferably, the human interface controls may be configured to provide tactile, audible and/or visual feedback.

The digital display 80 may be part of a user interface that allows the user to interact with the device 10. As explained in greater detail below, this display provides a visual indication of device operation such as dose setting, dose administration, injection history, device errors, etc. The digital display 80 can also display various drug delivery device parameters. For example, the display can be programmed to display an identified medicament contained in either medicament containers and also provide a visual confirmation that the correct cartridge and therefore medicament is being used. In addition, the display can also provide dose history information such as the time since the last dose has been administered, battery level, dose size set, device status, dose dispense status, dose history information, warnings, and errors.

Further, the display 80 may also provide the time and date and be used to set a current time and date. The display may also be used to provide the user with training information as to how the device should be used and operated. Alternatively or additionally, the display may be used to educate the user on diabetes or other therapy information via instructional videos. The display may also be used to communicate with, or receive feedback from a health care professional via the wireless or wired communication link such as USB to a PC and then potentially via the internet, or via a mobile phone coupled to the device using a wired or wireless link such as a Bluetooth™ link, a WLAN link, and/or the like. The display may also be used to configure a device communication link: that is, used for device set up and enter passwords for a data link, such as a Bluetooth data link. In addition, the display may be used to provide drug delivery device priming information or possibly an indication of the orientation and/or relative position of the device. For example, a micro-electro-mechanical accelerometer could be provided within the device so that the device will have the intelligence to know if the user is using the device to perform a safety or priming shot (i.e., having the distal end of the device pointing upwards) or using the device to perform a dose administration step (i.e., having the distal end of the device pointing downwards).

The display may also potentially be used as a diary or life style calendar and perhaps communicate with a patient's BGM and perhaps store and display blood glucose data. The display could also indicate a dwell period, possibly proportional to a dose size, following the delivery of a dose. The display could indicate if the device is armed i.e., ready to deliver a dose and also be used to provide an indication if the dose is outside of expected limits.

In addition, by manipulating certain other buttons, the display can be used to display information stored in the control unit. For example, such stored information could include user or patient information. Such user or patient information could include their name, their address, their health number, contact details, their prescribed medication or dosage regime.

In addition, there is also the opportunity to include calendar information, which could include blood glucose readings, the size of last dose taken, exercise taken, state of health, the time these events occurred including meal times, etc. Certain key events can also be stored and viewed. For example, such key events could include device failures that could potentially result in an over or under dose, cartridge changes, priming shots, reading the dose history, removing the cap, removing the dose dispenser, removing the interface, removing the medicated module, time since manufacture, time since first use along with other similar types of information and data.

The digital display could also allow the user access to a time reference maintained by the device. Such a time reference could keep track of the current time and date. This clock may be set by the user via the interface or alternatively, via a data link (e.g., USB or IRDA) provided on the device. In addition, the time reference may be provided with a permanently connected battery backup so as to maintain the passage of time if and when the main battery has been removed or is flat. This time reference may be used to determine when the last dose was taken, which can then be displayed on the display. This time reference may also be used to store certain key events. Such events could include the time and date of the following: the last dose; whether any drug delivery device errors occurred; cartridge changes; any parameter changes, any changes in therapeutic profiles; interface changes; medicated module changes, and time since manufacture.

As previously mentioned, FIG. 1b illustrates one arrangement of the drug delivery device 10 after the user has turned the device on. One way in which a user may turn the device on is for the user to press the “OK” button 66 provided on the control panel region 60. Alternatively, the device 10 can be programmed to be turned on by removing the end cap 18. The OK button 66 may then be used when the device 10 has gone into a sleep mode after a certain period of inactivity. The sleep mode may be indicated by a possibly blank display screen. Preferably, when the cap 18 is placed back upon the device, it may be possible to review via the display 80 certain dose or dosing history data by pressing one of the human interface elements, such as the OK button 66.

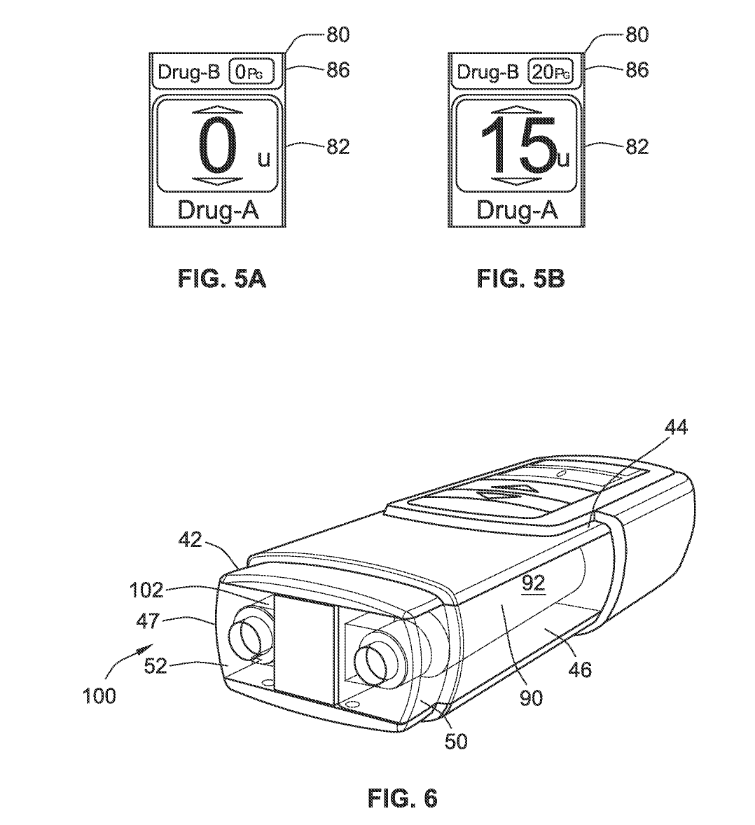

Once the device is turned on, the digital display 80 illuminates and provides the user certain device information, preferably information relating to the medicaments contained within the cartridge holder 40. For example, as illustrated in FIGS. 1 and 5, the user is provided with certain information relating to both the primary medicament (Drug A) and the secondary medicament (Drug B). Preferably, the display comprises at least two display regions 82, 86 containing medicament information. The first display region 82 provides the user information relating to the primary medicament: the type of medicament—“Drug A” and the amount of Drug A that has been selected by the user—“0 Units.” In addition, the second display region 86 provides the user with information relating to the secondary medicament: the type of medicament—“Drug B” and the amount of Drug B that has been calculated by the device based on the amount of Drug A selected by the user and on the particular therapeutic profile—“0μGrams.” As those of ordinary skill in the art will recognize, if in an alternative arrangement, the drug delivery device 10 contained three medicaments and was used to administer a combination therapy of these three medicaments (not including the medicament in the medicated module), the digital display 80 would be modified so as to comprise at least three display regions containing information for at least these three medicaments.

Where the size of the second dose is determined from the size of the first it may not be necessary to indicate the size of the second dose and hence an alternative embodiment of the display graphics may be used, for example an “O.k.” indication, such as a green dot, a green check mark, or the letters “O.k.”.

Aside from the digital display 80, the control panel region 60 further comprises various user interface keys. For example, as illustrated in FIGS. 1a, 1b, 2 and 4, the control panel region 60 of the drug delivery device 10 further provides the following user interface keys:
a. a first dose setting button 62,
b. a second dose setting button 64, and
c. an OK or Enter button 66.

The first and second dose buttons 62, 64 may be manipulated so as to allow a user of the device 10 to either increase or decrease a selected dose of the primary medicament “Drug A” to be delivered. For example, to set or increase a primary medicament dose amount, a user could toggle the first dose setting button 62. The first display region 82 would provide a visual indication to the user of the amount he or she is setting.

In the event that a user wants to decrease a previously set dose, the second dose setting button 64 may be toggled or pushed so as to decrease the set dose. Once the user has selected the amount of the primary medicament, the user may then push the “OK” button 66. Pushing the OK button 66 may instruct the device 10 to compute the corresponding dose of the secondary medicament “Drug B”. Alternatively, the dose of the secondary medicament may be determined when the dose of the first medicament is set or changed.

In an alternative display arrangement, the display 80 can display the calculated amount of the secondary medicament Drug B for every incremental change of Drug A.

Thereafter, the OK button 66 could then be used. For example, pressing and holding this OK button 66 for a certain period of (e.g., 2 seconds) could be used by the user to confirm the set and calculated dose and thereby arming the device 10 ready for delivery.

The combined dose, including the fixed dose of medicament in the medicated module, could then be dispensed through a dispense interface of the medicated module by pressing the injection button 74. In one preferred arrangement, the device armed condition may be available for a limited period, for example, 20 seconds or so. In an alternative arrangement, the arm feature may not be included.

FIG. 5a illustrates the display 80 of device 10 illustrated in FIG. 1b after the device has been turned on but before a user sets a first dose of the primary medicament Drug A. FIG. 5b illustrates this display 80 after a user has set a first dose of the primary medicament Drug A and after the device has computed the corresponding amount of the secondary medicament Drug B. As illustrated in FIG. 5b, the user has set a 15 Unit dose of the primary medicament Drug A and this is confirmed by what is displayed in the first display region 82. After the device 10 computes the secondary dose of the second medicament Drug B, this is also indicated by what is displayed in the second region 86. For example, in this situation, the device 10 calculated a dose of 20 μGrams for Drug B based in part on a 15 Unit dose of the primary medicament Drug A and based in part on one of the algorithms stored within the device.

This combined dose, 15 Units of the primary medicament Drug A and 20 μGrams of the secondary medicament Drug B, can then be injected along with the fixed dose of medicament in the medicated module. As may be seen from FIG. 4, at a proximal end 16 of the main body 14 of the device 10, an injection button 74 is provided for injecting this combined dose. Alternatively, this dose inject button 74 could be provided elsewhere on the main housing 14 such as on the control panel region 60.

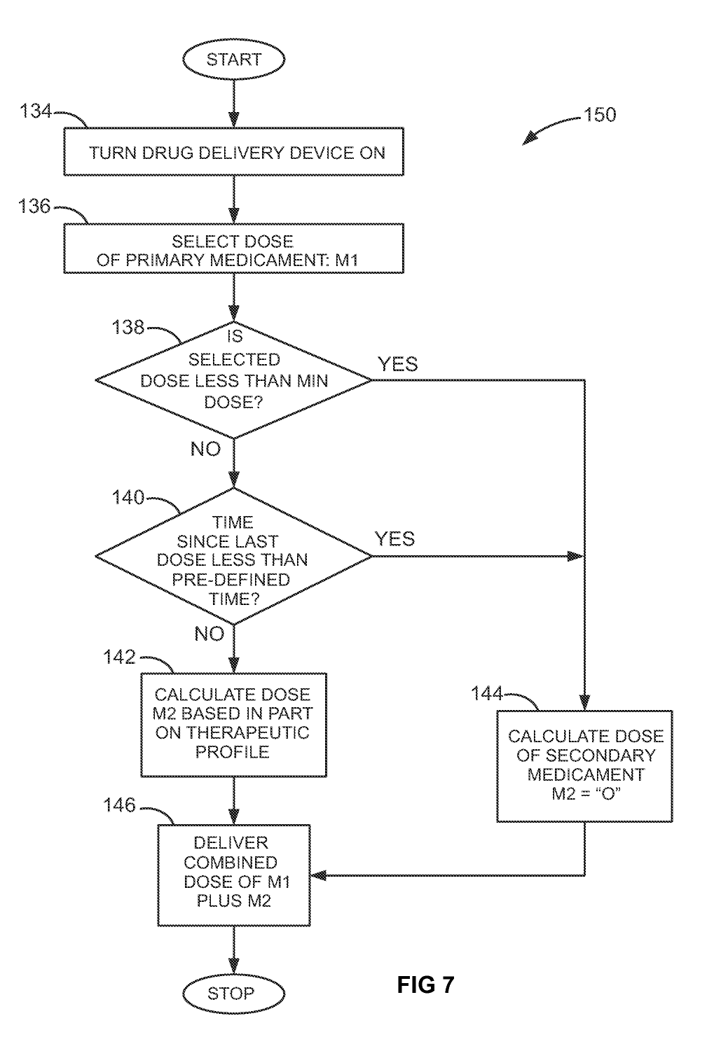

Other information that may be taken into account when calculating the amount of the second medicament may be the time interval since the previous dose of either the first or the second medicament. For example, the following description provides an example algorithm and process that may be used in the calculation of the size of the dose to be dispensed from the second medicament. This algorithm maybe illustrated in a flowchart 150 provided as FIG. 7.

As may be seen from the flowchart 150 provided in FIG. 7, first, a user begins the dose selection process by turning the device on at step 134. Then, at step 136, the user selects the size of the dose to be delivered from the first medicament M1 in the first cartridge and then presses the OK button to confirm. At step 138, the microcontroller determines if the selected dose size of the first medicament M1 is less than a minimum dose threshold for the first medicament (e.g., 5 units). If it is determined that the selected dose size is indeed less than the minimum dose threshold, the process proceeds to step 144 where the calculated dose of the second medicament M2 is then computed as a zero dose. Then, the process moves to step 146 where the dose (comprising only a selected dose of the primary medicament) is administered.

If the selected dose size is determined to be greater than or equal to this minimum dose threshold, the process 150 proceeds to step 140. At step 140, the microcontroller determines if the time interval since the previous injection is less than, or equal to the predefined threshold (e.g., 18 hours). If the answer to this inquiry is yes, the process 150 proceeds to step 144 where the size of the dose from the second medicament M2 would be calculated as equal to a zero (“0”) dose. Then, the process moves to step 146 where the dose (comprising only a selected dose of the primary medicament) is administered.

Alternatively, if the answer to both inquiries at steps 138 and 140 are no, then process 150 would proceed to the step 142. At step 142, the microcontroller would compute the dose of the secondary medicament M2 based at least in part on a stored therapeutic profile. If an additional medicament and/or fluid is provided in the auto-injector device, the microcontroller would compute a dose of the additional medicament based at least in part on a stored therapeutic profile as well. This later profile may or may not be the same profile that is used to calculate the dose of the secondary medicament.

Therefore, if a user selects a dose size of the primary medicament M1 at step 136 that is equal to, or greater than, a certain minimum dose threshold for the first medicament (e.g., 5 units), and the time interval since the previous injections is greater than the predefined threshold (e.g., 18 hours) then the predefined dose of the secondary medicament from the second cartridge (e.g., 0.5 units) will be delivered when the injection is administered at step 146.

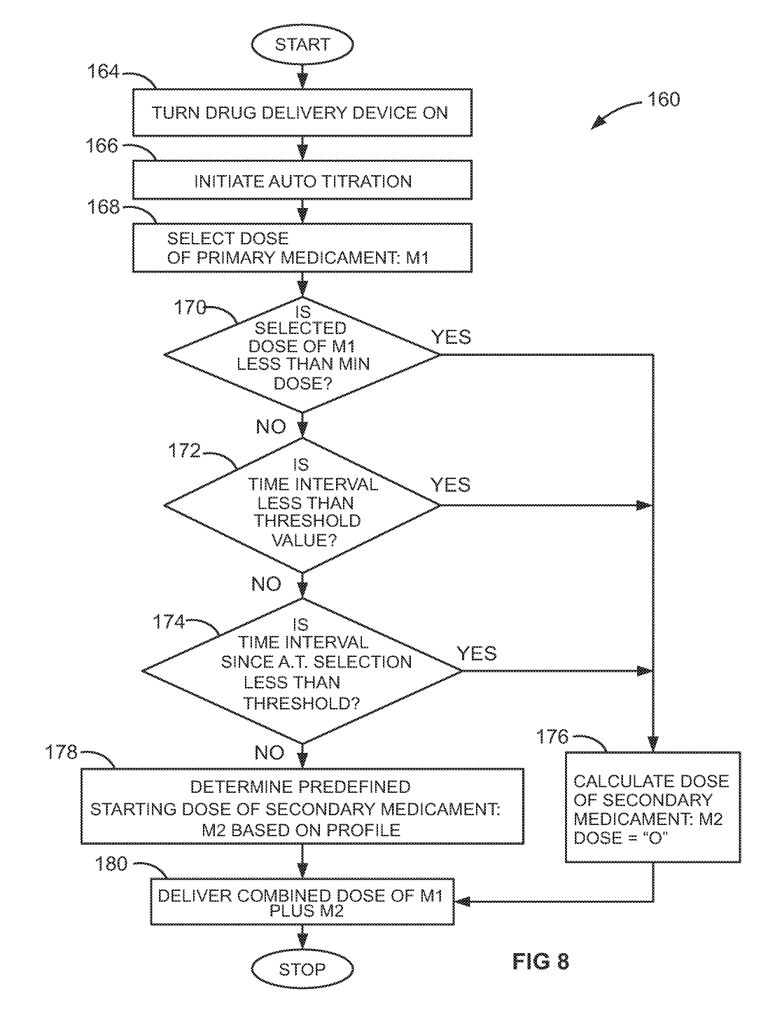

Applicants' drug delivery device 10 may also be programmed with an auto titration algorithm. As just one example, such an algorithm may be used where the dose of the second medicament needs to be increased over a period of time to allow a patient to get used to the second medicament, such as is the case for a GLP1 or GLP1 analogs. An exemplary auto titration algorithm is presented in a flowchart 160 illustrated in FIG. 8.

In one arrangement, after the device is turned on at step 164, a user initiates an auto titration mode of operation by manipulating one of the keys provided on the control panel. This is represented at step 166. Alternatively, this auto titration mode of operation could be automatically activated. For example, the auto titration mode of operation could be automatically activated when the drug delivery device 10 is first used, for example, when a battery is first connected to the device, when the battery is first charged, or when a profile is loaded into the device and selected by a user. After step 166, a prompt on the digital display 80 may ask a user for a password and then to confirm that the auto titration algorithm is indeed desired by the patient. In an alternative embodiment, a prompt on the digital display 80 may ask the user for a confirmation only. Aside from using a stored algorithm for operating the device in an auto titration mode, this auto titration mode might be achieved via providing a user with cartridges containing the same medicament but with different strengths or concentrations. One disadvantage of such a scenario is that the provider of such cartridges would have to produce cartridges in at least two different strength concentrations of drugs rather than through smaller doses from a standard strength cartridge. If different strength cartridges are used, then the device may be programmed not to provide the auto-titration functionality. If this functionality is optional and patient determined, then such a function could be accessed through the digital display 80 via a ‘menu’ button (or other similar user interface element).

At step 168, a user selects a dose of the primary medicament M1. Then, at step 170, the microcontroller determines if the selected dose size is less than a minimum dose threshold for the first medicament (e.g., 5 units). If the microcontroller determines that the selected dose size is less than a minimum dose threshold for the first medicament, the process 160 proceeds to step 176. At step 176, the microcontroller determines that the calculated dose of the secondary medicament M2 should be a zero (“0”) dose.

If at step 170 the microcontroller determines that the selected dose size of M1 is not less than a minimum dose threshold for the first medicament, the process 160 proceeds to step 172. At step 172, the microcontroller computes a time interval since the previous dose administration and determines if this computed time interval is less than, or equal to a predefined threshold (e.g., 18 hours). If at step 172 the microcontroller determines that this computed time interval is less than, or equal to a predefined threshold, the process 160 proceeds on to step 176. At step 176, the microcontroller determines that the calculated dose of the secondary medicament M2 should be a zero (“0”) dose.

Alternatively, if at step 172, the microcontroller determines that this computed time interval since the previous injection is not less than, or equal to a predefined threshold, the process proceeds to step 174.

If the microcontroller determines that the selected dose size is equal to, or greater than, the minimum dose threshold for the first medicament (e.g., 5 units) at step 170 and determines that the time interval since the previous injection is greater than the predefined threshold (e.g., 18 hours) at step 172, the process proceeds to step 174. At step 174, the microcontroller determines whether the time interval since the auto-titration feature was activated is less than a predefined threshold (e.g., 1 week). If at step 174 the microcontroller determines that the time interval since the auto-titration feature was activated is greater than this predefined threshold, the process 160 moves to step 176 where a zero “0” dose of M2 is determined.

Alternatively, if the microcontroller determines that the time interval since the auto-titration feature was activated is less than the predefined threshold at step 174, the process moves to step 178. At step 178, the microcontroller determines a predefined starting dose of the secondary medicament based in part on a therapeutic profile. Then, at step 180, the predefined starting dose from the second cartridge (e.g., 0.25 micro Grams) M2 along with the previously selected dose of the primary medicament M1 from step 168 will be delivered during an injection step.

Therefore, in accordance with the auto titration flowchart 160, if the selected dose size is equal to, or greater than, the minimum dose threshold for the first medicament (e.g., 5 units) and the time interval since the previous injections is greater than the predefined threshold (e.g., 18 hours) and the time interval since the auto-titration feature was activated is greater than a predefined threshold (e.g., 1 week) then the predefined maintenance dose from the second cartridge (e.g., 0.5 units) will be delivered when the injection is taken at step 180. If the calculated responses to the steps 170 and 172 are yes or if the response to step 174 is no, then the dose that is administered would comprise only the selected dose of the primary medicament from step 168.

Aside from the user interface keys, the drug delivery device may also comprise a sounder or a sound control. For example, the device may have a sounder that generates a range of tones. Such tones could be provided so as to indicate when a button is pressed, when certain key events occur (e.g., after a dose is set, after the completion of a dose delivery, etc.), warnings that the device is not working correctly or if an incorrect cartridge has been inserted, if the device experiences certain operational errors, or if an alarm condition is triggered. The volume of the sounder may be set or configured by using a menu system controlled by the human interface elements or alternatively through a dedicated volume control button.

As noted above, the main housing portion 14 is preferably coupled to a proximal end of the cartridge holder 40. As shown in FIG. 6, cartridge holder 40 comprises two separate cartridge retainers 50, 52 that are configured to hold two reservoirs of medicament 90, 100. Depending on the reservoirs, these two retainers may or may not be similarly sized. FIG. 3 illustrates a back side of the drug delivery 10 illustrated in FIGS. 1a and 1b and illustrates one of the cartridge retainers 52. FIG. 6 illustrates a distal end of the cartridge holder of the drug delivery device illustrated in FIGS. 1a and 1b and illustrates both the first and the second cartridge retainers 50, 52. The first cartridge retainer 50 is configured for receiving a first cartridge 90 containing a primary medicament 92 and the second cartridge retainer 52 is configured for receiving a second cartridge 100 containing a secondary medicament 102. The first and second cartridges 90, 100 may or may not be of similar size and/or dimensions.

As illustrated in FIG. 6, the cartridge housing 40 comprises a first window 46 residing along a first side portion of the cartridge housing. Similarly, the cartridge housing 40 comprises a second window 47 residing along a second side portion of the cartridge housing 40. The two cartridge retainers 50, 52 are positioned essentially side-by-side. Once the cap 18 is removed from the drug delivery device 10, the windows 46, 47 enable a user to view the medicaments contained within the cartridges and monitor the amount of medicament remaining in each reservoir. For example, as may be seen from FIG. 6, the first window 46 allows the user to monitor the primary medicament 92 contained within the first cartridge 90 while the second window 47 allows the user to monitor the second medicament 102 contained within the second cartridge 100. The visible cartridge contents could be confirmed by what is displayed on the digital display 80.

In this illustrated arrangement, the first cartridge 90 contains a primary medicament 92 and the second cartridge 100 may contain a secondary medicament 102. In one arrangement, both the first and the second cartridges contain multiple doses of each medicament 92, 102, respectively. Each cartridge is self-contained and provided as a sealed and sterile cartridge. These cartridges can be of different volumes and replaceable when empty or they can be fixed (non-removable) in the cartridge holder 40. They can also have a pierceable seal or septa at a distal end of the cartridge and configured to accept needle cannula (e.g., needle cannula of interface 200).

Various cartridge holder arrangements may be used with the drug delivery device illustrated in FIGS. 1-6. As just one example, the cartridge holder 40 may comprise separately shaped cartridge retainers 50, 52. As just one example, the first cartridge retainer 50 may be shaped to receive a cartridge having a first volume while the second cartridge retainer 52 may be shaped to receive a cartridge having a second volume.

The primary medicament 92 contained in the first cartridge 90 may comprise a long acting insulin whereas the second medicament 102 contained within the secondary cartridge 100 may comprise a GLP1 or like analog.

As such, in one arrangement, the volume of the first cartridge 90 may be a standard 300 Unit cartridge and therefore the first cartridge retainer 50 must be geometrically configured for such a volume. In contrast, the volume of the second cartridge 100 may be a smaller volume (e.g., in the order of 20 Units) and therefore must be geometrically configured to receive such a smaller volume cartridge. As those of ordinary skill in the art with recognize, other cartridge and cartridge retainer arrangements and geometries are possible as well.

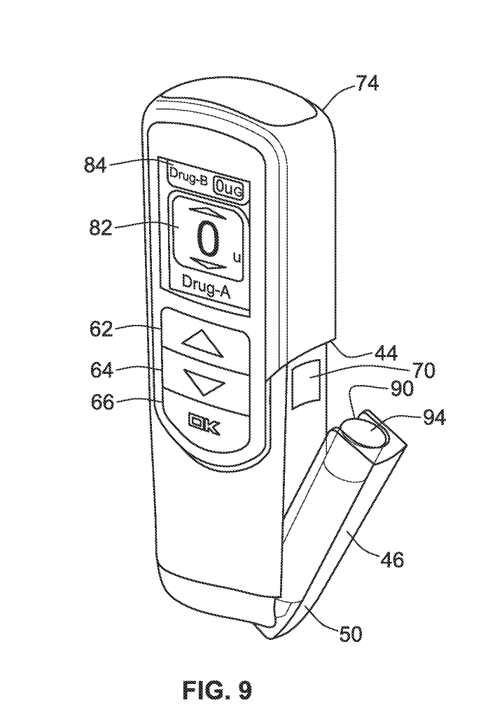

In one arrangement, the first and a second cartridge retainers 50, 52 comprise hinged cartridge retainers. These hinged retainers allow user access to the cartridges. For example, FIG. 9 illustrates a perspective view of the cartridge holder 40 illustrated in FIG. 2 with the first hinged cartridge retainer 50 in an open position. FIG. 9 illustrates how a user might access the first cartridge 90 by opening up the first retainer 50 and thereby having access to the first cartridge 90. A user might access the second cartridge 100 contained in the second hinged retainer 52 in a similar manner. Of course, if different sized cartridges are used, a user might access the second cartridge 100 in a different manner.

As illustrated in FIGS. 9 and 10, the drug delivery device 10 may comprise a cartridge detection system. Such a system may be used so as to confirm that the cartridge 90 has been properly inserted into the first cartridge retainer 50. The cartridge detection device 70 is provided along an inner portion of the cartridge holder 40. An alternative location of the detection device may also be used.

In one arrangement, the first or primary cartridge 90 containing first medicament and the second or secondary cartridge 100 containing the second medicament are of similar dimensions. In another arrangement, the first cartridge 90 is a different size than the second cartridge 100. As just one example, the first medicament (e.g., a long acting insulin) could be provided within a 3 ml cartridge and this cartridge loaded into the first retainer 50. In addition, the second medicament (e.g., a GLP1) may be provided within a shortened 1.7 ml cartridge and could be loaded into the second retainer 52. Because the second hinged retainer contains a smaller sized cartridge, the second retainer would be sized differently than the first retainer. Accordingly, in this arrangement, the primary cartridge retainer 50 is designed to accept a 3 ml cartridge of insulin and the secondary retainer 52 is designed to accept a 1.7 ml cartridge of a GLP1. However, those of skill in the art will readily recognize, alternative cartridge holder structures and cartridge configurations could also be used.

In one arrangement, the cartridge holder 40 includes a cartridge dedication or coding system, such as a mechanical or an electronic cartridge dedication or coding system. Such a system would help to ensure that only a correctly coded cartridge and therefore the correct medicament could be loaded into each cartridge retainer. For instance, an electronic coding system that is able to detect a drug type, expiry date or other similar information could be used. In such an electronic system, the microprocessor control unit could be programmed so that only a properly coded cartridge (and therefore the proper medicaments) would be acceptable in such a system. In such a coded system, the control unit could be programmed with an electronic lock-out so as to lock out or disable the operator interface if an improperly coded cartridge was detected. Preferably, if such an incorrect cartridge were loaded, an error message would be displayed on the digital display 80 so as to notify the user that an incorrect cartridge (and therefore perhaps an incorrect medicament) had been loaded. Most preferably, if such an incorrect cartridge were loaded, the drug delivery device 10 could be programmed so as to lockout the user interface keys and prevent the user from setting a dose.

FIG. 10 illustrates one type of cartridge identification system 110 that may be used with the cartridge housing of drug delivery device 10. For example, FIG. 10 illustrates a cartridge 120 (similar to either the first or the second cartridge 90, 100) residing in a cartridge retainer 116 of a cartridge holder 118. Cartridge retainer 116 may be similar to the cartridge retainers 50, 52 illustrated in FIGS. 3 and 6. A cartridge 120 is illustrated as being nested within an internal cavity of the cartridge retainer 116. A label 122 is provided along an outer surface of the cartridge 120 and a bar code 124 is provided along a portion of this label 122.

In FIG. 10, the cartridge identification system 110 comprises a one dimensional (“1D”) bar code reading system. In such a cartridge identification system 110, the barcode is provided along the cartridge surface and this bar code is an optical machine-readable representation of certain information. Alternatively, a two dimensional bar code reader could also be used. In such an arrangement, patterns of squares, dots, hexagons and other geometric patterns within images may be provided either on the cartridge outer surface itself or on a cartridge label. In addition to or instead of a bar code reader, a cartridge detection device 70 may be provided along an inner surface wall of the system 110.

As just one example, the cartridge holder 118 may comprise a bar code reader 126. In one arrangement, this reader could comprise a 1D bar code reader comprising a light source 128 and a photo diode 130 and these two elements could be provided along an inner surface of the cartridge housing 118 adjacent the cartridge retainer 116. As illustrated, the light source 128 and a photo diode 130 may placed next to each other and directed towards the barcode on the cartridge. To read the bar code 124 provided on the label 122 of the cartridge 120, the light source 128 illuminates various lines provided on the label 122 as the cartridge is inserted into the cartridge housing 118. This light is then reflected and the photo diode 130 measures the intensity of the light reflected back from the light source 128 and a waveform is generated. The micro-processor coupled to this cartridge identification system 110 uses this generated waveform to measure the widths of the bars and spaces of the bar code 124. For example, dark bars in the bar code absorb the illuminated light while the white spaces reflect light.

As such, the voltage waveform generated by the photo diode will represent a duplicate of the bar and space pattern in the bar code. This waveform is then decoded by an algorithm provided in the micro-processor. Alternatively, a 2D barcode reader could also be used. One advantage of such a reader is that relative motion between the cartridge and the cartridge holder would not be required.

Utilizing such cartridge identification in Applicants' proposed drug delivery device 10 results in certain advantages. For example, such a cartridge identification arrangement can provide a method of retrieving information from the cartridges to determine the manufacturer or supplier of the cartridge. Such a system could also determine the type of medicament contained within the cartridge and then may also determine information relating to the drug contained within the cartridge. For example, the cartridge identification system could determine whether the cartridge that was inserted into the first retainer that is supposed to contain the primary medicament actually comprises a cartridge containing such a primary medicament. Such an identification scheme could comprise either a passive or active type of identification scheme. For example, it could comprise a passively (typically mechanical) or active (typically electrical) identification scheme. Such cartridge identification schemes may comprise identification through a microchip interface or through a radio frequency identification (RF-ID) interface. The cartridge may then comprise a readable memory comprising information about the cartridge. The memory may also be writeable, for example to store information on the used number of units, or information on an estimated remaining content in the cartridge and the date first used. The remaining content may be given in number of units, mg, ml and/or the like. The information on the remaining content may be updated when content has been expelled from the cartridge.

In one arrangement, the cartridge holder 40 may be provided as a disposable cartridge holder. For example, in such an arrangement, a medical device supplier or a medicament supplier could supply the cartridge holder containing the two medicaments and these would not be replaceable by the end user. Therefore, once either the primary or secondary medicament of such a cartridge holder has been expended, the entire cartridge holder is removed from the drug dispensing portion of the drug delivery device and is discarded. Thereafter, the user or patient could then attach a new cartridge holder containing two fresh cartridges to the drug dispensing portion of the drug delivery device.

The disposable nature of such a cartridge holder would provide a number of advantages. For example, such a cartridge holder would help to prevent inadvertent medicament cross use: that is, using an incorrect primary or secondary medicament within the cartridge housing. Such an arrangement could also help prevent tampering of the medicaments and could also help eliminate counterfeit products from being used with the drug delivery device. In addition, the cartridge holder may be connected to the device main body where the device main body comprises a one dimensional (“1D”) bar code reading system. Such a coding system could comprise a system similar to the coding system 110 discussed above.

As mentioned above when discussing FIGS. 2 and 3, an interface 200 is coupled to the distal end 15 of the cartridge holder 40. FIG. 11 illustrates a flat view of the interface 200 unconnected to the distal end of the cartridge holder 40. As noted above, the distal end of the interface 200 is configured to engage a medicated module. Such engagement is made possible by the threaded connecting means 216 of the interface 200.

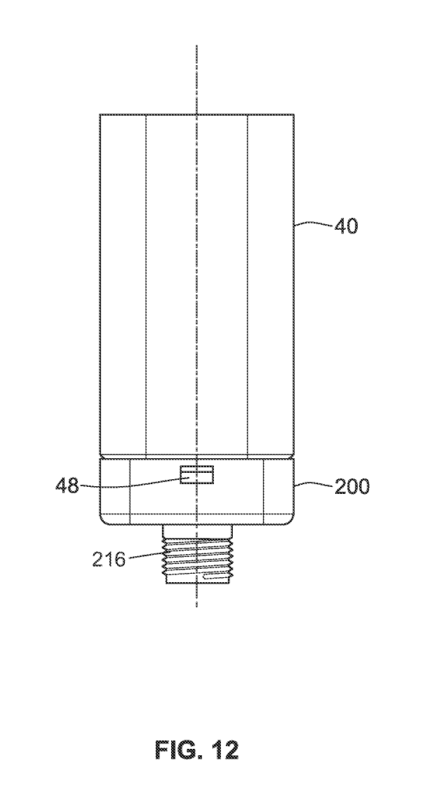

In FIG. 12, the interface 200 illustrated in FIG. 11 is shown coupled to the cartridge holder 40. The axial attachment means between the interface 200 and the cartridge holder 40 can be any known axial attachment means to those skilled in the art, including snap locks, snap fits, snap rings, keyed slots, and combinations of such connections. The connection or attachment between the interface and the cartridge holder may also contain additional features (not shown), such as connectors, stops, splines, ribs, grooves, pips, clips and the like design features, that ensure that specific hubs are attachable only to matching drug delivery devices.

Referring now to FIGS. 11-12 and 13-18, one arrangement of interface 200 will now be discussed. In this arrangement, interface 200 comprises:
a. a main outer body 210,
b. an first inner body 220,
c. a second inner body 230,
d. a first piercing needle 240,
e. a second piercing needle 250,
f. a valve seal 260, and
g. a septum 270.

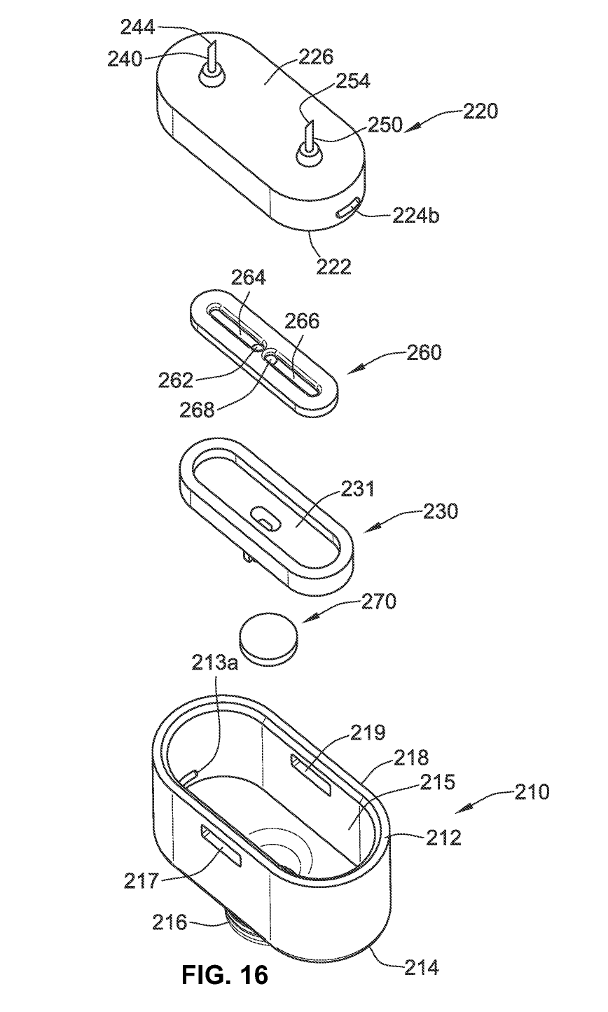

The main outer body 210 comprises a main body proximal end 212 and a main body distal end 214. At the proximal end 212 of the outer body 210, a connecting member is configured so as to allow the interface 200 to be attached to the distal end of the cartridge holder 40. The connecting member may be configured to allow the interface 200 to be removably connected the cartridge holder 40. In one interface arrangement, the proximal end of the interface 200 is configured with an upwardly extending wall 218 having at least one recess. For example, as may be seen from FIGS. 14 and 16, the upwardly extending wall 218 comprises at least a first recess 217 and a second recess 219.

The first and the second recesses 217, 219 are positioned within this main outer body wall so as to cooperate with an outwardly protruding member located near the distal end of the cartridge housing 40 of the device 10. For example, this outwardly protruding member 48 of the cartridge housing may be seen in FIGS. 11 and 12. A second similar protruding member is provided on the opposite side of the cartridge housing. As such, when the interface 200 is axially slid over the distal end of the cartridge housing 40, the outwardly protruding members will cooperate with the first and second recess 217, 219 to form an interference fit, form fit, or snap lock. Alternatively, and as those of skill in the art will recognize, any other similar connection mechanism that allows for the interface and the cartridge housing 40 to be axially coupled could be used as well.

The main outer body 210 and the distal end of the cartridge holder 40 act to form an axially engaging snap lock or snap fit arrangement that could be axially slid onto the distal end of the cartridge housing. In one alternative arrangement, the interface 200 may be provided with a coding feature so as to prevent inadvertent interface cross use. That is, the inner body of the hub could be geometrically configured so as to prevent an inadvertent cross use of one or more interfaces.

A mounting hub 216 is provided at a distal end 214 of the main outer body 210 of the interface hub 200. Such a mounting hub can be configured to be releasably connected to a medicated module. As just one example, this connecting means 216 may comprise an outer thread that engages an inner thread provided along an inner wall surface of a hub of a medicated module, such as the exemplary medicated modules described in detail below and shown in FIGS. 51-59 Alternative releasable connectors may also be provided such as a snap lock, a snap lock released through threads, a bayonet lock, a form fit, or other similar connection arrangements.

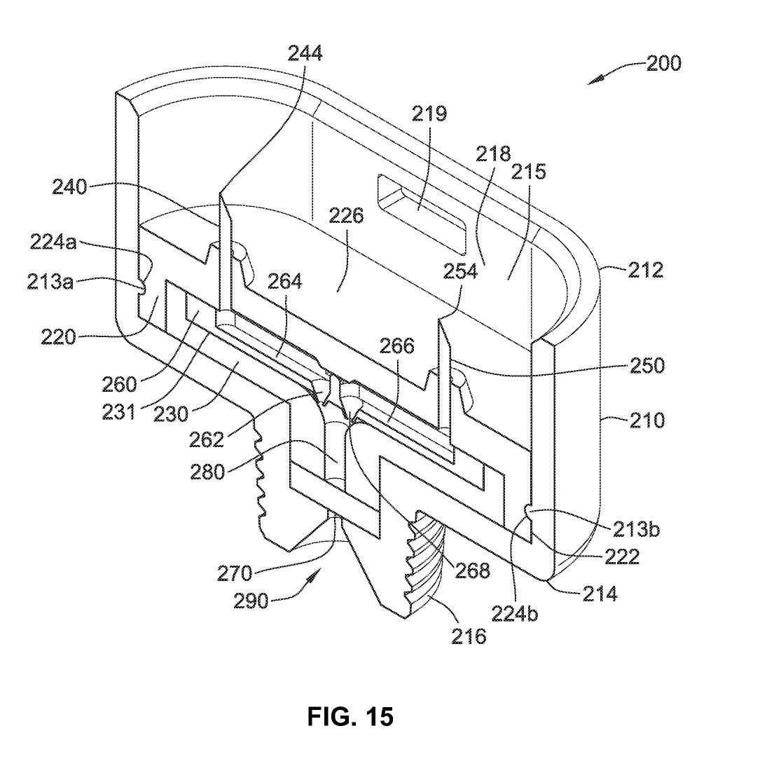

As illustrated in FIGS. 14-18, the first inner body 220 is coupled to an inner surface 215 of the extending wall 218 of the main outer body 210. This first inner body 220 may be coupled by way of a rib and groove form fit arrangement to an inner surface of the outer body 210. For example, as can be seen from FIG. 15, the extending wall 218 of the main outer body 210 is provided with a first rib 213a and a second rib 213b. This first rib 213a is also illustrated in FIG. 16. These ribs 213a and 213b are positioned along the inner surface 215 of the wall 218 of the outer body 210 and create a form fit or snap lock engagement with cooperating grooves 224a and 224b of the first inner body 220. In a preferred arrangement, these cooperating grooves 224a and 224b are provided along an outer surface 222 of the first inner body 220.

In addition, as can be seen in FIGS. 14-17, a proximal surface 226 near the proximal end of the first inner body 220 may be configured with at least a first proximally positioned piercing needle 240 comprising a proximal piercing end portion 244. Similarly, the first inner body 220 is configured with a second proximally positioned piercing needle 250 comprising a proximally piercing end portion 254. Both the first and second needles 240, 250 are rigidly mounted on the proximal surface 226 of the first inner body 220.

The interface 200 may also comprise a valve arrangement. Such a valve arrangement could be constructed so as to prevent cross contamination of the first and second medicaments contained in the first and second reservoirs, respectively. The valve arrangement may also be configured so as to prevent back flow and cross contamination of the first and second medicaments.

In the example shown in FIGS. 15-17, interface 200 includes a valve arrangement in the form of a valve seal 260. Such a valve seal 260 may be provided within a cavity 231 defined by the second inner body 230, so as to form a holding chamber 280. Preferably, cavity 231 resides along an upper surface of the second inner body 230. This valve seal comprises an upper surface that defines both a first fluid groove 264 and second fluid groove 266. For example, FIG. 15 illustrates the position of the valve seal 260, seated between the first inner body 220 and the second inner body 230.

During an injection step, this seal valve 260 helps to prevent the primary medicament in the first pathway from migrating to the secondary medicament in the second pathway while also preventing the secondary medicament in the second pathway from migrating to the primary medicament in the first pathway. As shown, the valve seal 260 comprises a first non-return valve 262 and a second non-return valve 268. As such, the first non-return valve 262 prevents fluid transferring along the first fluid pathway 264, for example a groove in the seal valve 260, from returning back into this pathway 264. Similarly, the second non-return valve 268 prevents fluid transferring along the second fluid pathway 266 from returning back into this pathway 266.

Together, the first and second grooves 264, 266 converge towards the non-return valves 262 and 268 respectively, to then provide for an output fluid path or a holding chamber 280. This holding chamber 280 is defined by an inner chamber defined by a distal end of the second inner body both the first and the second non return valves 262, 268 along with a pierceable septum 270. As illustrated, this pierceable septum 270 is positioned between a distal end portion of the second inner body 230 and an inner surface defined by the hub 216 of the main outer body 210.

The holding chamber 280 terminates at an outlet port of the interface 200. This outlet port 290 is preferably centrally located in the hub 216 of the interface 200 and assists in maintaining the pierceable seal 270 in a stationary position. As such, when a medicated module is attached to the hub 216 of the interface 200, the outlet port 290 allows both medicaments to be in fluid communication with the attached medicated module.

The interface hub 200 further comprises a second inner body 230. As can be seen from FIG. 15, this second inner body 230 has an upper surface that defines a recess, and the valve seal 260 is positioned within this recess. Therefore, when the interface 200 is assembled as shown in FIG. 15, the second inner body 230 will be positioned between a distal end of the outer body 210 and the first inner body 220. Together, second inner body 230 and the main outer body hold the septum 270 in place. The distal end of the inner body 230 may also form a cavity or holding chamber that can be configured to be fluid communication with both the first groove 264 and the second groove 266 of the valve seal.

Although not shown, the interface 200 could be supplied by a manufacturer as being contained in a protective and sterile capsule or container. As such, where the user would peel or tear open a seal or the container itself to gain access to the sterile single interface. In some instances it might be desirable to provide two or more seals for each end of the interface. The seal may allow display of information required by regulatory labeling requirements. When a disposable medicated module is used as a single dispense assembly to deliver the combination dose, it is preferred that the interface is designed to be economical and safe for allowing the user to attach a new medicated module for each injection.

Axially sliding the main outer body 210 over the distal end of the drug delivery device attaches the interface 200 to the multi-use auto-injector device. In this manner, a fluid communication may be created between the first needle 240 and the second needle 250 with the primary medicament of the first cartridge and the secondary medicament of the second cartridge, respectively.

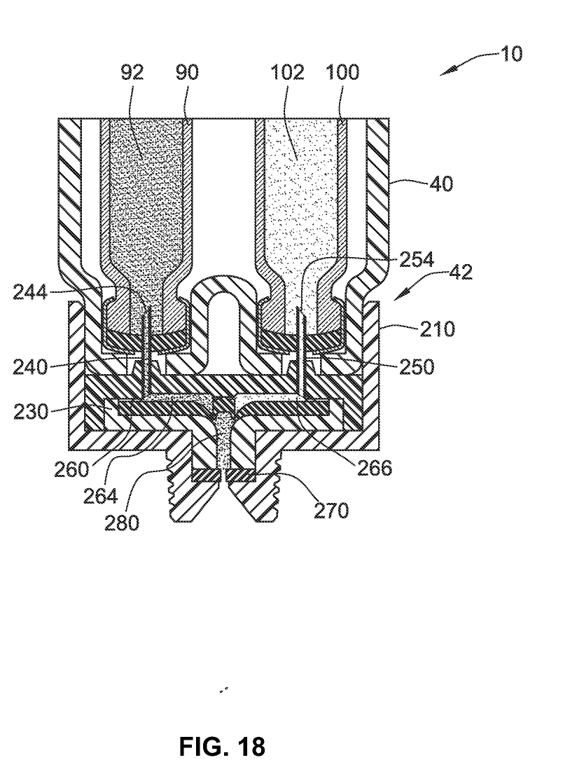

FIG. 18 illustrates the interface 200 after it has been mounted onto the distal end 42 of the cartridge holder 40 of the drug delivery device 10 illustrated in FIG. 1. The cartridge holder 40 is illustrated as having a first cartridge containing a first medicament and a second cartridge containing a second medicament.

When the interface 200 is first mounted over the distal end of the cartridge holder 40, the proximal piercing end 244 of the first piercing needle 240 pierces the septum of the first cartridge 90 and thereby resides in fluid communication with the primary medicament 92 of the first cartridge 90. A distal end of the first piercing needle 240 will also be in fluid communication with a first fluid path groove 264 defined by the valve seal 260.

Similarly, the proximal piercing end 254 of the second piercing needle 250 pierces the septum of the second cartridge 100 and thereby resides in fluid communication with the secondary medicament 102 of the second cartridge 100. A distal end of this second piercing needle 250 will also be in fluid communication with a second fluid path groove 266 defined by the valve seal 260.

FIG. 18 illustrates one arrangement of the interface 200 when it is coupled to a distal end 15 of the main body 14 of drug delivery device 10. The interface 200 may be removably coupled to the cartridge holder 40 of the drug delivery device 10, thus allowing the user to replace the interface 200 after a desired number of uses.

As illustrated in FIG. 18, the interface 200 is coupled to the distal end of a cartridge housing 40. This cartridge holder 40 is illustrated as containing the first cartridge 90 containing the primary medicament 92 and the second cartridge 100 containing the secondary medicament 102. Once coupled to the cartridge housing 40, the interface 200 essentially provides a mechanism for providing a fluid communication path from the first and second cartridges 90, 100 to the common holding chamber 280.

In one arrangement, the interface 200 is configured so that it attaches to the main body in only one orientation. As such, once the interface 200 is attached to the cartridge holder 40, the primary needle 240 can only be used for fluid communication with the primary medicament 92 of the first cartridge 90 and the interface 200 would be prevented from being reattached to the holder 40 so that the primary needle 240 could be used for fluid communication with the secondary medicament 102 of the second cartridge 100. Such a one-way orientation connecting mechanism may help to reduce potential cross contamination between the two medicaments 92 and 102.

In one arrangement, the drug delivery device 10 comprises a detection sensor so as to sense or confirm that the interface 200 has been correctly mounted onto the cartridge housing 40. Such a detection sensor may comprise either a mechanical, an electrical, a capacitive, an inductive or other similar type sensor. This sensor may be provided near the distal end of the cartridge housing.

In addition, the drug delivery device may comprise a similar detection sensor for detecting the presence of a medicated module. For example, such a sensor may be provided adjacent the needle hub of the interface 200. Preferably, either or both of the detection sensors would be communicatively coupled to the micro-processor.

Optionally, the micro-processor would be programmed so as prevent a user from setting a dose with the drug delivery device 10 unless the device has detected that both the interface 200 has been properly mounted to the cartridge holder 40 and that a medicated module has been properly mounted onto the interface. If either the interface or the medicated module has been detected as being incorrectly mounted, the user may be locked out of the device and a connection error may be shown on the digital display 80.

Additionally, the interface 200 could incorporate a safety shield device (in addition to the guard of the medicated module) that would prevent accidental needle sticks and reduce the anxiety experienced by users who suffer from needle phobia. The exact design of the safety shield is not critical to the presently described auto-injector device and system. In one arrangement, activation of the safety shield could unlock the drug delivery system or enable medicament to be dispensed via the interface and medicated module.

In one arrangement, the interface 200 is a disposable interface and as such, the interface 200 is discarded when either the first or the second cartridge in the device is replaced (e.g., when such cartridge is empty). In one arrangement, the interface 200 may be provided in a drug delivery kit. For example, in one drug delivery kit arrangement, an interface can be provided with each replacement cartridge. The interface 200 may also be a multi-use interface.

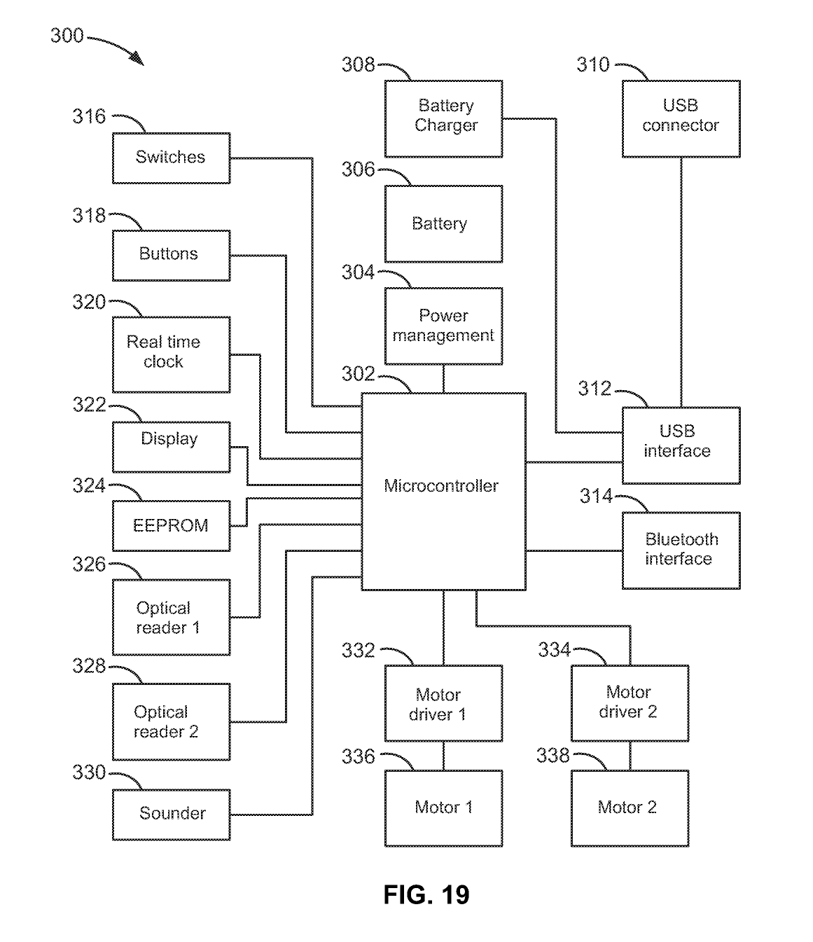

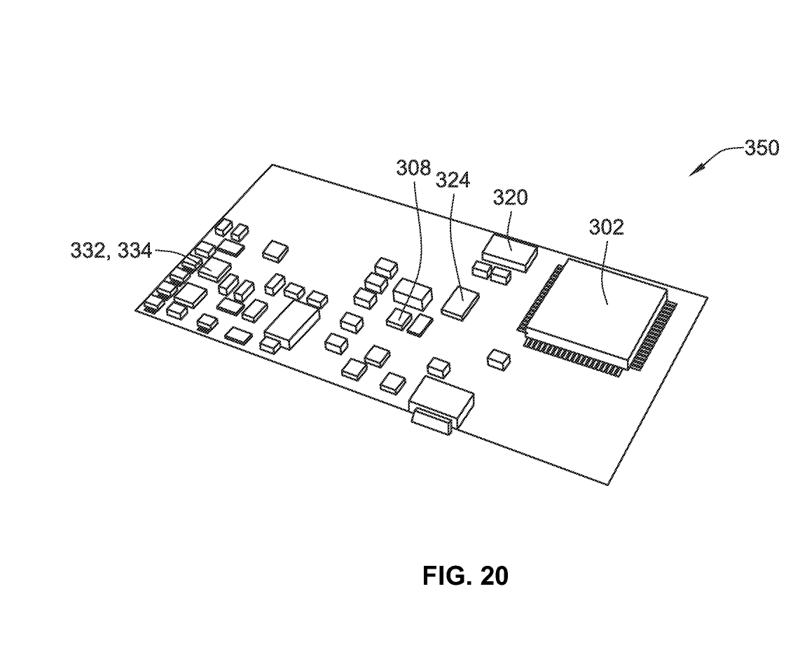

FIG. 19 illustrates a functional block diagram of a control unit to operate and control the drug delivery device illustrated in FIG. 1. FIG. 20 illustrates one arrangement of a printed circuit board (PCB) or printed circuit board assembly (PCBA) 350 that may comprise certain portions of the control unit illustrated in FIG. 19.

Referring now to both FIGS. 19 and 20, it may be seen that the control unit 300 comprises a microcontroller 302. Such a microcontroller may comprise a Freescale MCF51JM microcontroller. The microcontroller is used to control the electronic system for the drug delivery device 10. It includes internal analogue to digital converters and general purpose digital I/O lines. It can output digital Pulse Width Modulated (PWM) signals. It includes an internal USB module. In one arrangement, a USB protection circuit such as ON-Semi NUP3115 may be implemented. In such an implementation, the actual USB communications may be provided on board the microcontroller 302.

The control unit further comprises a power management module 304 coupled to the microcontroller 302 and other circuit elements. The power management module 304 receives a supply voltage from a main power source such as the battery 306 and regulates this supply voltage to a plurality of voltages required by other circuit components of the control unit 300. In one preferred control unit arrangement, switched mode regulation (by means of a National Semiconductor LM2731) is used to step up the battery voltage to 5V, with subsequent linear regulation to generate other supply voltages required by the control unit 300.

The battery 306 provides power to the control unit 300 and is preferably supplied by a single lithium-ion or lithium-polymer cell. This cell may be encapsulated in a battery pack that contains safety circuitry to protect against overheating, overcharging and excessive discharge. The battery pack may also optionally contain coulomb counting technology to obtain an improved estimate of remaining battery charge.

A battery charger 308 may be coupled to the battery 306. One such battery charger may be based on Texas Instruments (TI) BQ24150 along with other supporting software and hardware modules. In one preferred arrangement, the battery charger 308 takes energy from an external wired connection to the drug delivery device 10 and uses it to charge the battery 306. The battery charger 308 can also be used to monitor the battery voltage and charge current to control battery charging. The battery charger 308 can also be configured to have bidirectional communications with the microcontroller 302 over a serial bus. The charge status of the battery 306 may be communicated to the microcontroller 302 as well. The charge current of the battery charger may also be set by the microcontroller 302.

The control unit may also comprise a USB connector 310. A micro USB-AB connector may be used for wired communications and to supply power to the device.

The control unit may also comprise a USB interface 312. This interface 312 may be external to the microcontroller 302. The USB interface 312 may have USB master and/or USB device capability. The USB interface 312 may also provide USB on-the-go functionality. The USB interface 312 external to the microcontroller also provides transient voltage suppression on the data lines and VBUS line.

An external Bluetooth interface 314 may also be provided. The Bluetooth interface 314 is preferably external to the microcontroller 302 and communicates with this controller 302 using a data interface.

Preferably, the control unit further comprises a plurality of switches 316. In the illustrated arrangement, the control unit 300 may comprise eight switches 316 and these switches may be distributed around the device. These switches 316 may be used to detect and or confirm at least the following:
a. Whether the interface 200 has been properly attached to the drug delivery device 10;
b. Whether the removable cap 18 has been properly attached to the main body 20 of the drug delivery device 10;
c. Whether the first cartridge retainer 50 of the cartridge holder 40 for the first cartridge 90 has been properly closed;
d. Whether the second cartridge retainer 52 of the cartridge holder 40 for the second cartridge 100 has been properly closed;
e. To detect the presence of the first cartridge 90;
f. To detect the presence of the second cartridge 100;
g. To determine the position of the stopper 94 in the first cartridge 90; and
h. To determine the position of the stopper 104 in the second cartridge 100.

These switches 316 are connected to digital inputs, for example to general purpose digital inputs, on the microcontroller 302. Preferably, these digital inputs may be multiplexed in order to reduce the number of input lines required. Interrupt lines may also be used appropriately on the microcontroller 302 so as to ensure timely response to changes in switch status.

In addition, and as described in greater detail above, the control unit may also be operatively coupled to a plurality of human interface elements or push buttons 318. In one preferred arrangement, the control unit 300 comprises eight push buttons 318 and these are used on the device for user input for the following functions:
a. Dose dial up;
b. Dose dial down;
c. Sound level;
d. Dose;
e. Eject;
f. Prime;
g. Dose set; and
h. OK.

These buttons 318 are connected to digital inputs, for example to general purpose digital inputs, on the microcontroller. Again, these digital inputs may be multiplexed so as to reduce the number of input lines required. Interrupt lines will be used appropriately on the microcontroller to ensure timely response to changes in switch status. In an example embodiment, the function of one or more buttons may be replaced by a touch screen.

In addition, the control unit 300 comprises a real time clock 320. Such a real time clock may comprise an Epson RX4045 SA. The real-time clock 320 may communicate with the microcontroller 302 using a serial peripheral interface or similar.

A digital display module 322 in the device preferably uses LCD or OLED technology and provides a visual signal to the user. The display module incorporates the display itself and a display driver integrated circuit. This circuit communicates with the microcontroller 302 using a serial peripheral interface or parallel bus.

The control unit 300 also comprises a memory device, for example volatile and non-volatile memory. Volatile memory may be random access memory (RAM), for example static RAM or dynamic RAM and/or the like, as working memory of microcontroller 302. Non-volatile memory may be read only memory (ROM), FLASH memory or electrically erasable programmable read-only memory (EEPROM), such as an EEPROM 324. Such an EEPROM may comprise an Atmel AT25640. The EEPROM may be used to store system parameters and history data. This memory device 324 communicates with the processor 302 using a serial peripheral interface bus.

The control unit 300 further comprises a first and a second optical reader 326, 328. Such optical readers may comprise Avago ADNS3550. These optical readers 326, 328 may be optional for the drug delivery device 10 and are, as described above, used to read information from a cartridge when such a cartridge is inserted into either the first or the second cartridge retainers 50, 52. Preferably, a first optical reader is dedicated for the first cartridge and the second optical reader is dedicated for the second cartridge. An integrated circuit designed for use in optical computer mice may be used to illuminate a static 2D barcode on the drug cartridge, positioned using a mechanical feature on the drug cartridge, and read the data it contains. This integrated circuit may communicate with the microcontroller 302 using a serial peripheral interface bus. Such a circuit may be activated and deactivated by the microcontroller 302 e.g., to reduce power consumption when the circuit is not needed, for example by extinguishing the cartridge illumination when data is not being read.

As previously mentioned, a sounder 330 may also be provided in the drug delivery device 10. Such a sounder may comprise a Star Micronics MZT03A. Applicants' proposed sounder may be used to provide an audible signal to the user. The sounder 330 may be driven by a pulse-width modulation (PWM) output from the microcontroller 302. In an alternative configuration, the sounder may play polyphonic tones or jingles and play stored voice commands and prompts to assist the user in operating or retrieving information from the device.

The control unit 300 further comprises a first motor driver 332 and a second motor driver 334. The motor drive circuitry may comprise Freescale MPC17C724 and is controlled by the microcontroller 302. For example, where the motor drive comprises a stepper motor drive, the drive may be controlled using general purpose digital outputs. Alternatively, where the motor drive comprises a brushless DC motor drive, the drive may be controlled using a Pulse Width Modulated (PWM) digital output. These signals control a power stage, which switches current through the motor windings. The power stage requires continuous electrical commutation. This may for example increase device safety, decreasing the probability of erroneous drug delivery.

The power stage may consist of a dual H-bridge per stepper motor, or three half-bridges per brushless DC motor. These may be implemented using either discrete semiconductor parts or monolithic integrated circuits.

The control unit 300 further comprises a first and a second motor 336, 338, respectively. As explained in greater detail below, the first motor 336 may be used to move the stopper 94 in the first cartridge 90. Similarly, the second motor 338 may be used to move the stopper 104 in the second cartridge. The motors can be stepper motors, brushless DC motors, or any other type of electric motor. The type of motor may determine the type of motor drive circuit used. The electronics for the device may be implemented with one main, rigid printed circuit board assembly, potentially with additional smaller flexible sections as required, e.g., for connection to motor windings and switches.

The micro-processor provided on the PCBA 350 will be programmed to provide a number of features and carry out a number of calculations. For example, and perhaps most importantly, the micro-processor will be programmed with an algorithm for using a certain therapeutic dose profile to calculate at least a dose of the secondary medicament based at least in part on the selected dose of the primary medicament. For such a calculation, the controller may also analyze other variables or dosing characteristics in calculating the amount of second medicament to administer. For example, other considerations could include at least one or more of the following characteristics or factors:
a. Time since last dose;
b. Size of last dose;
c. Size of current dose;
d. Current blood glucose level;
e. Blood glucose history;
f. Maximum and/or minimum permissible dose size;
g. Time of day;
h. Patient's state of health;
i. Exercise taken; and
j. Food intake.

These parameters may also be used to calculate the size of both the first and the second dose size.

In one arrangement, and as will be described in greater detail below, a plurality of different therapeutic dose profiles may be stored in the memory device or devices operatively coupled to the micro-processor. In an alternative arrangement, only a single therapeutic dose profile is stored in the memory device operatively coupled to the micro-processor.

The presently proposed electro-mechanical drug delivery device is of particular benefit to patients with dexterity or computational difficulties. With such a programmable device, the single input and associated stored predefined therapeutic profile removes the need for the user or patient to calculate their prescribed dose every time they use the device. In addition, the single input allows easier dose setting and dispensing of the combined compounds.

In addition to computing the dose of the second medicament, the micro-processor can be programmed to achieve a number of other device control operations. For example, the micro-processor may be programmed so as to monitor the device and shut down the various elements of the system to save electrical energy when the device is not in use. In addition, the controller can be programmed to monitor the amount of electrical energy remaining in the battery 306. In one preferred arrangement, an amount of charge remaining in the battery can be indicated on the digital display 80 and a warning may be given to the user when the amount of remaining battery charge reaches a predetermined threshold level. In addition, the device may include a mechanism for determining whether there is sufficient power available in the battery 306 to deliver the next dose, or it will automatically prevent that dose from being dispensed. For example, such a monitoring circuit may check the battery voltage under different load conditions to predict the likelihood of the dose being completed. In a preferred configuration the motor in an energized (but not moving) condition and a not energized condition may be used to determine or estimate the charge of the battery.

The drug delivery device 10 may be configured to communicate via a data link (i.e., either wirelessly or hard wired) with various computing devices, such as a desktop or laptop computer. For example, the device may comprise a Universal Serial Bus (USB) for communicating with a PC or other devices. Such a data link may provide a number of advantages. For example, such a data link may be used to allow certain dose history information to be interrogated by a user. Such a data link could also be used by a health care professional to modify certain key dose setting parameters such as maximum and minimum doses, a certain therapeutic profile, etc. The device may also comprise a wireless data link, for example an IRDA data link or a Bluetooth data link. A preferred Bluetooth module comprises a Cambridge Silicon Radio (CSR) Blue core 6. In an example embodiment, the device has USB On-The-Go (USB OTG) capability. USB OTG may allow the drug delivery device 10 to generally fulfill the role of being slave to a USB host (e.g., to a desktop or notebook computer) and to become the host themselves when paired with another slave device (e.g. a BGM).

For example, standard USB uses a master/slave architecture. A USB Host acts as the protocol master, and a USB ‘Device’ acts as the slave. Only the Host can schedule the configuration and data transfers over the link. The Devices cannot initiate data transfers, they only respond to requests given by a host. Use of OTG in Applicants' drug delivery device 10 introduces the concept that the drug delivery device can switch between the master and slave roles. With USB OTG, Applicants' device 10 at one time be a ‘Host’ (acting as the link master) and a ‘Peripheral’ (acting as the link slave) at another time.

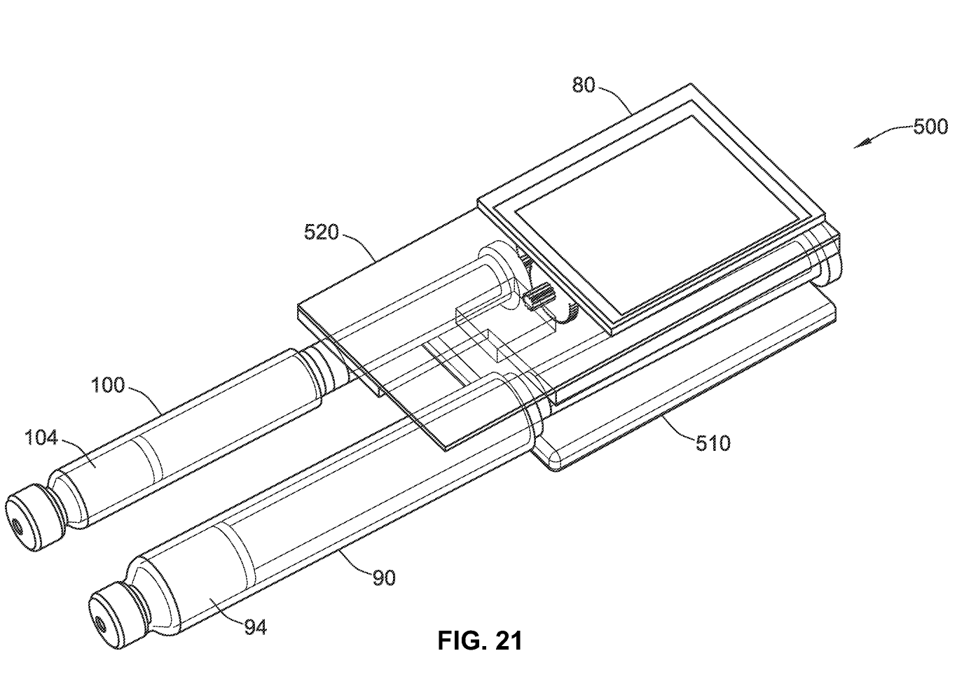

FIG. 21 illustrates various internal components of the auto-injector drug delivery device 10 illustrated in FIGS. 1a and 1b including one arrangement of a drive train 500. As illustrated, FIG. 21 illustrates the digital display 80, a printed circuit board assembly (PCBA) 520 (such as the PCB 350 illustrated in FIG. 20), along with a power source or battery 510. The PCBA 520 may be positioned between the digital display 80 and a drive train 500 with the battery or power source 510 positioned beneath this drive train. The battery or power source 510 is electronically connected to provide power to the digital display 80, the PCBA 520 and the drive train 500. As illustrated, both the first and second cartridges 90, 100 are shown in an expended state. That is, the first and second cartridges are illustrated in an empty state having a stopper at a most distal position. For example, the first cartridge 90 (which ordinarily contains the first medicament 92) is illustrated as having its stopper 94 in the distal position. The stopper 104 of the second cartridge 100 (ordinarily containing the second medicament 102) is illustrated in a similar position.

With reference to FIG. 21, it may be seen that there is provided a first region defining a suitable location for a power source 510 such as a replaceable battery or batteries. The power source 510 may comprise a rechargeable power source and may be recharged while the power source 510 remains in the device. Alternatively, the power source 510 may be removed from the drug delivery device 10 and recharged externally, for example, by way of a remote battery charger. This power source may comprise a Lithium-Ion or Lithium-polymer power source. In this preferred arrangement, the battery 510 comprises a generally flat and rectangular shaped power source.

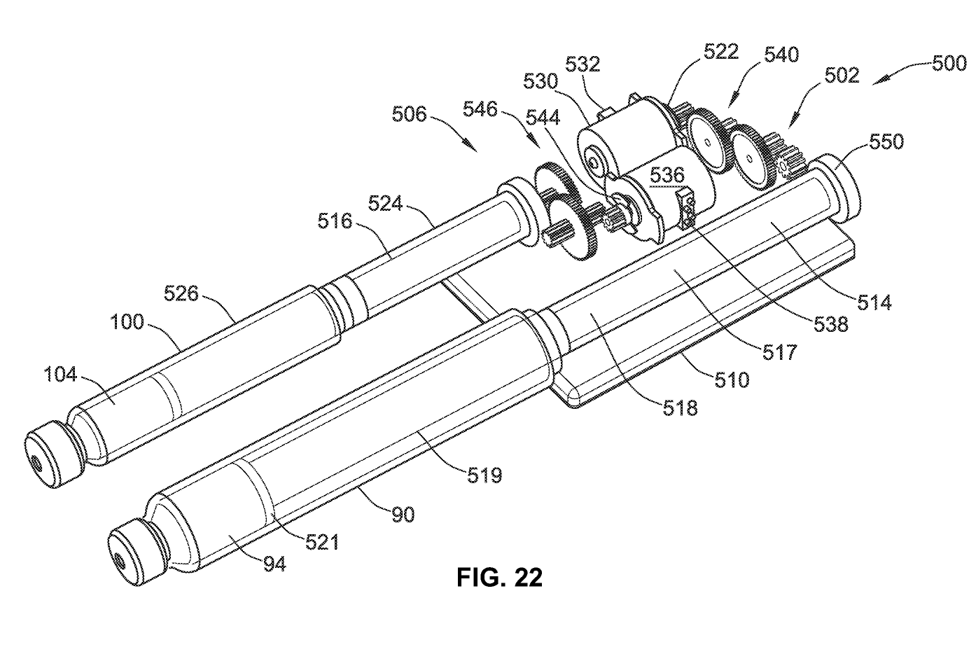

FIG. 22 illustrates the first arrangement of the electro-mechanical system illustrated in FIG. 21 with both the digital display 80 and the PCBA 520 omitted. As illustrated in FIG. 22, the electro-mechanical system 500 operates to expel a dose from the first cartridge 90 containing the primary medicament 92 and the second cartridge 100 containing the secondary medicament 102. Again, as illustrated in FIG. 22, the first and second cartridges 90, 100 are illustrated in an empty state having stoppers at a most distal position.

In this preferred electro-mechanical system 500, the system comprises an independent mechanical driver for each cartridge 90, 100. That is, an independent mechanical driver 502 operates to expel a dose from the first cartridge 90 and an independent mechanical driver 506 operates to expel a dose from the second cartridge 100. In an alternative electro-mechanical system 500 operating on three different medicaments, three independent mechanical drivers could be provided. The independent mechanical drivers act under control of the motor drivers 332, 334 of the control unit 300 (see, e.g., FIG. 19).

The first independent mechanical driver 502 operates to expel a dose from the first cartridge 90. This first driver 502 comprises a first motor 530 that is operatively coupled to a first gearing arrangement 540. To energize this motor 530, a connector 532 is provided as a means of electrically connecting to the motor driver 332. This first gearing arrangement 540 is mechanically linked to a proximal portion of the first telescoping piston rod 514. The first telescoping piston rod 514 is illustrated in a fully extended position having a distal end 521 acting on the stopper 94 of the first cartridge 90.

As this gearing arrangement 540 is driven by the output shaft of the first motor 530, this arrangement 540 rotates the proximal portion 518 of the first telescoping piston rod 514.

As this proximal portion 518 of the piston rod 514 is rotated, the second or distal portion 519 of the piston rod 514 is driven in a distal direction.

Preferably, the proximal portion 518 of the telescope piston rod 514 comprises an external thread 517. This thread 517 engages the distal portion 519 which has in integrated nut comprising a short threaded section at a proximal end of the distal portion 519. This distal portion 519 is prevented from rotating via a key acting in a keyway. Such a keyway may pass through the middle of first telescope 514. Therefore, when the first gearbox arrangement 540 causes rotation of the proximal section 518, rotation of the proximal portion 518 acts upon the distal end 521 to thereby drive the distal portion of telescope piston rod to extend along the longitudinal axis.

Moving in this distal direction, the distal end 521 of the second portion 519 of the piston rod 514 exerts a force on a stopper 94 contained within the first cartridge 90. With this distal end 521 of the piston rod 514 exerting a force on the stopper, the user selected dose of the first medicament 92 is forced out of the cartridge 90 and into an attached interface 200 and consequently out of a dispense interface of a medicated module. A similar injection operation occurs with the second independent driver 506 when the controller first determines that a dose of the second medicament 102 is called for and determines the amount of this dose. As previously mentioned, in certain circumstances, the controller may determine that a dose of the second medicament 102 may not be called for and therefore this second dose would be “set” to a “0” dose.

Preferably, motors 530, 536 comprise motors suitable for electronic commutation. Most preferably, such motors may comprise either a stepper motor or a brushless DC motor. To inject a dose of the primary and secondary medicaments 92, 102, which causes a fixed dose of a medicament contained in an attached medicated module to be delivered, a user will first select a dose of the primary medicament by way of the human interface components on the display 80. (see, e.g., FIGS. 1 and 4). After a dose of the drug from the primary medicament 92 has been selected, the microcontroller will utilize a previously stored algorithm for determining the dose size of a second drug 102 from a second medicament cartridge. This pre-defined algorithm may help to determine at least in part the dose of the second medicament 102 based on a pre-selected therapeutic profile. In one arrangement, these therapeutic profiles are user selectable. Alternatively, these therapeutic profiles may be password protected and selectable only by a person authorized with the password, such a physician or patient care giver. In yet another arrangement, the therapeutic profile may only be set by the manufacture or the supplier of the drug delivery device 10. As such, the drug delivery device 10 may be provided with only one profile.

When the dose sizes of the first and second medicaments have been established, the user can press the injection/delivery button 74 (see e.g., FIG. 4). By pressing this button 74, the motor drivers 332, 334 energize both the first and the second motors 530, 536 to begin the injection process described above.

The piston rods 514, 516 are preferably movable between a first fully withdrawn position (not shown) and a second fully extended portion (as shown in FIGS. 21 and 22). With the piston rods 514, 516 in the withdrawn position, the user will be allowed to open up the respective cartridge retainer and remove an empty cartridge. In one arrangement, an end stop switch may be provided in the main body 14 of the drug delivery device 10 so as to detect when either or both of the piston rods 514, 516 are in a fully withdrawn position. Tripping of the end stop switch may release a catch or other fastening device so as to allow access to the main body for replacement of either cartridge 90, 100.

In one arrangement, both the first and second motors 530, 536 operate simultaneously so as to dispense the user selected dose of the first medicament 92 and the subsequently calculated dose of the second medicament 102 simultaneously. That is, both the first and the second independent mechanical drivers 502, 506 are capable of driving the respective piston rods 514, 516 either at the same or a different time. In this manner, now referring to the interface 200 previously discussed, the first medicament 92 enters the holding chamber 280 of the interface 200 at essentially the same time as the second medicament. One advantage of such an injecting step is that a certain degree of mixing can occur between the first and second medicament 92, 102 prior to actual dose administration.
a. If after an injection, the patient determines that one or more of the cartridges 90, 100 is spent and therefore needs to be exchanged, the patient can follow the following method of cartridge exchange: Remove the medicated module from the interface 200;
b. Remove the interface 200 from the cartridge holder 40 of the device 10;
c. Enable a menu option on the digital display 80 to change the first cartridge 90 and/or the second cartridge 100;
d. Rewind the first and/or the second piston rods 514, 516;
e. The first and/or second cartridge retainer doors will pop open;
f. The user removes the spent cartridge and replaces this spent cartridge with a new cartridge;
g. The reservoir doors may manually be closed;
h. Once the doors are closed, the first and second piston rods 514, 516 advance so that a most distal portion of each rod will meet the stopper of the respective cartridge and will stop advancing when a bung detect mechanism coupled to the micro-processor is activated;
i. The user replaces the interface 200 in the one way manner on the cartridge holder 40;
j. The user can, optionally, connect a new medicated module to the interface 200;
k. The user can, optionally, perform a test shot or a priming step with the device 10; and
l. The user can then set the next dose for a subsequent dose administration step.

One or more of the steps may be performed automatically, for example controlled by microcontroller 302, such as the step of rewinding the first and/or second piston rod. In an alternative arrangement, the controller may be programmed so that the first and the second independent mechanical drivers 502, 506 may be operated to dispense either the first medicament 92 or the second medicament 102 prior to the other medicament. Thereafter, the second or the primary medicament may then be dispensed. In one preferred arrangement, the secondary medicament 102 is dispensed before the primary medicament 92. Regardless of which medicament is dispensed from the auto-injector first, the first dispensed medicament will cause the medicament contained in the medicated module to be delivered by forcing it out of the reservoir of the medicated module.

Preferably, the first and second motors 530, 536 comprise electronic commutation. Such commutation may help to minimise the risk of a motor runaway condition. Such a motor runaway condition could occur with a system comprising a standard brushed motor experiencing a fault. In one embodiment of the motor drive system, a watchdog system may be provided. Such a system has the ability to remove power to either or both of the motors in the event of a software malfunction or a failure of the electronic hardware. To prevent the power from being removed, the correct input from a number of sections of the electronic hardware and/or the microcontroller software will need to be provided. If one of these input parameters is incorrect; power may be removed from the motor.

In addition, preferably both motors 530, 536 may be operated in a reverse direction. This feature may be required in order to allow the piston rods 514, 516 to be moved between a first and a second position.

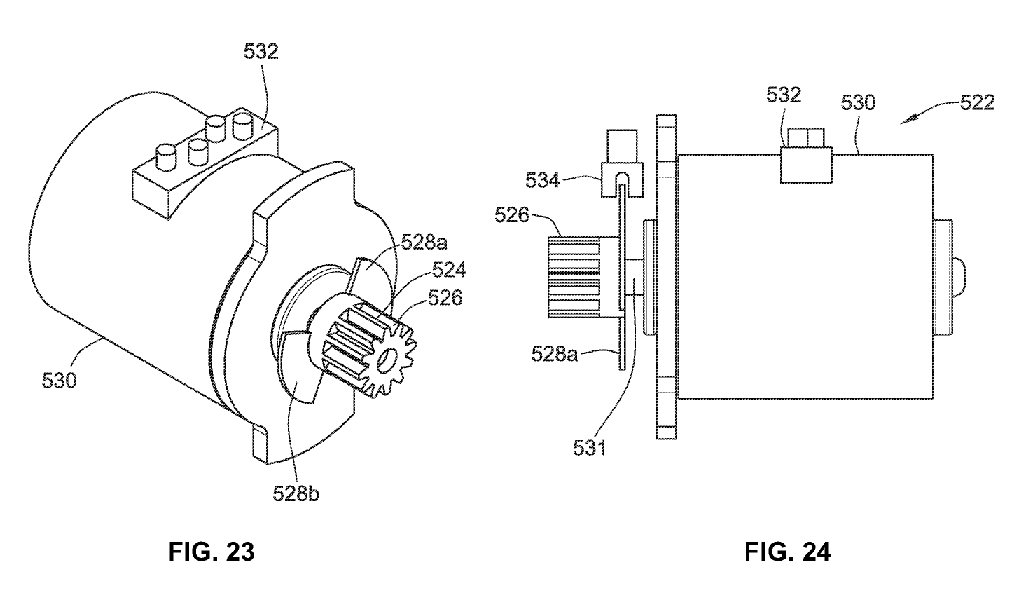

Preferably, the first independent drive train 502 illustrated in FIG. 22 comprises a first motion detection system 522. FIG. 23 illustrates a perspective view of the first motor 530 illustrated in FIG. 22. FIG. 24 illustrates a preferred motion detection system 522 comprising the first motor 530 illustrated in FIG. 23 in conjunction with a digital encoder 534.

As illustrated in FIGS. 23 and 24, such a motion detection system 522 may be beneficial as it can be utilized to provide operational and positional feedback from the first independent driver 502 to the control unit of the drug delivery device 10. For example, with respect to the first independent driver 502, a preferred motion detection system 522 may be achieved through the use of a first motor pinion 524. This first pinion 524 operatively coupled to an output shaft 531 of the first motor 530. The first pinion 524 comprises a rotating gearing portion 526 that drives a first gear of the first gearing arrangement 540 (see, e.g., FIG. 22). The first motor pinion 524 also comprises a plurality of flags 528 a-b. In this first motion detection system arrangement 522, the first pinion 524 comprises a first flag 528a and a second flag 528b. These two flags 528a-b are positioned on the motor pinion 524 so that they pass through a first optical encoder 534 as the motor output shaft 531 and hence the connected first pinion 524 rotate when the motor is driven.

Preferably, as the first and second flags 528a-b pass through the first optical encoder 534, the encoder 534 can send certain electrical pulses to the microcontroller. Preferably, the optical encoder 534 sends two electrical pulses per motor output shaft revolution to the microcontroller. As such, the microcontroller can therefore monitor motor output shaft rotation. This may be advantageous to detect position errors or events that could occur during a dose administration step such as jamming of the drive train, incorrect mounting of a interface or needle assembly such as a medicated module, or where there is a blocked needle.

Preferably, the first pinion 524 comprises a plastic injection molded pinion. Such a plastic injection molded part may be attached to the output motor shaft 531. The optical encoder 534 may be located and attached to a gearbox housing. Such a housing may contain both the first gearing arrangement 540 along with the optical encoder 534. The encoder 534 is preferably in electrical communication with the control unit potentially via a flexible portion of the PCB. In a preferred arrangement, the second independent drive train 506 illustrated in FIGS. 21 and 22 comprises a second motion detection system 544 that operates in a similar fashion as the first motion detection system 522 of the first drive train 502.

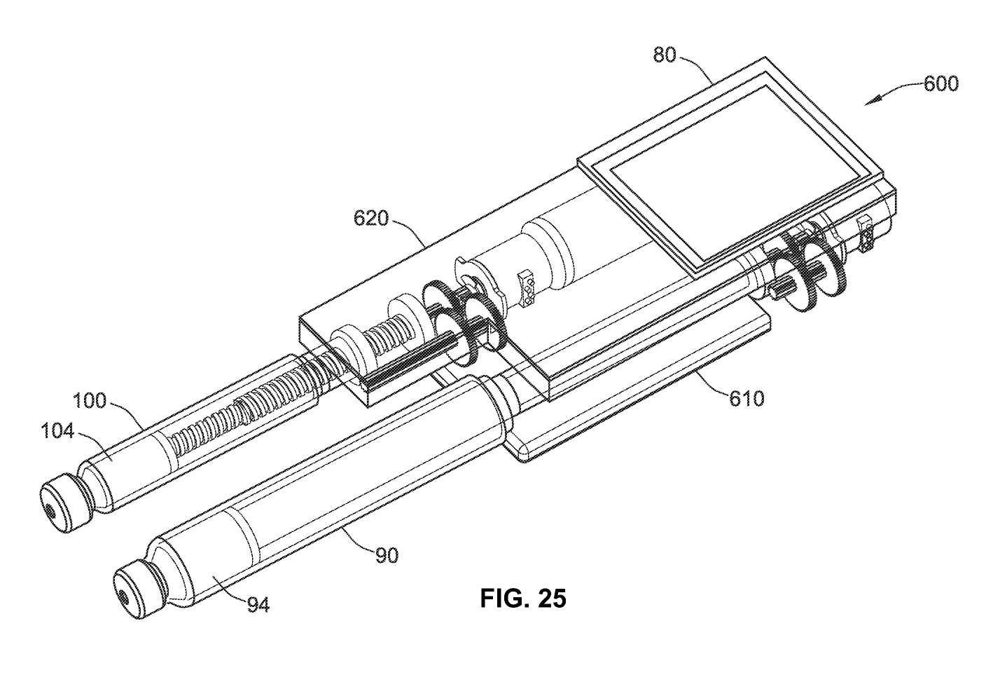

FIG. 24 illustrates various internal components of the drug delivery device 10 illustrated in FIGS. 1a and 1b including a preferred alternative drive train arrangement 600. As illustrated, FIG. 25 illustrates the digital display 80, a printed circuit board assembly (PCBA) 620, along with a power source or battery 610. The PCBA 620 may be positioned between the digital display 80 and a drive train 600 with the battery or power source 610 positioned beneath this drive train. The battery or power source 610 is electronically connected to provide power to the digital display 80, the PCBA 620 and the drive train 600. The digital display 80 and the PCBA 620 of this alternative drive train arrangement 600 operate in a similar manner as previously described.

As illustrated, both the first and second cartridges 90, 100 are shown in an expended state. That is, the first and second cartridges are illustrated in an empty state having a stopper at a most distal position. For example, the first cartridge 90 (which ordinarily contains the first medicament 92) is illustrated as having its stopper 94 at the end or most distal position. The stopper 104 of the second cartridge 100 (ordinarily containing the second medicament) is illustrated in a similar end position.

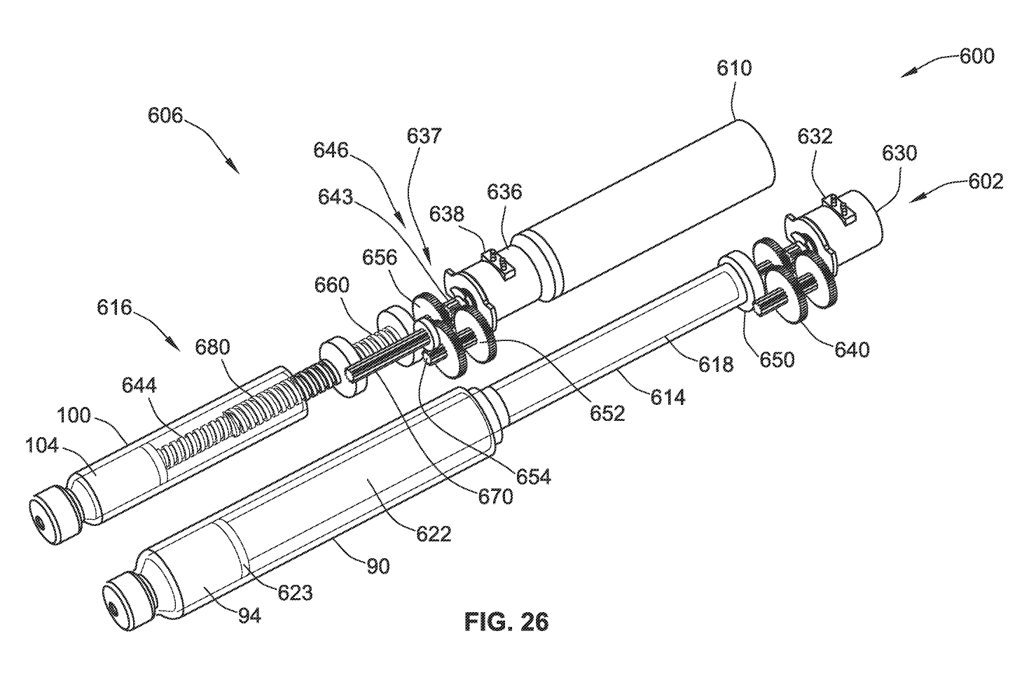

FIG. 26 illustrates the electro-mechanical system illustrated in FIG. 25 with both the digital display 80 and the PCBA 620 omitted. As illustrated, this alternative electro-mechanical system 600 operates to expel a dose from the first cartridge 90 containing a primary medicament 92 and the second cartridge 100 containing a secondary medicament 102. In this preferred electro-mechanical system 600, the system comprises an independent mechanical driver for both the first cartridge and the second cartridge. That is, an independent mechanical driver 602 operates to expel a dose from the first cartridge 90 and an independent mechanical driver 606 operates to expel a dose from the second cartridge 100. If this preferred electro-mechanical system 600 were to be reconfigured to operate on three different medicaments contained within three separate cartridges, three independent mechanical drivers could be provided so as to administer a combined dose. The independent mechanical drivers act under control of the motor drivers 332, 334 of the control unit 300 (see, e.g., FIG. 19).

The first independent mechanical driver 602 operates to expel a dose from the first cartridge 90 and operates in a similar manner as the independent drivers 502, 506 described with reference to the drive train 500 illustrated in FIGS. 21-22 above. That is, this first independent driver 602 comprises a first motor 630 that is operatively coupled to a first gearing arrangement 640. To energize this motor 630, a connector 632 is provided as a means of electrically connecting to the motor driver 332. This first gearing arrangement 640 is mechanically linked to a proximal portion of the telescoping piston rod 614. As this gearing arrangement 640 is driven by an output shaft of the first motor 632, this arrangement 640 rotates the proximal portion 618 of the telescoping piston rod 614. As this proximal portion 618 of the piston rod 614 is rotated, the second or distal portion 622 of the piston rod 614 is driven in a distal direction. Moving in this distal direction, a distal end 623 of the second portion 622 of the piston rod 614 exerts a force on the stopper 94 contained within the first cartridge 90. With a distal end 623 of the piston rod 614 exerting a force on the stopper 94, the user selected dose amount of the first medicament 92 is forced out of the cartridge 90 and into an attached interface hub 200 and consequently out of the dispense interface of a medicated module.

Preferably, the first independent mechanical driver 602 comprises a bung or stopper detection system. Such a detection system may be used detect the position of the cartridge stopper 94 following a cartridge change event. For example, when a cartridge change event occurs, the piston rod is retracted in a proximal position so as to enable a user to open the cartridge retainer and thereby provide access to a spent cartridge. When the cartridge is replaced and the cartridge retainer door is shut, the piston rod will advance in a distal direction towards the stopper of new the cartridge.

In one preferred stopper detection system, a switch is provided at the distal end of the piston rod. Such a switch may comprise a mechanical, optical, capacitive, or inductive type switch. Such a switch would be in communication with the microcontroller and indicates when the piston rod is in contact with the stopper and hence may be used as a mechanism for stopping the drive system.

The second independent mechanical driver 606 operates to expel a dose from the second cartridge 100 in a different manner than the first independent driver 602. That is, this second mechanical driver 606 comprises a second motor 636 that is operatively coupled to a second gearing arrangement 646. To energize this motor 636, a connector 638 is provided as a means of electrically connecting to the motor driver 334.
This independent mechanical driver 606 comprises: a. A motor 636;
b. A second gearing arrangement 646; and
c. A telescope piston rod 616.

The second gearing arrangement 646 is mechanically linked to a proximal portion of a nested piston rod 660. As this gearing arrangement 646 is driven by the output shaft of the second motor 636, this arrangement 646 rotates the proximal portion 660 of the telescoping piston rod 616.

The second gearing arrangement 646 comprises a motor pinion along with a plurality of compound gears (here four compound gears) along with a telescope input piston rod. Two of the compound gears are elongated to enable continuous mesh engagement with the input piston rod as the telescope extends in a distal direction to exert an axially pressure on the cartridge stopper 104 so as to expel a dose from the cartridge. The elongated gear may be referred to as a transfer shaft. The gearbox arrangement preferably has a ratio of 124:1. That is, for every revolution of the telescope input screw the output shaft of the second motor rotates 124 times. In the illustrated second gearing arrangement 646, this gearing arrangement 646 is created by way of five stages. As those skilled in the art will recognize, alternative gearing arrangements may also be used.

The second gearing arrangement 646 comprises three compound reduction gears 652, 654, and 656. These three compound reduction gears may be mounted on two parallel stainless steel pins. The remaining stages may be mounted on molded plastic bearing features. A motor pinion 643 is provided on an output shaft of the second motor 636 and is retained on this shaft 637, preferably by way of an interference or friction fit connection.

As described above, the motor pinion 643 may be provided with two mounted “flag” features that interrupt the motion detect optical sensor. The flags are symmetrically spaced around the cylindrical axis of the pinion.

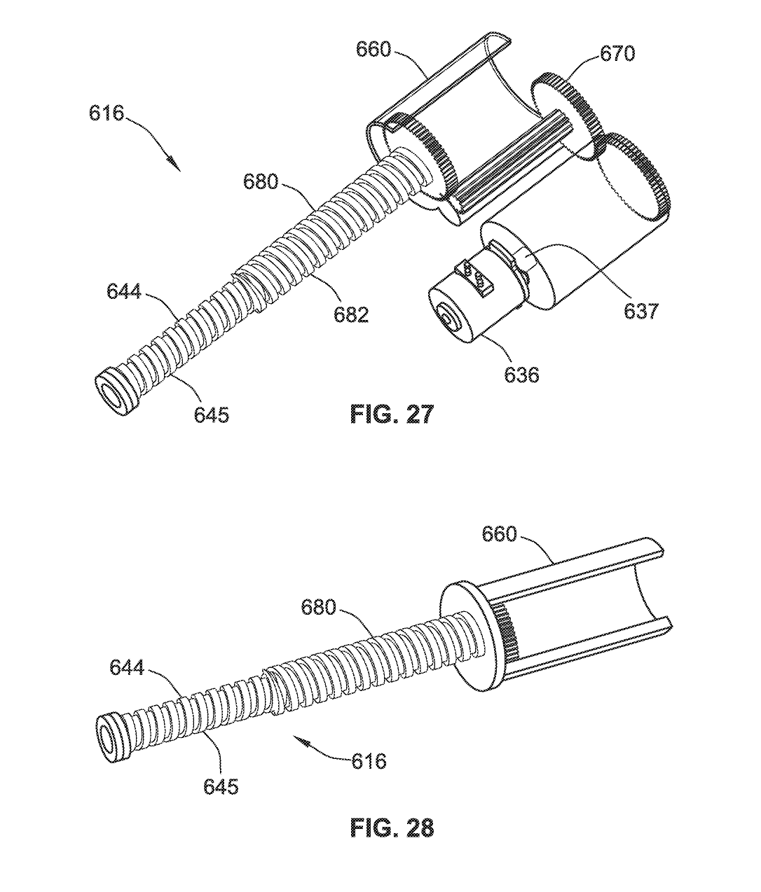

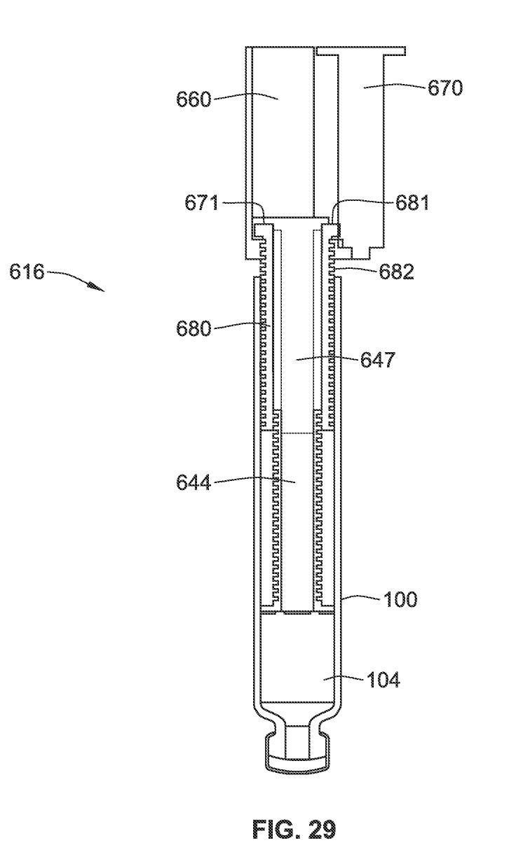

The drive train telescoping piston rod 616 is illustrated in FIG. 27 and comprises a telescope plunger 644 that is operatively coupled to an input screw 680. FIG. 28 illustrates a perspective view of the telescope piston rod 616 coupled to a latch barrel. FIG. 29 illustrates a cross sectional view of the independent mechanical driver with the piston rod 616 in an extended position.

As illustrated, the outer elements (the telescope piston rod plunger 644 and telescope) create the telescopic piston rod 616 and react to the compressive axial forces that are developed. An inner element (telescope piston rod key 647) provides a means of reacting the rotational input force. This operates with a continuous motion and force since there will be no changes in drive sleeve diameter to generate varying levels of force.

The transfer shaft 670 is operatively linked to the gearing arrangement 646. The transfer shaft 670 can rotate but it cannot move in an axial direction. The transfer shaft 670 interfaces with the second gearing arrangement 646 and transfers the torque generated by the second gearbox arrangement 646 to the telescope piston rod 616. Specifically, when the transfer shaft 670 is rotated by way of the gearing arrangement 646, the transfer shaft 670 will act on an integrated geared part 681 on a proximal end of the input screw 680. As such, rotation of the transfer shaft 670 causes the input screw 680 to rotate about its axis.

A proximal portion of the input screw 680 comprises a threaded section 682 and this threaded section is mated with a threaded section of the latch barrel 660. As such, when the input screw 680 rotates, it winds or screws itself in and out of the latch barrel 660. Consequently, as the input screw 680 moves in and out of the latch barrel, the screw 680 is allowed to slide along the transfer shaft 670 so that the transfer shaft and the gears remain mated.

The telescope plunger 644 is provided with a threaded section 645. This threaded section 645 is threaded into short section in distal end of the input screw 680. As the plunger 644 is constrained from rotating, it will wind itself in and out along the input screw 680.

A key 647 is provided to prevent the plunger 644 from rotating. This key 647 may be provided internal to the input screw 680 of the piston rod 616. During an injection step, this key 647 moves in the axial direction towards the stopper 104 of the cartridge 100 but does not rotate. The key 647 is provided with a proximal radial peg that runs in a longitudinal slot in the latch barrel 660. Therefore, the key 647 is not able to rotate. The key may also be provided with a distal radial peg that engage a slot in the plunger 644.

Preferably, the drug delivery device 10 comprises memory devices comprising enough memory storage capability so as to store a plurality of algorithms that are used to define a plurality of different therapeutic profiles. In one preferred arrangement, after a user sets a dose of the primary medicament, the drug delivery device will be preprogrammed so as to determine or calculate a dose of the secondary medicament and perhaps a third medicament based on one of the stored therapeutic profiles. In one arrangement, the healthcare provider or physician selects a therapeutic dose profile and this profile may not be user alterable and/or may be password protected. That is, only a password known by the user, for example a healthcare provider or physician, will be able to select an alternative profile. Alternatively, in one drug delivery device arrangement, the dose profile is user selectable. Essentially, the selection of the therapeutic dose profiles can be dependent upon the individualized targeted therapy of the patient.

As described above, certain known multi drug compound devices allow independent setting of the individual drug compounds. As such, the delivery of the combined dose in a combination is determined by a user. This is not ideal in all the therapeutic situations that a patient may face.

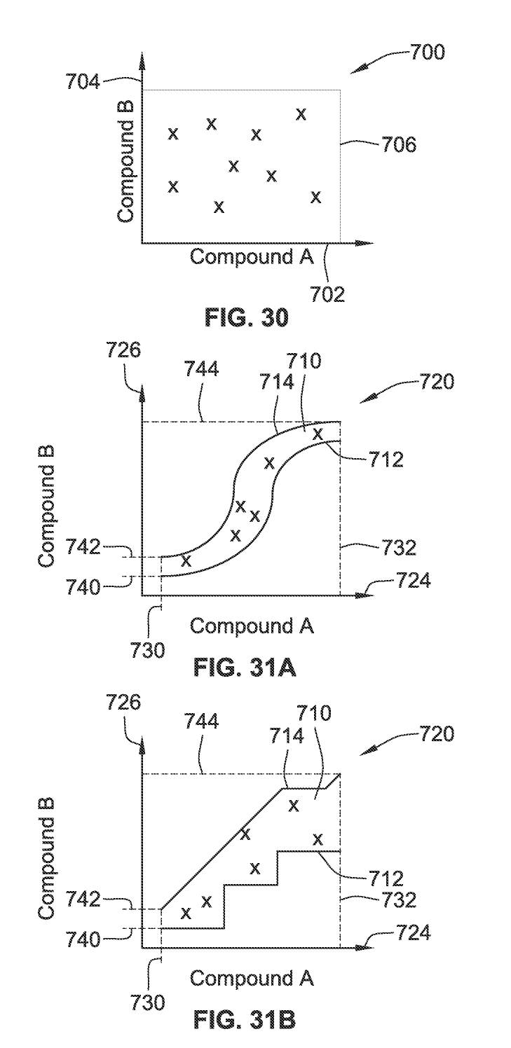

Various therapeutic dose profiles will now be described with reference to FIGS. 30-50. It should be understood that regardless of which dose profile is used with respect to the medicaments contained in the auto-injector device, a fixed dose of the medicament contained in the mediated module will always be delivered therewith.

FIG. 30 illustrates a potential deliverable therapy 700 of such a known two input and two compound combination device: that is, a device that requires a user to physically set the first dose of a first medicament and then physically set the second dose of the second medicament. In such a known device, a user could select a dose of the Compound A or the primary medicament 702 along the x-axis (i.e., between 0 units to a top dose). Similarly, the user could then select a dose of the secondary medicament—Compound B 704 along the y-axis (i.e., between 0 units to a top dose). As such, although these known devices can potentially deliver the combination of the two compounds as illustrated by area 706 shown in FIG. 30, there is an inherent risk that the user does not follow the correct, prescribed therapeutic profile, either intentionally or otherwise. For example, in such a device, the user must know, or be able to determine or calculate, the required relationship and then set the dose of both the first and second compounds 702, 704 independently.

One of the primary reasons for combining drug compounds is that generally all the pharmaceutical elements are required to ensure an increased therapeutic benefit to a patient. In addition, some compounds and some combinations of compounds need to be delivered in a specific relationship with each other in order to provide the optimum pharmacokinetic (“PK”) and pharmacodynamic (“PD”) response. Such complex relationships between one, two, or more medicaments may not be achievable through a single formulation route and could potentially be too complex for the user to understand, or follow correctly, in all cases.

In an example embodiment of the invention, a multi drug compound device may be reliant upon the user input for each independent compound to control the delivered dose profile within predetermined thresholds. For example, FIGS. 31a and 31b illustrate in diagrammatic form a potential delivered therapy 720 of a theoretical two input, two compound combination device. The area 710 illustrates the range of potential combination doses that are achievable. That is, a user can set the dose of the primary medicament or Compound A 724 anywhere from a minimum value 730 to a maximum value 732. Similarly, the user can separately and independently set the dose of the secondary medicament or Compound B 726 anywhere from a minimum value 740 to an overall maximum value 744 within predetermined thresholds, for example between a lower limit 712 and an upper limit 714. In this area 710, the plurality of ‘X’ designations illustrate specific combination doses that a patient and/or user of such a device may elect to set and deliver. Essentially, the combined dose of Compound A 724 and Compound B 726 can be set anywhere within this area 710. In the example embodiment, the user is limited to setting a combined dose only along a predefined profile, such as the predefined profile illustrated by area 710 in FIGS. 31a and 31b. For example, if an amount of Compound A is selected by a user to be the minimum value 730, Compound B may be selected between the minimum value 740 and a maximum value 742 defined for this minimum value of Compound A.

The lower limit 712 and the upper limit 714 may be represented by a curve as in FIG. 31a. In an alternative embodiment, the lower limit and the upper limit may be represented by one or more lines, by a stepwise function, and/or the like. For example, in the diagram of FIG. 31b, the upper limit 714 is represented by a diagonal line and a horizontal line, the lower limit 712 is represented by a stepwise function of 3 steps. The upper limit 714 and the lower limit 712 define an area 710, in which a user may select a combination of Compound A and Compound B, for example one of the combinations designated by the ‘X’-marks.

In further example embodiments, the presently proposed programmable electro-mechanical auto-injector drug delivery device described in detail above uses only a single input in order to offer an innovative solution to these and other related problems. Further, the proposed programmable multi-drug compound device uses only a single dispense interface (i.e., the dispense interface of the medicated module). As just one example, such a device is capable of delivering any of a plurality of predefined programmed therapeutic profiles for various drug combinations. As an alternative, such a device is capable of delivering only one predefined programmed therapeutic profile for various drug combinations.

By defining the ratio-metric relationship or relationships between the various individual drug compounds (2, 3, or more), the proposed device helps to ensure that a patient and/or user receives the optimum therapeutic combination dose from a multi drug compound device. This can be accomplished without the inherent risks associated with multiple inputs. This can be achieved since the patient and/or user is no longer called upon to set a first dose of medicament and then determine or calculate and then independently set a correct dose of a second and/or third medicament in order to arrive at the correct dose combination each time the device is used to administer a combination dose.

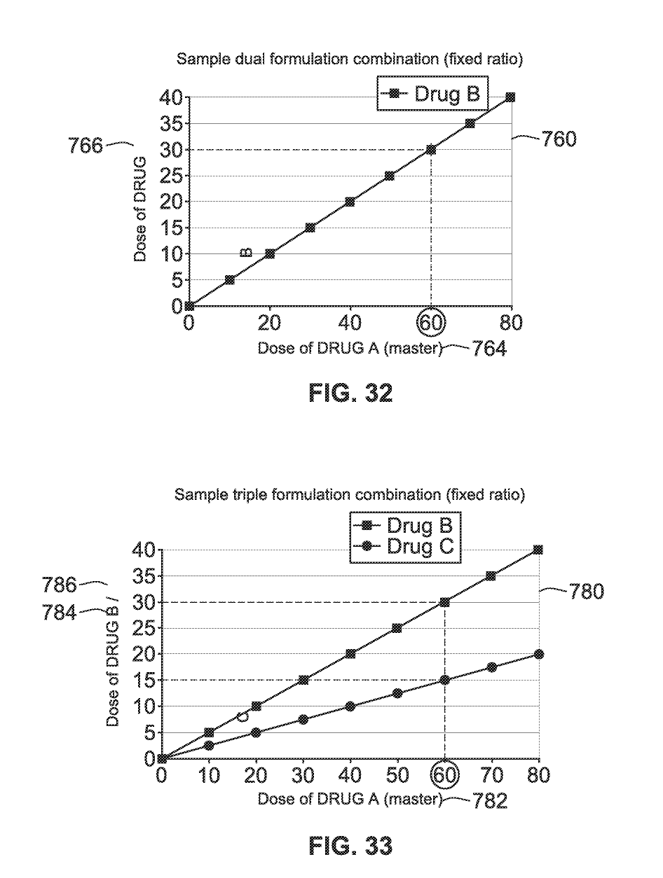

As just one example, FIG. 32 illustrates a first arrangement of a predefined therapeutic profile 760 that may be programmed into Applicants' programmable drug delivery device. In FIG. 32, a first therapeutic dose line represents an example of a predefined therapeutic profile 760 compared to the area 706 indicating all potential drug combinations that can be selected by way of currently known devices as illustrated in FIG. 30. As can be seen from this predefined profile 760 illustrated in FIG. 32, for every dose value of Compound A 764 (also herein referred to as the Master Drug or the Primary Drug or the Primary Medicament) selected by the user, Applicants' drug delivery device 10 will rely on a previously stored therapeutic profile to calculate the dose value of Compound B 766 along this therapeutic profile 760.

As such, the user merely needs to select a first dose of the first drug: Drug A or the primary medicament and Applicants' drug delivery device 10 automatically calculates the dose of the secondary medicament or Drug B based on this preselected dosing profile 760. For example, if the user selects a dose comprising “60 Units” for Compound A 764, the drug delivery device 10 will recall the selected dosing profile 760 from its memory device and then automatically calculate the dose value of “30 Units” for Compound B 766.

In an alternative drug delivery device arrangement, and as discussed in greater detail above, the drug delivery device may comprise a coding system. A coding system may be provided if coding means is provided on either the first or the second cartridge so that the drug delivery device could then identify the particular medicament contained within an inserted cartridge. After the drug delivery device undergoes a method or process for determining cartridge and/or medicament identification, the drug delivery device could then potentially automatically update the therapeutic profile or profiles. For example, a new or a revised/updated profile may be selected if required to reflect an updated or revised pharmaceutical philosophy so as to achieve an optimum medicament relationship. Alternatively, a new or a revised/updated profile may be selected if a health care provider has decided to alter a patient's therapy strategy. An updated or revised profile may be loaded into the device through a wired or wireless connection, for example from a memory comprised in the cartridge, from an external device, from the internet and/or the like. The updated or revised profile may be loaded automatically, for example after insertion of the cartridge, or only after user confirmation, for example after a user presses a button on the device to confirm a message shown in the display.

As another example of a therapeutic profile, the proposed drug delivery device 10 may be programmed to calculate a linear ratio profile for the delivered dose from the drug delivery device 10 that comprises two or more discrete medicament reservoirs. For example, with such a programmed therapeutic profile, the constituent components of the dose would be delivered to a patient in a fixed, linear ratio. That is, increasing the dose of one element will increase the dose of the other constituent element(s) by an equal percentage. Similarly, reducing the dose of one element will reduce the dose of the other constituent element(s) by an equal percentage.

FIG. 32 illustrates one arrangement of a predefined ratio therapeutic profile 760 that may be programmed into the drug delivery device 10. In the profile illustrated in FIG. 32, the user would select a dose of Drug A 764. As previously described above, the user could be called upon to select this first dose by toggling or manipulating one of the buttons provided on the operator interface of the drug delivery device 10. Once this initial dose of the primary Drug A 764 is selected by the user and then set by the drug delivery device, the control unit of the device 10 calculates and then sets the resultant dose of Drug B 766 based on the therapeutic profile 760. For example, referring to FIG. 32, if the user selects a dose of 60 units for Drug A 764, the control unit would recall the algorithm for this particular therapeutic profile 760 and would then use this algorithm to calculate the dose of Drug B or the secondary medicament 766. According to this profile 760, the control unit would calculate a 30-Unit dose of Drug B or the secondary medicament. In an alternative embodiment, the profile is stored as a look-up table in a memory. For every value of drug A, a corresponding value of drug B is stored in the look-up table. In a further embodiment only some values of drug A are stored in the look-up table along with corresponding values of drug B. Missing values are then calculated by interpolation, for example by linear interpolation.

Therefore, when the device is then used to dispense the combination of medicaments, this combined dose comprising 60 Units of Drug A and 30 Units of Drug B would be administered. As those of skill in the art will recognize, the ratio of the two (or more) medications can be tailored according to the needs of the patient or therapy by a number of methods including changing the concentration of the medicaments contained within the primary or secondary reservoirs.

In one example, the auto-injector device 10 may comprise three or more medicaments. For example, the device 10 may contain a first cartridge containing a long acting insulin, a second cartridge containing a short acting insulin, and a third cartridge containing a GLP-1. In such an arrangement, referring back to FIGS. 6 and 9, the cartridge holder 40 of the drug delivery device 10 would be re-configured with three cartridge retainers (rather than the two retainers 50, 52 illustrated in FIGS. 6 and 9) and these three cartridge retainers would be used to house three compound or medicament cartridges. FIG. 33 illustrates an arrangement of a predefined fixed ratio therapeutic profile 780 that may be programmed into the proposed drug delivery device 10. FIG. 33 illustrates a linear dose profile 780 that may be used with a drug delivery device comprising three medicaments. For example, in this profile, the user would first select a dose of 60 Units of the primary medicament—Drug A 782. Once this initial dose of Drug A 782 has been selected, the control unit of the device 10 would calculate, based on this selected therapeutic profile 780, the resultant dose amount of Drug B (the secondary medicament) 784 as well as the resultant dose of Drug C (the tertiary medicament) 786. When the device 10 is then used to dispense the combined dose of medicaments, the combination dose of 105 Units would comprise a combination dose of 60 Units of Drug A, a calculated dose of 30 Units of Drug B 784, and a calculated dose 15 Units of Drug C 786. In such an arrangement, the primary or master drug 782 could comprise an insulin or insulin analog, the secondary medicament 784 could comprise a GLP-1 or GLP-1 analog, and the tertiary medicament 786 could comprise a local anesthetic or anti-inflammatory.

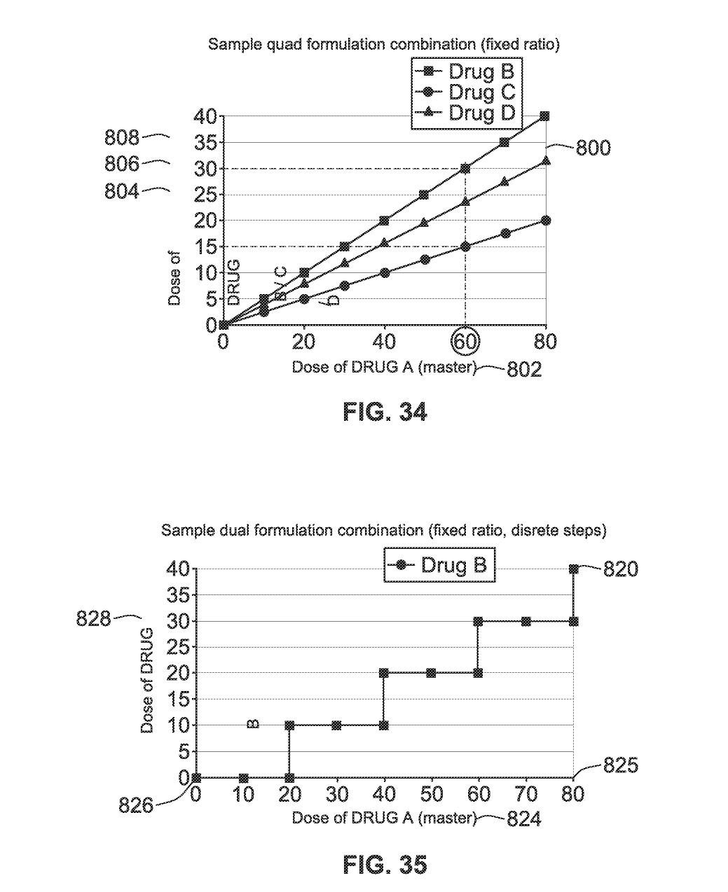

Similarly, FIG. 34 illustrates an alternative arrangement of a predefined fixed ratio therapeutic profile 800 that may be programmed into the drug delivery device 10 illustrated in FIG. 1. FIG. 34 illustrates a linear profile for use with a drug delivery device comprising four different medicaments: Drug A 802, Drug B 804, Drug C 806, and Drug D 808. Again, in this situation, once the initial dose of the primary medicament (i.e., Drug A) 802 has been selected by the user, the control unit of the device 10 calculates, based on this linear profile 800, the resultant dose amount of Drug B 804, Drug C 806, and Drug D 808. For example, in this illustrated exemplary profile, a user has selected a 60 Unit dose of Drug A or the primary medicament 802. With such a selected primary dose, when the device 10 is then used to dispense the calculated combined dose, the combination dose of 129 Units would comprise 60 Units of the selected Drug A 802, 30 Units of Drug B 804, 24 Units of Drug D 806, and 15 Units of Drug C 808.

A derivative therapeutic profile of the various profiles illustrated in FIGS. 32-34 may be provided for the combination of compounds to be delivered in a fixed ratio, but for the dose setting process for the master drug compound (i.e., Drug A) to only allow doses of the secondary compound or medicament to be calculated in discrete amounts. This would mean that the dose of the dependent drug compound or compounds (e.g., Drug B, Drug C, etc.) or the secondary medicaments would also only be calculated in discrete amounts.

For example, FIG. 35 illustrates an alternative arrangement of a predefined fixed ratio therapeutic profile 820 having discrete dose steps and that may be programmed into the drug delivery device 10. For example, this profile 820 comprises a fixed ratio profile having five (5) discrete dose steps of Drug B 828 for varying amounts of Drug A 824.

While following the fixed ratio profile, Drug A 824 would be continuously variable between a maximum dose 825 and a minimum dose 826 while the calculated dose of the secondary medicament 828 would not be continuously variable. For example, if a user were to select a dose of either 0 or 20 Units of the master medicament Drug A 824, the drug delivery device 10 would determine a zero (“0”) dose of Drug B 828. Similarly, if a user were to select a dose of anywhere from 20-40 Units of the Drug A 824, the drug delivery device 10 would compute a dose of 10 Units of Drug B 828. Therefore, in this later case, a combination dose of 20 Units of Drug A 824 would result in a maximum dose of 10 Units of Drug B 828.

Applicants' proposed linear ratio profile discussed and described with respect to FIGS. 32-34 provides a number of advantages. For example, these various proposed linear ratio profiles are analogous to a profile of a single formulation product that contains a combination of two or more therapeutic medicaments, where the concentration of the formulation is constant. This means that with the proposed drug device 10 programmed with such linear ratio profiles 760, 780, 800 and 820, this would provide an alternative delivery platform for scenarios where it is not possible to formulate the individual elements together into a single formulation. This may be the case where mixing such medicaments may raise stability, compromised performance, toxicology issues and/or other related types of issues.

In addition, the proposed linear ratio therapy profiles 760, 780, 800 and 820 are robust to a split dosing requirement. That is, the desired dose can potentially be split into multiple, smaller injections without compromising the total amount of each constituent medicament that is ultimately administered. As just one example, returning to FIG. 32, if the patient were to split up a 60 Unit dose into a 30 Unit dose followed by two 15 Unit doses, the net result (in terms of the total amount of each of the constituent elements delivered) would be the same. Such a split dosing requirement might be advantageous in situations where the calculated combined dose is a large dose (e.g., where the injected dose is greater than 1 ml), where the delivery of such volumes to a single injection site might be painful for a particular patient or sub-optimal in terms of its absorption profile.

In addition, cognitively, the relationship between the various compounds or drugs is reasonably straightforward for a patient to understand. Moreover, with such profiles 760, 780, 800 and 820, the patient and/or health care provider is not called upon to perform profile calculations themselves since it is the microcontroller of the device 10 that computes the value of the secondary medicament automatically once the initial dose of the primary medicament has been set.

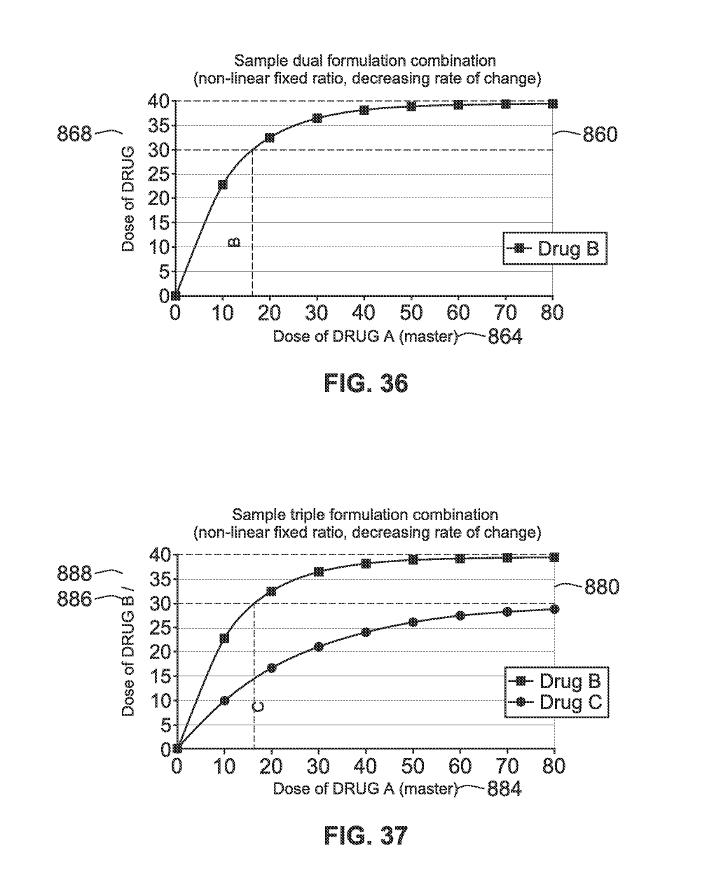

FIG. 36 illustrates another proposed therapy profile 860 that might be programmed into the control unit of the drug delivery device 10. This profile 860 comprises a non-linear ratio dose profile. With such a programmed profile, the constituent components of the dose would be delivered to a patient in a fixed, non-linear ratio. That is, the relationship between the size of the delivered dose of the primary medicament and that of the secondary medicament and perhaps a third medicament is fixed, but is non-linear in nature. With such profiles, the relationship between the primary and the secondary medicament might be cubic, quadratic, or other similar type of relationship.

As described above, the delivery of a combination of drug products (i.e., single doses that are made up from the combination of two or more individual drug formulations) in a format where the ratio-metric profile is predefined, offers a number of benefits for both a patient and the treatment of a particular condition. For certain combinations, the ideal profile might be for the various individual formulations to be delivered in a defined, non-linear ratio to one another. Therapeutic profiles of this type are not achievable from a combination drug or drugs that is co-formulated into a single drug reservoir, such as, but not limited to, a standard 3 ml glass cartridge. In such situations, the concentration of the various constituent parts within the glass cartridge is constant (i.e., xmg/ml), and would be particularly difficult for a patient to calculate on certain known devices for each dose. To calculate or determine such concentration would be reliant on the patient or health care provider being able to look up the correct dose on a table (or similar lookup document or prescription) and this may be less desirable as such a method would be more prone to error.

FIGS. 36-39 illustrate exemplary profiles 860, 880, 900 and 920 utilizing non-linear dose profiles. For example, FIG. 36 illustrates an arrangement of a predefined non-linear fixed ratio therapeutic profile 860 having a decreasing rate of change. That is, as the amount of the primary medicament Drug A 864 increases, the amount of the secondary medicament Drug B 868 increases sharply, as, for example, the amount of Drug A increases from 0 Units to approximately 30 Units and quickly tapers off thereafter. As such, FIG. 36 illustrates a sample dual formulation wherein the profile 860 is non-linear.

FIG. 37 illustrates a similar profile 880 but a profile that represents a sample triple formulation combination of three different medicaments: Drug A 884, Drug B 886 and Drug C 888. As just one example, with this profile 880, if the user sets a dose of 50 Units of the master Drug A 884, the control unit of the device 10 will compute a resulting combined dose comprising approximately a 37 Unit dose of Drug B 886 and an approximately 26 Unit dose of Drug C 888.

Some of the advantages of using such a fixed, non-linear ratio of the constituent drug elements as illustrated include (but are not limited to) the fact that such profiles utilize a decreasing rate of change profile. These types of illustrated therapy profiles 860, 880 may be appropriate in situations where it is desirable to initially rapidly increase the dose of Compound B or the secondary medicament, relative to Compound A. However, once the desirable dose range has been reached to slow this rate of increase so that the dose does not then increase much further, even if the dose of Compound A doubles, for example. A profile of this type might be beneficial in therapeutic applications where there are a potentially wide range of doses of Compound A that patients might require (either as an individual, or across the therapy area as a whole), but where there is a much narrower therapeutically beneficial range of doses for Compound B.

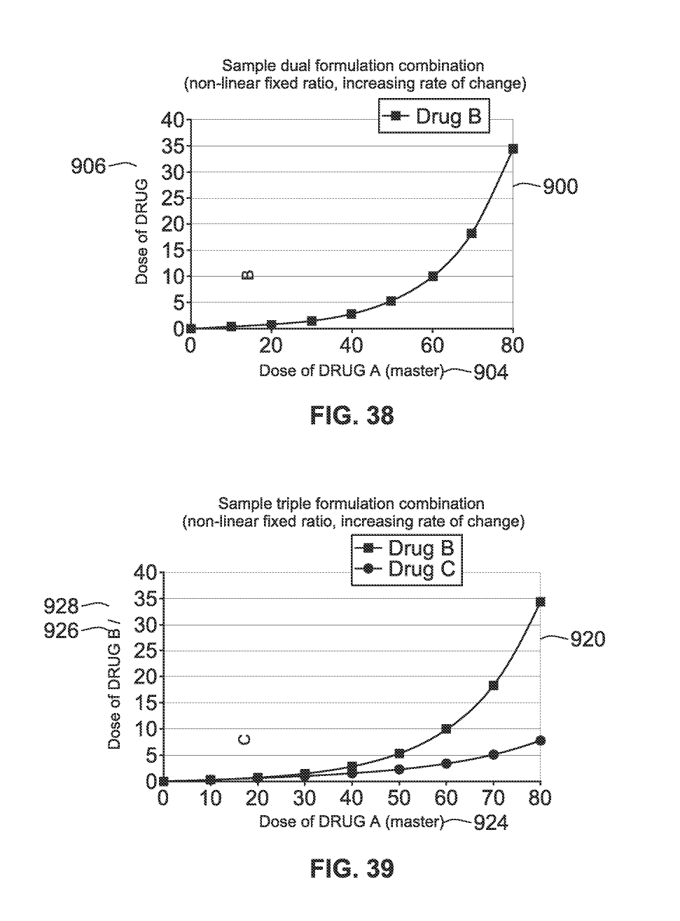

The dose profiles 860, 880 illustrated in FIGS. 36 and 37 provide a non-linear fixed ratio having a decreasing rate of change. Alternatively, a proposed non-linear fixed ratio dose profile may comprise a profile having an increasing rate of change. For example, one such profile 900 having such a non-linear increasing rate of change within a two medicament drug delivery device such as device 10 is illustrated in FIG. 38. FIG. 39 illustrates a non-linear fixed ratio profile 920 having such an increasing rate of change within a three medicament drug delivery device. With this profile 920, as the size of the user selected dose of Drug A 924, the incremental increase in the computed dose of Drug B 926 and Drug C 928 increases.

Applicants' therapeutic profiles 900 and 920 illustrated in FIGS. 38 and 39 might be advantageous in situations where a patient receiving a low dose of Compound A (e.g., 0-40 Units of Drug A 904) may only require a relatively low dose of Compound B 906 for the desired pharmokenitic therapeutic response. However, as the size of the dose of Compound A 904 increases, the dose of Compound B 906 needs to provide the same therapeutic response increase at a much greater rate.

Alternatively, the drug delivery device 10 may be programmed with an algorithm for computing a dose of the secondary medicament based on a fixed, linear ratio followed by a fixed dose profile. As just one example, such a stored profile may initially follow a fixed ratio profile for certain low doses of the primary medicament or Compound A. Then, above a certain threshold dose level of the Drug A, the profile switches to a fixed dose of the secondary medicament or Compound B. That is, for higher doses of the primary medicament/Compound A, the secondary medicament will comprise essentially a fixed dose.

For certain therapies, the delivery of combination drug products (i.e., single doses that are made up from the combination of two or more individual drug formulations) might be beneficial for the dose of the secondary medicament to initially rise rapidly relative to the primary medicament. Then, once a pre-determined threshold value of the primary medicament has been reached, the profile will then flatten out. That is, the calculated dose of the secondary medicament will remain constant regardless of further increases in the set dose of the primary medicament. Such fixed ratio followed by fixed dose-low dose threshold therapeutic profiles are not achievable from a combination drug that is co-formulated into a single primary pack (such as, but not limited to, a standard 3 ml glass cartridge) where the concentration of the various constituent parts is constant (xmg/ml). Achieving such profiles would also be particularly difficult for a patient to calculate on current devices for every dose.

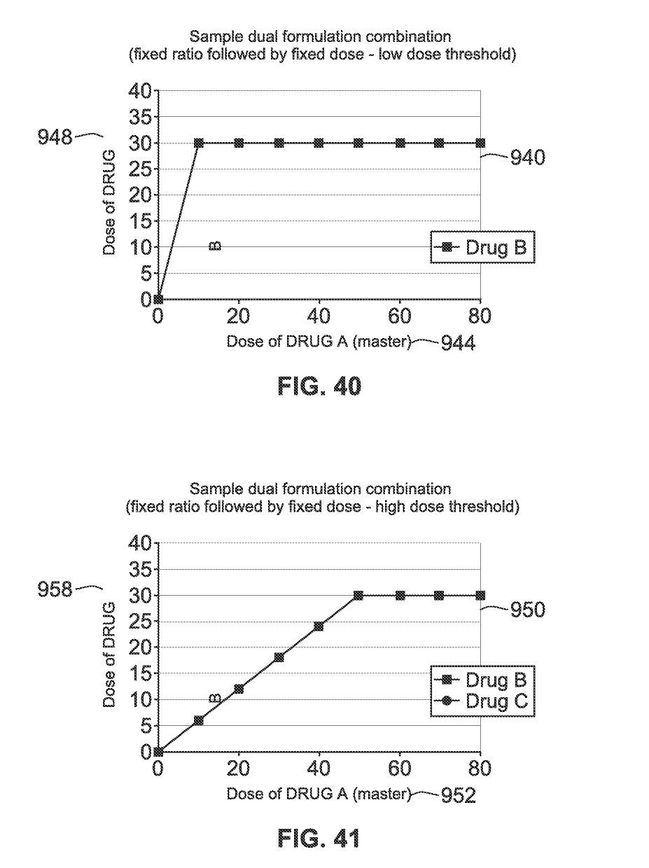

FIGS. 40-42 provide three illustrative examples of such fixed ratio followed by fixed dose-low dose threshold therapeutic profiles 940, 950, and 960. For example, FIG. 40 illustrates an arrangement of a predefined fixed ratio-fixed dose therapeutic profile 940 having a low dose threshold and that may be programmed into the drug delivery device. As illustrated, this profile 940 initially follows a fixed ratio profile for a 0-10 Unit selected doses of the primary medicament or Compound A 944. Then, once this 10 Unit threshold dose level of the Drug A has been surpassed, the profile 940 switches to a 30 Unit fixed dose of the secondary medicament or Compound B 948. As such, for doses greater than 10 Units of the primary medicament/Compound A 944, the secondary medicament 948 will comprise a fixed dose at 30 Units.

FIG. 41 illustrates an alternative arrangement of a predefined fixed ratio-fixed dose therapeutic profile 950 having a high dose threshold. As illustrated, this profile 950 initially follows a fixed ratio profile for a 0-50 Unit selected dose of the primary medicament or Compound A 952. Then, above this 50 Unit threshold dose level of the Drug A 952, the profile 950 switches to a 30 Unit fixed dose of the secondary medicament or Compound B 958. As such, for doses greater than 50 Units of the primary medicament/Compound A 952, the secondary medicament 958 will comprise essentially a fixed dose at 30 Units.

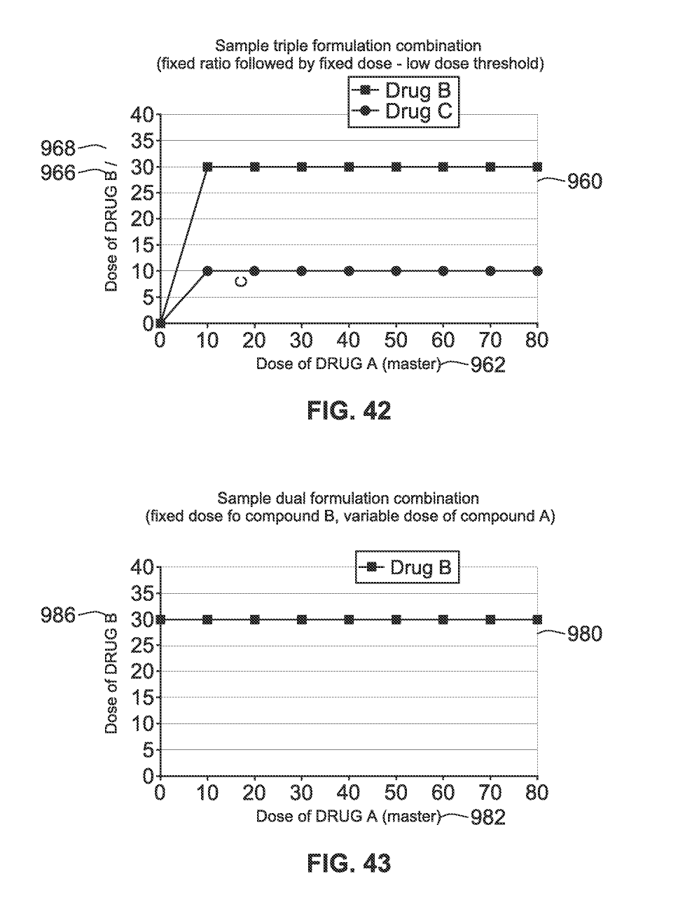

FIG. 42 illustrates an alternative arrangement of a predefined fixed ratio-fixed dose therapeutic profile having a low dose threshold and that may be programmed into the drug delivery device comprising three compounds or medicaments. As illustrated, this profile 960 initially follows a fixed ratio profile for both Drug B 966 and Drug C 968 for a 0-10 Unit selected dose of the primary medicament or Compound A 944. Then, above this 10 Unit threshold dose level of the Drug A, the profile 960 switches to a 30 Unit fixed dose of the secondary medicament or Compound B 966 and a 10 Unit fixed dose of the tertiary medicament Compound C 968. As such, for doses greater than 10 Units of the primary medicament/Compound A 944, the secondary and tertiary medicaments 966, 968 will comprise essentially fixed doses at 30 Units and 10 Units, respectively.

Profiles 940, 950, and 960 deliver a fixed ratio up to a first point and thereafter deliver a fixed dose type of profile thus providing a number of advantages. For example, where priming of the drug delivery device may be required (either for initial first time use, or prior to each dose), these types of a predefined fixed ratio-fixed dose therapeutic profiles facilitate priming of both compounds with potentially minimal wastage. In this regard, these profiles have certain advantages over other programmable therapeutic profiles, such as the fixed dose profiles and the delayed fixed dose profiles described herein below. This may be especially true with regards to wastage of the secondary medicament or Compound B.

In addition, the various profiles described and illustrated in FIGS. 40-42 may be appropriate in treatment situations where it is desirable to rapidly increase the dose of the secondary medicament, relative to the primary medicament initially. However, once a preset dose threshold has been reached, the secondary medicament may be kept constant regardless of further increases in the dose of the primary medicament. As such, this type of profile might be beneficial for drug delivery devices where an initial titration phase (of both drug compounds) is either required, or is deemed preferable for a patient.

An example of a particular combination therapy where profiles 940, 950 and 960 might be appropriate is for the combined delivery of a long acting insulin or insulin analog (i.e., Drug A or the primary medicament) in combination with an active agent, such as a GLP-1 or GLP-1 analog (i.e., Drug B or the secondary medicament). In this particular combination therapy, there is a reasonable variation in the size of the insulin dose across patient population, whereas the therapeutic dose of the GLP-1 may be considered as broadly constant (except during the titration phase) across the patient population.

Another preferred dose profile for use with the drug delivery device 10 comprises a fixed dose of the secondary medicament (i.e., Compound B) and a variable dose of the primary medicament (i.e., Compound A) profile. With such a therapeutic profile, the profile describes the delivery of a fixed dose of Compound B across the full range of potential doses of Compound A.

This fixed dose-variable dose therapeutic profile may be beneficial for the dose of Compound B to be constant for all potential doses of Compound A. One advantage of having the control unit programmed with such a profile is that fixed dose-variable dose therapeutic profiles are not achievable from a combination drug that is co-formulated into a single primary pack (such as, but not limited to, a standard 3 ml glass cartridge) where the concentration of the various constituent parts is constant (xmg/ml).

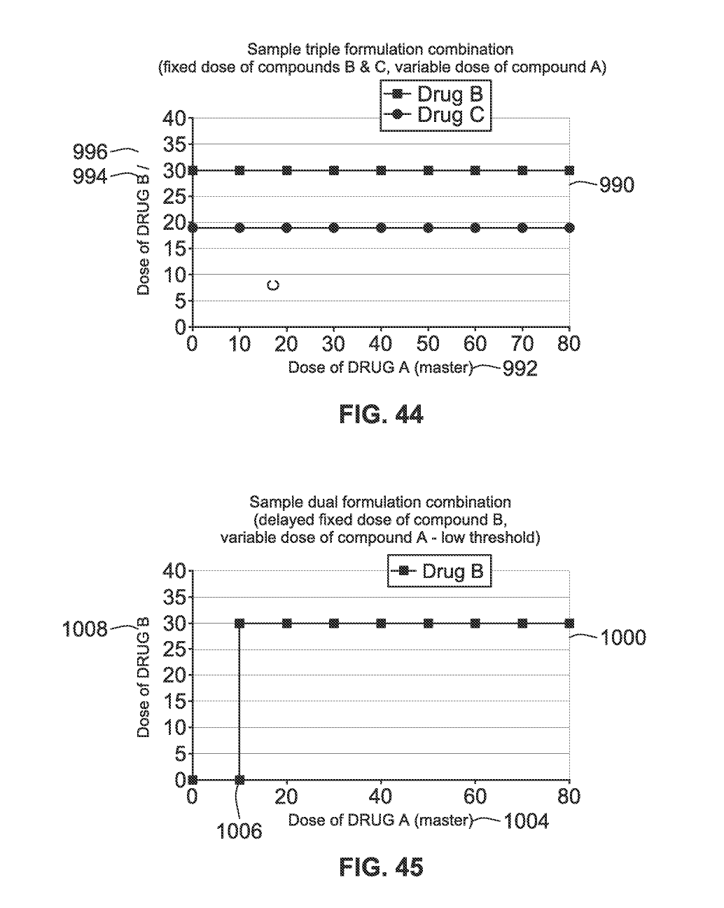

Two such fixed dose-variable dose profiles are illustrated in FIGS. 43-44. FIG. 43 illustrates an arrangement of a predefined fixed dose-variable dose therapeutic profile 980 that may be programmed into the drug delivery device. More specifically, FIG. 43 illustrates a sample formulation combination for a fixed dose of Compound B 986 and a variable dose of compound A 982. As illustrated, for any selected dose of the primary medicament 982, a fixed dose of 30 Units of Drug B 986 will be computed.

FIG. 44 illustrates an alternative arrangement of a predefined fixed dose-variable dose therapeutic profile 990 that may be programmed into the drug delivery device. As illustrated, profile 990 provides for a sample triple formulation combination of a fixed dose of Drug B 994 and Drug C 996 and a variable dose of Drug A 992. As illustrated, for any selected dose of the primary medicament 992, a fixed dose of 30 Units of Drug B 994 and a fixed dose of 18 Units of Drug C 996 will be computed by the drug delivery device 10.

Such fixed dose-variable dose profiles 980 and 990 offer a number of advantages. For example, one of the benefits of these types of delivery profiles is in treatment situations where it is therapeutically desirable to ensure that patients receive a specific dose of one drug compound, irrespective of the size of the variable dose selected of the other compound. This particular profile has specific advantages over other predefined profiles (e.g., the fixed ratio then fixed dose profiles described above, the delayed fixed dose of compound B, variable dose of compound A profiles described below and the controlled thresholds profiles described below), there is not a predetermined minimum dose threshold of primary medicament required to ensure a complete dose of the secondary medicament.

One example of a particular combination therapy where this type of fixed dose-variable dose profile might be particularly appropriate is for the combined delivery of a long acting insulin (i.e., the variable dose) with a GLP-1 (i.e., the fixed dose). In this particular combination, there is reasonable variation in the size of the insulin dose across the patient population, whereas the GLP-1 dose is broadly constant (except during the titration phase where it generally increases in stepped intervals) across the patient population. For this particular therapy regimen, titration of the GLP-1 dose may be needed during the early stages of treatment. This could be achieved with a combination device using different ‘strengths’ of drug within the GLP-1 primary pack (e.g., using 10, 15 or 20 g per 0.1 ml concentrations).

For certain therapies it might be beneficial for the dose of secondary medicament Compound B to be a constant dose once a minimum threshold dose of the primary medicament Compound A has been met and/or exceeded. Again, such profiles of this type are not achievable from a combination drug that is co-formulated into a single reservoir or cartridge (such as, but not limited to, a standard 3 ml glass cartridge). In such standard cartridges, the concentration of the various constituent parts is constant (xmg/ml).

In one arrangement, Applicants' drug delivery device 10 may also be programmed with a therapeutic profile that calculates a delayed fixed dose of a secondary medicament Compound B and variable dose of a primary medicament Compound A. Such a profile provides for the delivery of a fixed dose of Compound B but provides this fixed dose only after a minimum threshold dose of Compound A has been met or exceeded.

Illustrative examples of four predefined delayed fixed dose-variable dose therapeutic profiles 1000, 1020, 1040 and 1060 are illustrated in Applicants' FIGS. 45-48. For example, FIG. 45 illustrates an arrangement of a predefined delayed fixed dose-variable dose therapeutic profile 1000 having a low threshold. More specifically, FIG. 45 illustrates a sample dual formulation combination having a delayed fixed dose of the secondary medicament (i.e., Compound B) and a variable dose of the primary medicament (i.e., Compound A) with the primary medicament having a low dose threshold 1006.

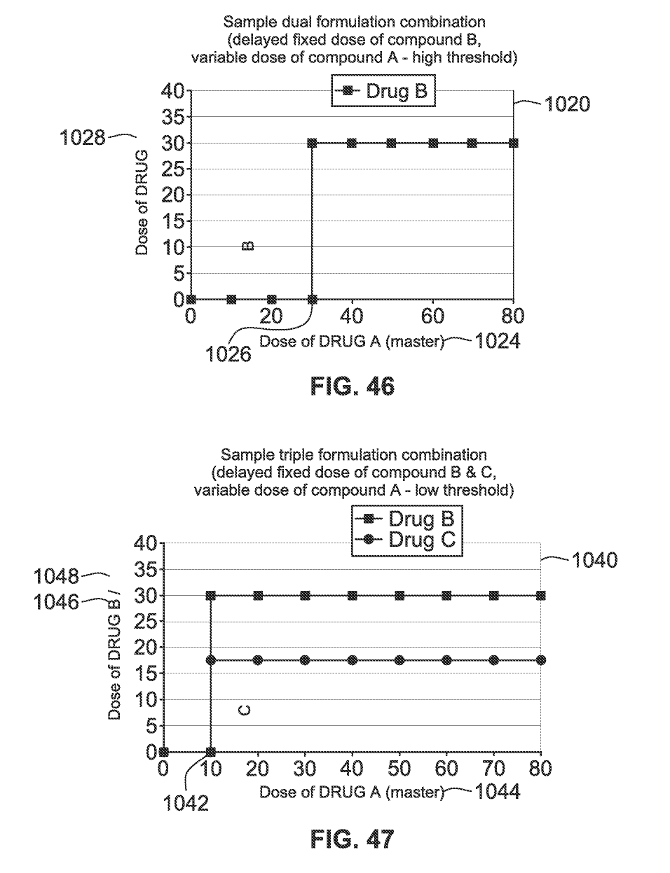

As illustrated in FIG. 45, the profile 1000 defines a variable dose of Drug A 1004 from a minimum dose of 0 Units to a maximum dose of 80 Units. In this illustrative profile 1000, the low threshold 1006 for Drug A 1004 is 10 Units. Based on profile 1000, if a user were to select a dose of Drug A 1004 anywhere from 0 to 10 Units, the control unit would calculate a dose of Drug B 1008 equal to “0” Units. Only after a minimum or threshold dose of 10 units were selected for the primary medicament 1004, would a dose of Drug B 1008 be calculated above “0” Units. Moreover, this calculated dose of Drug B 1008 would be a constant 30 Units, irrespective of the amount of the selected dose set of Drug A 1004, as long as this selected dose remains greater than 10 Units. FIG. 46 illustrates an arrangement of a predefined delayed fixed dose-variable dose therapeutic profile 1020 having a high threshold of Drug A 1024. More specifically, FIG. 46 illustrates a profile 1020 for defining a dual formulation combination having a delayed fixed dose of Compound B 1028 and a variable dose of Compound A 1024. In this illustrative profile 1020, the high threshold 1026 for Drug A 1024 is 30 Units. This high initial threshold 1026 of Drug A 1024 is required before the profile 1020 allows a dose to be set from Drug B 1028. In this illustrated profile 1020, this high initial threshold 1026 equal to 30 Units of Drug A 1024 must be surpassed before the Applicant's delivery device 10 begins to calculate a 30 Unit dose of Drug B 1028.

FIG. 47 illustrates an alternative arrangement of a predefined delayed fixed dose-variable dose therapeutic profile 1040 wherein the drug delivery device 10 comprises two compounds or medicaments. More particularly, FIG. 47 illustrates a profile 1040 for defining a sample triple formulation combination having a delayed fixed dose of Drug B 1046 and Drug C 1048, a variable dose of Drug A 1044 wherein this Drug A 1044 has a low threshold. In this illustrated profile 1040, Drug A 1044 has a low threshold 1042 equal to 10 Units. That is, once a user equals or surpasses the low threshold 1042 of 10 Units of Drug A 1044, the drug delivery device 10 will calculate a dose of 17.5 Units of Drug C 1048 and calculate a dose of 30 Units of Drug B 1046.

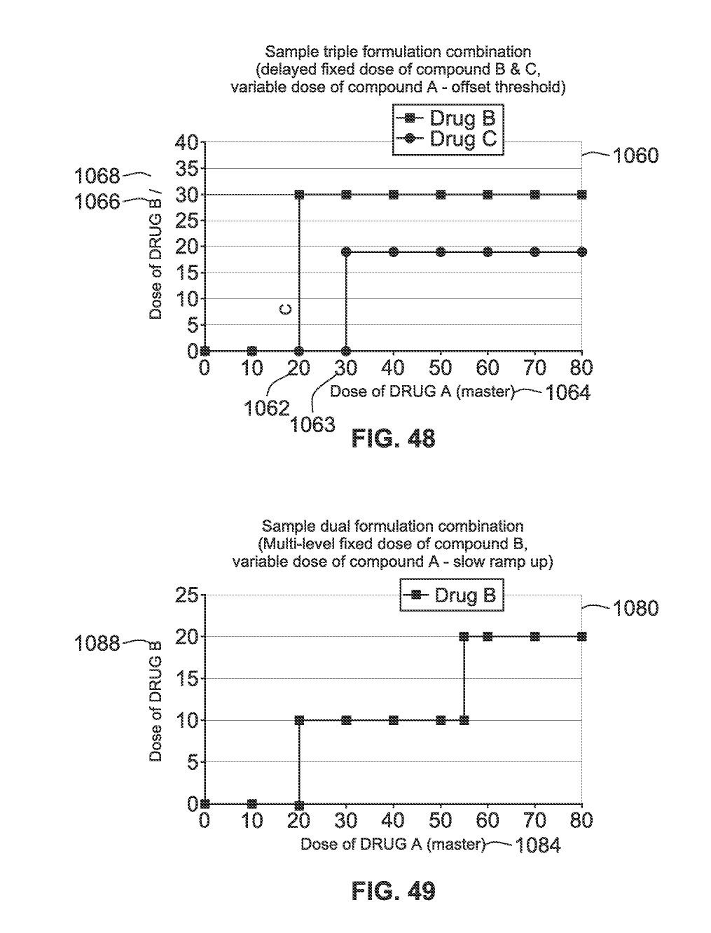

FIG. 48 illustrates a profile 1060 that defines a sample triple formulation combination having a delayed fixed dose of Drug B 1066 and Drug C 1068, and a variable dose of Drug A 1064. In profile 1060, the primary medicament Drug A has two offset thresholds 1062, 1063. That is, once the user selects a dose that surpasses the low threshold 1062 of 20 Units of Drug A 1064, the drug delivery device 10 will calculate a dose of 30 Units for Drug B 1066 and will calculate a dose of “0” Units for Drug C 1068.

Similarly, if a user selects a dose of Drug A 1064 between 20 Units and 30 Units, again the drug delivery device 10 will calculate a dose of 30 Units for Drug B 1066 and calculate a dose of “0” Units for Drug C 1068. Then, it is only after a user selects a dose greater than 30 Units for Drug A 1064 thereby surpassing the second threshold 1063, the drug delivery device 10 will the calculate a dose of Drug C 1068. In this illustrated profile 1060, this dose of Drug C 1068 equals 19 Units. Although only two offset thresholds are illustrated in this profile 1060, those of skill in the art will recognize alternative threshold arrangements may also be utilized.

Applicants' preferred profiles 1000, 1020, 1040, and 1060 illustrated in FIGS. 45-48 offer a number of advantages. For example, these illustrated profiles could provide the basis for a single device solution where it is therapeutically desirable to ensure that a patient using the drug delivery device 10 receives a specific, calculated dose of one drug compound in conjunction with the dose they select of another drug compound.

However, the patient would receive such specific, calculated doses of the second compound only once a minimum dose threshold (of a primary drug or Drug A) has been reached or surpassed. As such, these illustrated profiles 1000, 1020, 1040, and 1060 could provide a cost-effective solution where a user's prescribed therapy requires that the primary medicament needs to be titrated up to a minimum value reasonably quickly before it should be taken in combination with a secondary medicament (and perhaps other medicaments), therefore rendering at least a two device option more costly and/or wasteful. Such a two device option may be more costly and/or wasteful as the device containing Drug A may be only part utilized at the point where the patient switches to the combination product.

An additional benefit stems from the situation that patients are sometimes required to carry out a priming step with their drug delivery device. Such a priming step may be required either prior to a first use of the drug delivery device or perhaps prior to each time a dose is to be administered by the drug delivery device. In the example of pen type drug delivery devices, one of the principle reasons for the set up prime is to remove clearances/backlash in the mechanism, thereby helping ensure that the first dose delivered is within the required dose accuracy range. The in-use prime (sometimes referred to in certain relevant art and/or literature as a “safety shot”) is recommended for some pen type drug delivery devices. For example, such a safety shot may be recommended so as to confirm that the dose setting mechanism within the device is functioning properly. Such a safety shot is also often recommended so as to confirm that the delivered dose is accurately controlled and also to ensure that the attached dose dispenser (e.g., double ended needle assembly) is not blocked. Certain safety shots also allow the user to remove air from the dose dispenser prior to a user setting and therefore administering a dose. For a multi primary pack device, a profile of this type would enable the ‘in use safety’ prime to be undertaken using primary medicament only, thereby minimizing potential wastage of the secondary medicament. For example, a particular combination therapy where this type of profile might be particularly appropriate is for the combined delivery of a long acting insulin or insulin analog along with a GLP-1 or a GLP-1 analog for early-stage diabetics. For example, there is a reasonably large variation in the size of the insulin doses across patient population, whereas GLP1 doses are broadly constant (except during the titration phase where is generally increases in stepped intervals) across the patient population. For this particular type of combination therapy, titration of the GLP1 dose is needed during the early stages of treatment. This could be achieved with a combination device through the use different ‘strengths’ of drug within the GLP1 cartridge or reservoir (e.g., using 10, 15 or 20 g per 0.2 ml concentrations for instance). The proposed delivery profiles illustrated in FIGS. 45-48 would enable the user to perform a safety shot of the long acting insulin only without wasting GLP1. In this example the accuracy of the insulin dose is the more important than the accuracy of the GLP1 dose which is why performing the safety shot with insulin only is preferred.

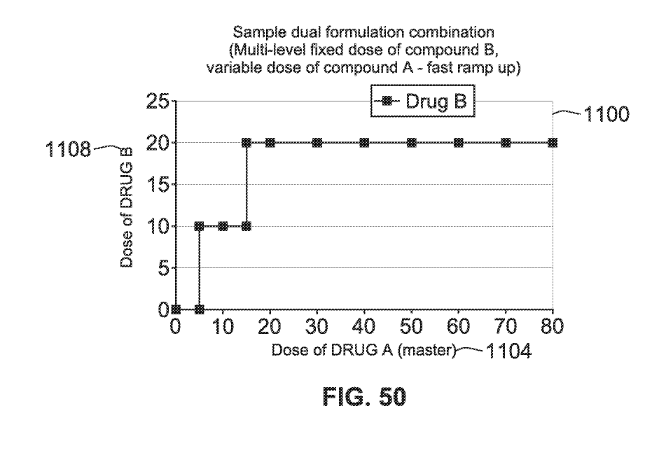

As previously described, the delivery of combination drug products (i.e., single doses that are made up from the combination of two or more individual drug formulations) in a format where the delivered dose profile is predefined, offers a number of key benefits for both a patient and the treatment of a particular condition. For certain therapies it might be beneficial for the dose of the secondary medicament to increase in fixed stepped increments as the corresponding dose of primary medicament increases, but for each of these stepped increases to only occur once a specific predefined threshold dose of primary medicament has been exceeded. The relative ‘spacing’ between these threshold values of the primary medicament may or may not be regular. Again, such profiles of this type are not achievable from a combination drug that is co-formulated into a single primary pack (such as, but not limited to, a standard 3 ml glass cartridge) where the concentration of the various constituent parts is constant. Two exemplary profiles 1080 and 1100 are illustrated in FIGS. 49 and 50, respectively.

For example, FIG. 49 illustrates an arrangement of a predefined multi-level fixed dose-variable dose therapeutic profile 1080 that comprises a slow ramp up and that may be programmed into the drug delivery device 10. Specifically, FIG. 49 illustrates a sample dual formulation having a multi-level fixed dose of Drug B 1088 and having a variable dose of Drug A 1084 and a slow ramp up.

This particular delivery profile could provide the basis for a single device solution where it is therapeutically desirable for the dose of the secondary medicament to increase in a stepped (rather than linear) manner as the dose of primary medicament is increased. This may be related to the specific safety and efficacy characteristics of a prescribed therapy, or situations where titration of the secondary medicament is stepped, as is the case for the injection of GLP1 type drugs (for the treatment of early stage, Type II diabetes).

FIG. 50 illustrates an alternative profile 1100 for defining a predefined multi-level fixed dose-variable dose therapeutic and that may be programmed into the drug delivery device 10. As illustrated, this particular predefined multi-level fixed dose-variable dose therapeutic profile comprises a quick ramp up. In this preferred profile 1100, Applicants' propose a multi-level fixed dose of Drug B 1108 and a variable dose of Drug A 1104 profile. In this case, the profile 1100 describes the delivery of stepped fixed doses of Drug B once corresponding threshold doses of Drug A have been exceeded. The illustrated profiles in FIGS. 49 and 50 have certain potential benefits in terms of splitting a set and calculated combined dose. In addition to the previously discussed advantages, it has been acknowledged that users of drug delivery devices (such as pen type drug delivery devices) may sometimes split their target dose into two, smaller doses. This may occur as a patient transitions from a device that is nearly empty to a replacement device, or because the delivery of a ‘large’ dose as a singular event is problematic (even painful). For single formulation devices, or combination device where the various constituent elements are delivered in a fixed ratio to each other, splitting a dose into smaller parts does not affect the dose that is ultimately received. However, for combination devices where a patient receives a fixed dose of one medicament irrespective of the selected dose of the primary medicament as previously described, splitting a dose could result in an overdose of one of the individual medicaments. The careful utilization of this type of multi-level profile, however, can provide a reasonably robust solution to this particular user scenario.

As just one example, consider a patient who generally takes between 50 and 80 units of Drug A (e.g., an insulin or insulin analog), and whose target dose of Drug B (e.g., a GLP-1 or GLP-1 analog) is 20 units. Assuming that the patient has been prescribed with a device utilizing the therapeutic profile detailed in FIG. 49, then their target prescription would be achieved if each dose is administered as a single injection. This would not be the case where the patient decides to split their target dose into two smaller doses. In an example embodiment, the device may determine that the two subsequent injections are split injections of a single target dose, for example by determining that a cartridge of one of the medicaments was changed, or by determining that only a small amount of time has passed since the last injection, for example less than 30 minutes. Referring to the profile of FIG. 49, a patient may want to administer a dose of 50 units of drug A. The device would determine that a dose of 10 units of drug B corresponds to a dose of 50 units of drug A. However, in a first injection, 25 units of drug A are selected, for example as the cartridge only contains a remainder of 25 units. The device determines according to the profile 10 units of drug B. 5 minutes later (for example after exchanging the cartridge) another 25 units of drug A are selected. As the time since the last injection is less than the threshold of 30 minutes, the device determines that the new selection of 25 units is a second dose of a split dose of drug A of 50 units. Therefore, the device determines the dose of drug B for the second injection to be 0 units, as 50 units of drug A will result in 10 units of drug B according to profile 1080, and as 10 units of drug B have already been administered in the first injection of the split dose.

Applicants' electro-mechanical dose setting mechanism is of particular benefit where a targeted therapeutic response can be optimized for a specific target patient group. This may be achieved by a microprocessor based drug delivery device that is programmed to control, define, and/or optimize at least one therapeutic dose profile. A plurality of potential dose profiles may be stored in a memory device operatively coupled to the microprocessor. For example, such stored therapeutic dose profiles may include, but are not limited to, a linear dose profile; a non-linear dose profile; a fixed ratio fixed dose profile; a fixed dose variable dose profile; a delayed fixed dose variable dose profile; or a multi-level, fixed dose variable dose profile as discussed and described in greater detail below. Alternatively, only one dose profile would be stored in a memory device operatively coupled to the microprocessor. In one dual medicament drug delivery device arrangement, the dose of the second medicament may be determined by way of a first therapeutic profile such as those identified above. In one drug delivery device comprising three medicaments, the dose of the second medicament may be determined by way of a first therapeutic profile while the dose of the third medicament may be determined by either the same first therapeutic profile or a second different therapeutic profile. As those of ordinary skill in the art will recognize, alternative therapeutic profile arrangements may also be used.

### B. Medicated Module

As noted above, the drug delivery system disclosed herein includes two major components: an auto-injector device (as described in detail above) that contains at least two medicaments (e.g., a first and a second medicament) and a medicated module (which is described in detail below) that contains at least one medicament (e.g., a third medicament). The medicated module interfaces with the auto-injector device such that a combination dose of all the medicaments can be delivered via a single dispense interface of the medicated module when the system is activated (e.g., the delivery button on the auto-injector device is actuated).

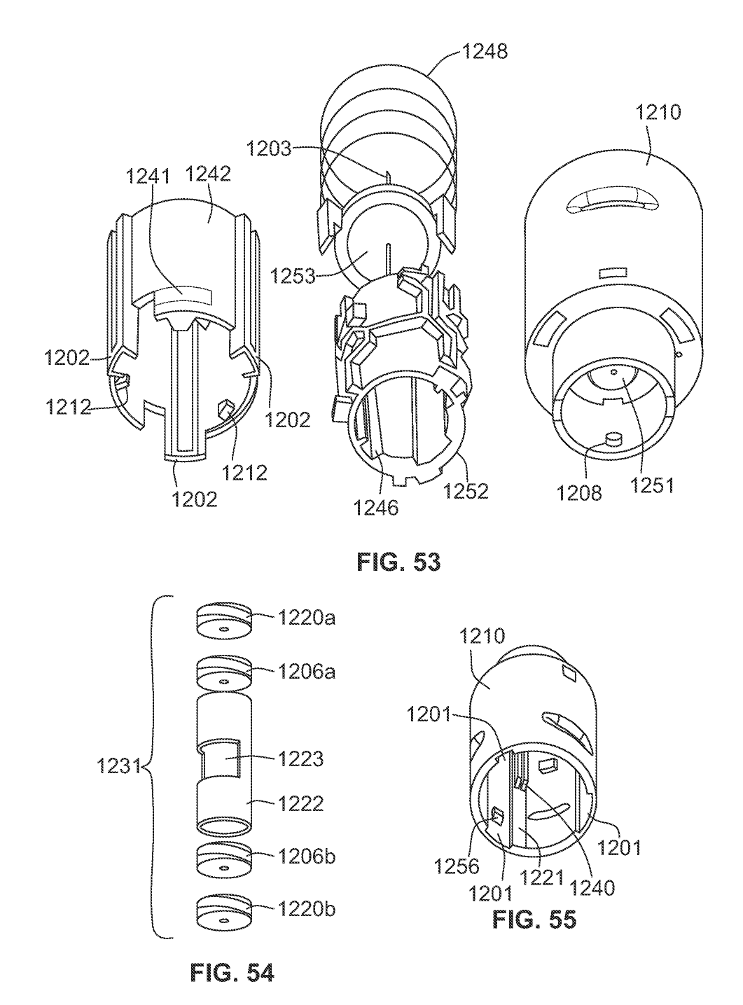

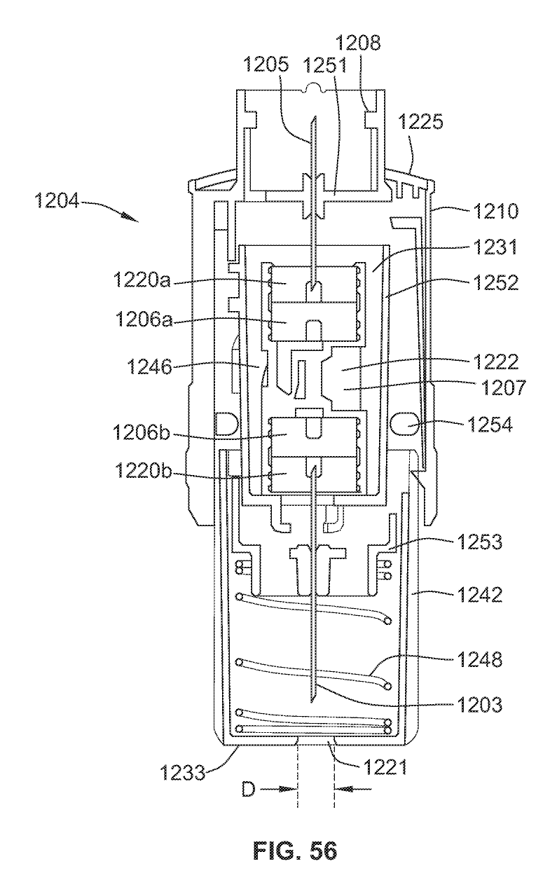

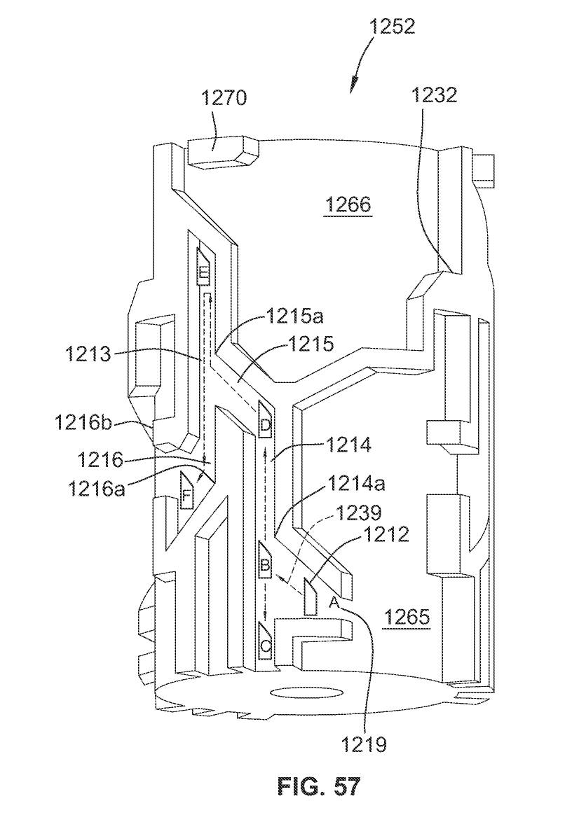

Each medicated module is preferably self-contained and provided as a sealed and sterile disposable module that has a connecting means 1208 compatible with the connecting means/hub 216 of the interface 200 of the auto-injector device 10. Although not shown, the medicated module 1204 could be supplied by a manufacturer in a protective and sterile container, where the user would peel or rip open a seal or the container itself to gain access to the sterile medicated module. In some instances it might be desirable to provide two or more seals for each end of the medicated module. Although connecting means 216 on interface 200 of the auto-injector device 10 is shown as threads, any known connecting means can be used to attach the medicated module 1204 to the device 10, including all types of permanent and removable connection means, such as threads, snap locks, snap fits, luer locks, bayonet, snap rings, keyed slots, and combinations of such connections. For instance, FIGS. 53, and 56 illustrate the connecting means 1208 of the medicated module as a unique bayonet type connection. Accordingly, the interface 200 that connects the auto-injector 10 to the medicated module 1204 would need to include a corresponding byonet type connection.

The examples of the medicated module 1204 described herein have the benefit of the medicament 1207 being a single dose being contained entirely within capsule 1231 (see FIG. 56), and specifically in reservoir 1222, hence minimizing the risk of material incompatibility between the medicament 1207 and the materials used in the construction of the medicated module 1204, specifically housing 1210, inner housing 1252, or any of the other parts used in the construction of the medicated module. To minimize the residual volume of the medicament 1207, caused by recirculation and/or stagnant zones, that might remain in capsule 1231 at the end of the dispense operation, it is preferable to have a flow distributor 1223 as an integral part of reservoir 1222 (see FIG. 54). The reservoir 1222 containing the single dose of the medicament 1207 can be sealed with septa 1206a and 1206b, which are fixed to the capsule using keepers or plugs 1220a and 1220b. Preferably the keepers have fluid channels that are in fluid communication with needles 1203 and 1205 and with bypass 1246, which is preferably part of the inside surface of bypass housing 1252. Together this fluid path allows priming of the auto-injector drug delivery device 10 before injection. Preferably the reservoir, flow distributor, keepers, and bypass can be made from materials that are compatible with the medicaments 92, 102 contained in the cartridges/reservoirs 90, 100 of the auto-injector 10. Examples of compatible materials of construction include, but are not limited to, COC (an amorphous polymer based on ethylene and norbonene, also referred to as cyclic olefin copolymer, ethylene copolymer, cyclic olefin polymer, or ethylene-norbornene copolymer); LCP (a liquid crystal polymer having an aramid chemical structure that includes linearly substituted aromatic rings linked by amide groups, and further can include partially crystalline aromatic polyesters based on p-hydroxybenzoic acid and related monomers and also highly aromatic polyesters); PBT (polybutylene terephthalate thermoplastic crystalline polymer or polyester); COP (a cyclic olefin polymer based on ring-opening polymerization of norbornene or norbornene-derivatives); HDPE (high density polyethylene); and SMMA (styrene methyl methacrylate copolymer based on methyl methacrylate and styrene). The needle pierceable septa, bungs, and/or seals that are used with both the capsule and the primary medicament cartridge can be manufactured using TPE (thermo plastic elastomer); LSR (liquid silicone rubber); LDPE (low density polyethylene); and/or any kind of medical grade rubber, natural or synthetic.

The design of flow distributor 1223 should ensure that at least about 80% of the medicament 1207 contained in the medicament module 1204 is expelled from reservoir 1222 through the distal end of needle 1203. Preferably at least about 90% should be expelled. Ideally, displacement of the first and second medicaments 92, 102, from the auto-injector 10, through the capsule 1231 of the medicated module, 1204 will displace the single dose of the medicament 1207 stored in reservoir 1222 without substantial mixing of the first/second medicaments 92, 102 with medicament 1207.

Attachment of the medicated module 1204 to the auto-injector device 10 causes proximal needle 1205 to penetrate septum 270 of the interface 200 that is connected to the distal end of the auto-injector device 10. Once needle 1205 has passed through the septum 270, fluid communication is made between the first and second medicaments 92, 102 and the needle 1205. At this point, the system can be primed by dialing out a small number of units using dose setting buttons 62, 64 on the control panel 60 of the auto-injector device 10. Once the device 10 is primed, then activation of the needle guard 1242 (i.e., sufficient retraction) allows for the delivery of the medicaments by subcutaneously injecting the medicaments via activation of a dose button 74 on device 10.

One embodiment of the medicated module 1204 is illustrated best in FIGS. 51 and 56. As shown, the medicated module 1204 contains a capsule 1231 comprising a reservoir 1222, two keepers 1220a and 1220b, and two seals 1206a and 1206b. Reservoir 1222 contains a fixed single dose of a medicament 1207. In some cases this medicament 1207 may be a mixture of two or more drug agents that can be the same or different from the primary or secondary medicaments 92, 102 in the drug delivery device 10. Preferably the capsule is permanently fixed within the medicated module, however, in some cases it may be preferred to design the module such that the capsule can be removed when empty and replaced with a new capsule.

As shown in FIGS. 54 and 56, capsule 1231 has ends that are sealed with pierceable membranes or septa 1206a and 1206b that provide a hermetically sealed and sterile reservoir 1222 for the medicament. A primary or proximal engagement needle 1205 can be fixed in hub 1251 connected to the proximal end of housing 1210 of the module 1204 and configured to engage capsule 1231 when needle guard is moved a pre-determined distance in the proximal direction during injection. The outlet, or distal needle 1203, is preferably mounted in lower hub 1253 and initially protrudes into lower keeper 1220b. The proximal end of needle 1203 pierces the lower septum 1206b when the bypass housing 1252 rotates and is moved proximally by the force exerted by needle guard 1242 and spring 1248 during injection.

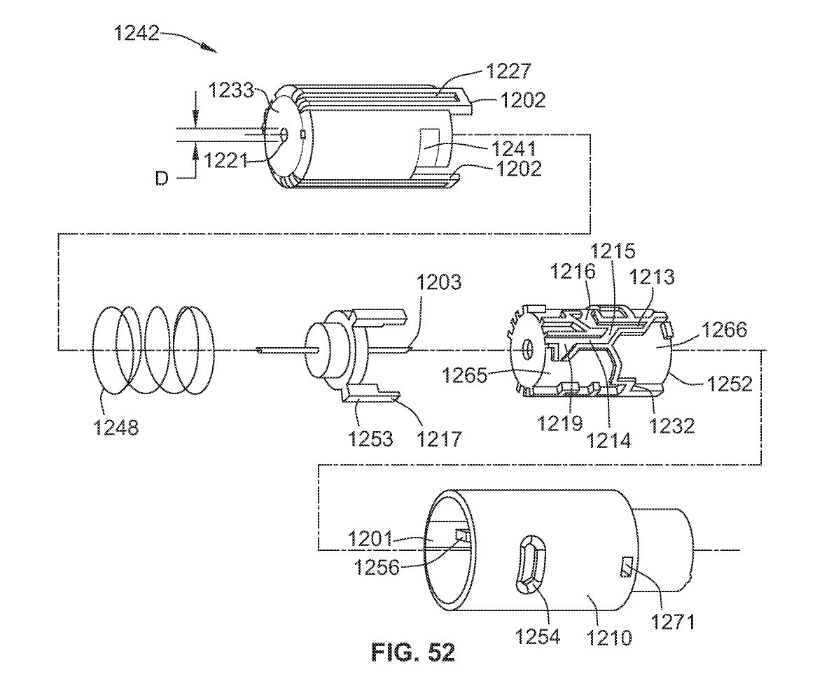

When first attached to the delivery device 10, the medicated module 1204 is set at a pre-use or starting position. Preferably, indicator 1241 shows through window 1254 to inform the user of the pre-use condition of the medicated module. The indicator is preferably a color stripe or band on the outer surface of the proximal end of guard 1242 (see FIG. 52) visible through an aperture in the outer body. The needle guard 1242 is slidably engaged with an inner surface of outer housing 1210 by engagement of arms 1202 and channels 1201 (see FIGS. 53 and 55). Retention snaps 1256 prevent the guard from disengaging the outer housing at its fully extended position. Housing 1210 partially defines an internal cavity 1221 that holds bypass housing 1252, which contains capsule 1231. A portion of the proximal end of housing 1210 defines an upper hub 1251 that holds needle 1205. Optionally, as illustrated in FIG. 56, a shoulder cap 1225 may be added to the proximal outer surface of outer housing 1210. This shoulder cap can be configured to serve as indicia to identify to a user the type/strength of medicament contained in the module. The indicia can be tactile, textual, color, taste or smell.

FIG. 56 shows a cutaway or cross-sectioned view of the medicated module 1204 set in a pre-use or starting state where needles 1203 and 1205 are not piercing septa 1206a and 1206b. In this position, the bypass housing 1252 is at its most extended position and needles 1203 and 1205 are not in fluid communication with medicament contained in capsule 1231. The capsule is supported by bypass housing 1252. In this neutral or suspended state of capsule 1231, the primary and secondary medicaments 92, 102 can flow from their respective cartridges 90, 100 in cartridge holder 40 of device 10, through interface 200, through needle 1205, into keeper 1220a, through bypass 1246, into keeper 1220b, and eventually out needle 1203. This flow configuration allows a user to perform a priming step or procedure by setting a small dose of the primary/secondary medicament 92, 102 using the dose setting buttons 62, 64 on the control panel 60 of the auto-injector device 10.

The compression spring 1248 is positioned between the distal end of bypass housing 1252 and the inner proximal face of guard 1242 (specifically, between the lower hub 1253 and the inner proximal face of guard 1242) to bias the guard 1242 into an extended (guarded) position as illustrated in FIG. 56. Upon assembly, spring 1248 is purposely compressed to supply a proximally directed biasing force against lower hub 1253. This pre-compression of spring 1248 is possible because the lower hub 1253 and the bypass housing 1252 are prevented from moving in an axial proximal direction by radial stand off 1240 located on the inside surface of the outer housing (FIG. 55) that engage with an upper stand off pocket 1266 and legs 1217 of lower hub 1253 engaging lower stand off pocket 1265. The combination of these stand-offs/legs and pockets prevent the lower hub and upper hub needles from piercing into the centre of the capsule until the device is triggered as previously described.

The proximal inside surface of guard 1242 has one or more inwardly protruding features, drive teeth, pips, or like structures 1212 that run in one or more tracks 1213 or guide ways formed in the outer surface of bypass housing 1252. As shown in FIG. 52, track 1213 can be described as four paths, 1219, 1214, 1215, and 1216, that have a specific geometry such that after a single use of the medicated module 1204 the drive tooth 1212 is blocked from further axial movement and the guard (and device) is “locked” in a guarded position where the distal end of the needle is completely and safely covered by guard 1242.

One unique feature of our medicated module 1204 assembly is the user feedback that is given when the assembly is used. In particular, the assembly could emit an audible and/or tactile “click” to indicate to the user that they have firstly triggered the device and secondly reached the “commit” point such that the needle guard will lock safely out upon completion of the injection/removal of the guard from the injection site. This audible and/or tactile feature could work as follows. As mentioned, the needle guard 1242 is rotationally constrained by outer housing 1210 and has one or more drive teeth 1212 that are initially in path 1219 of track 1213 on bypass housing 1252. As the guard is moved proximally, the spring 1248 is further compressed exerting additional force in the proximal direction on lower hub 1253, which is initially constrained axially by the lower stand off pocket 1265 engaged with legs 1217. Likewise, the bypass housing 1252 is constrained from moving proximally by upper stand off pocket stop 1232 engaged with stand off 1240 on the inner surface of outer hosing 1210. The drive teeth 1212 travel in path 1219 causing the bypass housing to rotate slightly. This rotation will disengage the upper stand off 1240 from upper standoff pocket stop 1232, allows the drive teeth to enter path 1214, and unblocks legs 1217 from lower standoff pocket allowing the bypass housing to move proximally carrying with it capsule 1231, where it then can engage needles 1203 and 1205. As the guard continues to move proximally, the drive teeth move from path 1214 passed transition point 1214a into path 1215 causing further rotation of the bypass housing. As this rotation is completed the drive teeth transition to path 1216, potentially emitting an audile “click” sound, as well as a tactile feel, to the user. This transition past point 1215a (and the corresponding point directly below it on the track) constitute the “commit” point and as such, once it has been reached the needle guard 1242 will “lock out” when it extends upon removal of the device from the injection site.

As mentioned, the distal end of the guard 1242 has a planar surface 1233 that provides an added measure of safety and reduces the pressure exerted by the guard on the injection site during an injection. Because the planar surface 1233 substantially covers access to needle 1203 a user is prevented from gaining access to the distal tip of the needle after the assembly is in the locked position. Preferably, the diameter D of needle pass through hole 1221 in the planar surface is no more than 10 times that of the outer diameter of needle cannula 1203.

The outer proximal surface of the needle guard 1242 preferably has indicia 1241 that are preferably at least two different color stripes or bands, each of which is sequentially visible through the opening or window 1254 in outer housing 1210. One color could designate the pre-use or prime state of the module and the other color would indicate that the module is in finished or locked state, another color could be used to denote the transition through the trigger or “commit” point in case a user stops injection after trigger point but before “commit” point. For example, a green color could be the pre-use position and a band of red color could be used to indicate that the module has been used and is locked and an orange color could indicate that the device has been triggered but not locked out. Alternatively, graphics, symbols or text could be used in place of color to provide this visual information/feedback. Alternatively these colors could be displayed using the rotation of the bypass cavity and printed on or embedded into the bypass housing. They could be visible through the aperture by ensuring that the needle guard is made form a transparent material.

FIG. 57 illustrates the travel of drive teeth 1212 in one or more of the paths of track 1213 as illustrated by directional arrow 1239. Drive tooth 1212 begins at position A and through axial movement of the needle guard biases the bypass housing rotationally until it moves past the transition point 1214a and arrives at position B. Once the drive tooth reaches position B the bypass housing and lower needle hub move proximally causing the capsule 1231 to engage needles 1203 and 1205, and the drive tooth moves relatively to position C (this is termed as the triggering of the device) and it is the bypass housing/lower hub moving proximally under the release of stored energy that results in the effective position of the needle guard drive tooth being position C. It is important to note that the needle guard does not move under the action of the release stored energy, it is just the needle hub and the bypass housing that move relatively away from the needle guard at the point of triggering, hence the drive tooth moves from position B to position C. As the needle guard continues to retract, drive tooth 1212 moves proximally in path 1214 to position D, where it exerts a rotational bias on the bypass housing 1252 causing it to rotate again until tooth 1212 passes the transition 1215a (commit point) into path 1216. The drive tooth then moves proximally until position E is reached. At this point, the needle guard 1242 is fully retracted and the full available insertable length of the needle is exposed. Once the user removes the guard from contact with the skin, the guard begins to extend as a result of the distal biasing force exerted by spring 1248 on the inner proximal surface of the guard. The utilization of the stored energy spring to act both as a trigger/piercing spring and also, once extended post triggering, as the needle guard spring is a unique aspect of this design. It negates the need to use two separate springs for these separate functions by locating the spring in a position such that it can fulfill both roles. Initially, for example during assembly or manufacture of the medicated module, the biasing member is compressed exerting a force on the lower hub/bypass housing in preparation for triggering. Once triggered it extends proximally where upon it can then be compressed from the distal end as the needle guard retracts against it. This secondary compression provides the force to push the needle guard back to the extended and locked position as it is removed from the injection site. As the guard moves to its fully extended post-use position, which preferably is less extended than the starting position, the drive tooth 1212 moves distally in path 1216 until it reaches transition point 1216a, where it then rotationally biases the bypass housing 1252 to rotate yet again until tooth 1212 arrives at position F. This last rotation of bypass housing 1252 causes lock out boss 1270 to engage lock out feature 1271. This prevents any further rotational or axial movement of the bypass housing. The needle guard is prevented from further substantial axial movement, as defined earlier, by engagement of the drive tooth with axial stop 1216b. It is within the scope of our invention that a number of tooth arrangements and/or profiles could be used to fulfill the required function described above, e.g., simple equal tooth profiles or more complex multi-angled profiles. The particular profile being dependent upon the required point of commit and rotation of the bypass housing. It is also within the scope of our invention that a similar axial/rotational locking of the lower needle hub to the bypass housing as of the bypass housing to the outer housing, could be integrated to prevent movement of the needle post-triggering and post-lock out.

In any of the above described embodiments of our invention, the medicament 1207 contained in the medicated module may be either in a powdered solid state or any fluid state. The greater concentration of the solid form of the medicament 1207 has the benefit of occupying a smaller volume than the liquid having lower concentration. This in turn reduces the ullage of the medicated module 1204. An additional benefit is that the solid form of the medicament 1207 is potentially more straightforward to seal in the reservoir than a liquid form of the medicament 1207. The device would be used in the same manner as the preferred embodiment with the medicament 1207 being dissolved by the first and/or second medicaments 92, 102 during dispense.

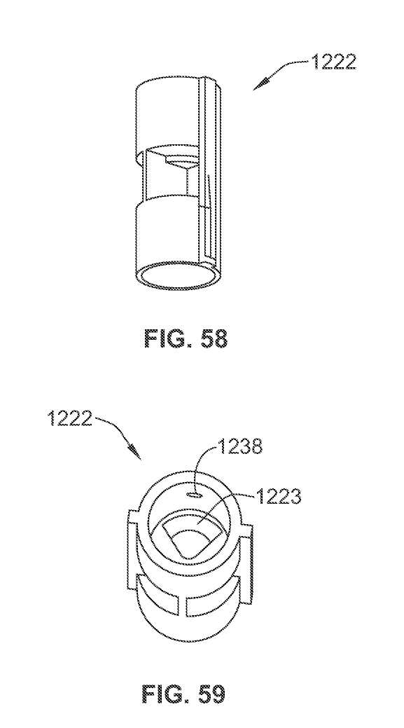

To minimize diffusion of the medicament 1207 contained in the capsule 1231 within the medicated module 1204 into the first and or second medicaments 92, 102 during dispense, the reservoir 1222 has an integral flow distributor 1223. This flow distributor also ensures efficient expulsion of the medicament 1207 from the reservoir 1222 and greatly minimizes residual volume. One possible embodiment of the reservoir 1222 and flow distributor 1223 is illustrated in FIGS. 58 and 59. Preferably the reservoir and flow distributor are manufactured as a single part from materials that are compatible with the medicament 1207 contained therein. A preferred material would be that typically used to manufacture septa or pistons (bungs) found in multi-dose medicament cartridges, although any material that is compatible with the medicament 1207 during long term storage would be equally applicable. The flow distributor 1223 is configured and positioned in reservoir 1222 such that the medicament 1207 fills flow channels that are defined by the shape and location of one or more channels (not shown) inside the reservoir. The shape of the flow channels can be optimized for a plug flow of medicament by varying the dimensions of the flow distributor and/or channels. The cross-sectional area of the annulus formed between the flow distributor and the wall of the reservoir should be kept relatively small. The volume available to store the medicament 1207 would equal the internal volume of the reservoir minus the volume of the flow distributor. Therefore if the volume of the flow distributor is marginally smaller than the internal volume of the capsule, a small volume is left which the medicament occupies. Hence the scale of both the capsule and the flow distributor can be large while storing a small volume of medicament 1207. Resultantly, for small volumes of medicament 1207 (e.g. 50 micro liters), the reservoir 1222 can be of an acceptable size for handling, transport, manufacture, filling and assembly.

Preferably the medicated module 1204 is provided by a drug manufacturer as a stand-alone and separate device that is sealed to preserve sterility. The sterile seal of the module is preferably designed to be opened automatically, e.g. by cutting, tearing or peeling, when the medicated module is advanced or attached to the drug delivery device by the user. Features such as angled surfaces on the end of the injection device or features inside the module may assist this opening of the seal.

The medicated module of 1204 is designed to operate in conjunction with various examples of the auto-injector device 10 described above, Although the examples of the medicated module are described as containing a single medicament, it should be understood that the medicated module may contain more than one medicament.

Further, a series of medicated modules containing the same or different medicaments may be used in conjunction with any of the exemplary auto-injector devices described above.

Exemplary embodiments of the present invention have been described. Those skilled in the art will understand, however, that changes and modifications may be made to these embodiments without departing from the true scope and spirit of the present invention, which is defined by the claims.

### LIST OF REFERENCES

1 drug delivery system
10 auto-injector drug delivery device
14 main body
15 distal end
16 proximal end
18 end cap
20 outer surface
40 cartridge holder
42 distal end
46 first window
47 second window
48 outwardly protruding member
50 cartridge retainer
52 cartridge retainer
60 control panel region
62 first dose setting button
64 second dose setting button
66 OK button
70 detection device
74 injection/delivery button
80 digital display
82 first display region
86 second display region
90 first cartridge/reservoir
92 primary/first medicament
94 stopper
100 second cartridge/reservoir
102 secondary/second medicament
104 stopper
110 cartridge identification system
116 cartridge retainer
118 cartridge holder
120 cartridge
122 label
124 bar code
126 bar code reader
128 light source
130 photo diode
200 interface hub
210 main outer body
212 main outer body
213a first rib
213b second rib
214 distal end
215 inner surface
216 needle hub
217 first recess
218 extending wall
219 second recess
220 first inner body
222 outer surface
224a cooperating grooves
224b cooperating grooves
226 proximal surface
230 second inner body
231 cavity
240 first proximal piercing needle
244 proximal piercing end portion
250 second proximal piercing needle
254 piercing end portion
260 valve seal
262 first non-return valve
264 first fluid groove
266 second fluid groove
268 second non-return valve
270 septum
280 holding chamber
290 outlet port
300 control unit
302 microcontroller
304 power management module
306 battery
308 battery charger
310 USB connector
312 USB interface
314 Bluetooth interface
316 switches
318 push buttons
300 control unit
320 real time clock
322 digital display module
324 memory device
326 first optical reader
328 second optical reader
330 sounder
332 first motor driver
334 second motor driver
336 first motor
338 second motor
350 printed circuit board assembly
500 drive train/electro-mechanical drive unit
502 independent mechanical driver
506 independent mechanical driver
510 battery
514 first telescoping piston rod
516 piston rod
517 external thread
518 proximal portion
519 distal portion
520 printed circuit board assembly
521 distal end
522 first motion detection system
524 first motor pinion
526 rotating gearing portion
528a first flag
528b second flag
530 first motor
531 output shaft
532 connector
534 digital encoder
536 motor
540 first gearing arrangement
544 second motion detection system
600 alternative drive train arrangement/electro-mechanical drive unit
602 independent mechanical driver
606 independent mechanical driver
610 battery
614 telescoping piston rod
616 telescoping piston rod
618 proximal portion
620 printed circuit board assembly
622 distal portion
623 distal end
630 first motor
632 connector
636 second motor
637 shaft
638 connector
640 first gearing arrangement
643 motor pinion
644 telescope plunger
645 threaded section
646 second gearing arrangement
647 key
652 compound reduction gear
654 compound reduction gear
656 compound reduction gear
660 nested piston rod
670 transfer shaft
680 input screw
681 integrated geared part
682 threaded section
700 potential deliverable therapy
702 primary medicament
704 secondary medicament
706 area
710 area
712 lower limit
714 upper limit
720 potential delivered therapy
724 compound A
726 compound B
730 minimum value
732 maximum value
740 minimum value
742 maximum value
744 overall maximum value
760 predefined therapeutic profile
764 compound A
766 compound B
780 therapeutic profile
782 Drug A
784 Drug B
786 Drug C
800 therapeutic profile
802 Drug A
804 Drug B
806 Drug C
808 Drug D
820 therapeutic profile
824 Drug A
825 maximum dose
826 minimum dose
828 Drug B
860 proposed therapy profile
864 Drug A
868 Drug B
880 exemplary profile
884 Drug A
886 Drug B
888 Drug C
900 exemplary profile
904 Drug A
906 compound B
920 exemplary profile
924 Drug A
926 Drug B
928 Drug C
940 low dose threshold therapeutic profile
944 compound A
948 compound B
950 low dose threshold therapeutic profile
952 compound A
958 compound B
960 low dose threshold therapeutic profile
966 Drug B
968 Drug C
980 variable dose therapeutic profile
982 compound A
986 compound B
990 variable dose therapeutic profile
992 Drug A
994 Drug B
996 Drug C
1000 variable dose therapeutic profile
1004 Drug A
1006 low dose threshold
1008 Drug B
1020 variable dose therapeutic profile
1024 Drug A
1026 high threshold
1028 compound B
1040 variable dose therapeutic profile
1042 low threshold
1044 Drug A
1046 Drug B
1048 Drug C
1060 variable dose therapeutic profile
1062 offset threshold
1063 offset threshold
1064 Drug A
1066 Drug B
1068 Drug C
1080 exemplary profile
1084 Drug A
1088 Drug B
1100 exemplary profile
1104 Drug A
1108 Drug B
1201 channels
1202 engagement arms
1203 distal needle/dispense interface
1204 medicated module
1205 proximal needle
1206a top septum/membrane/seal
1206b bottom septum/membrane/seal
1207 medicament in medicated module
1208 attachment means/connector
1210 housing
1212 drive tooth
1213 track
1214 path
1214a transition point
1215 path
1215a transition point
1216 path
1216a transition point
1216b axial stop
1217 legs
1219 path
1220a keeper
1220b keeper
1221 hole
1222 reservoir
1223 flow distributor
1225 shoulder cap
1231 capsule
1233 planar surface
1239 path/directional arrow
1240 radial stand off
1242 guard
1246 bypass
1248 spring/biasing member
1251 upper hub
1252 bypass housing
1253 lower hub
1254 window
1256 retention snap
1265 lower stand off pocket
1266 upper stand off pocket
1270 lock out boss
1271 lock out feature
1232 upper stand off pocket stop

1. A drug delivery system for delivering at least three medicaments, the drug delivery system comprising:
(a) an auto-injector device configured to deliver at least one dose of at least a first and a second medicament, the auto-injector device comprising:
(i) a control unit,
(ii) an electro-mechanical drive unit operably coupled to the control unit, the electro-mechanical drive unit also coupled to a first reservoir and a second reservoir containing the first and second medicaments respectively
(iii) an operator interface in communication with the control unit, and
(iv) an interface hub configured for fluid communication with the first and second reservoirs,
wherein activation of the operator interface sets a dose of the first medicament and based on the set dose of the first medicament, the control unit determines a dose of the second medicament based at least in part on a therapeutic dose profile; and
(b) a medicated module attached to the interface hub of the auto-injector device, the medicated module comprising:
(i) an outer housing having an inner surface, a proximal end, and a distal end, wherein the proximal end includes an upper hub holding a first double-ended needle, and wherein the proximal end is connected to the interface hub of the auto-injector drug delivery device,
(ii) a bypass housing having an outer surface and slidably engaged with an upper radial stand off on the inner surface of the outer housing,
(iii) a reservoir within the bypass housing containing a single dose of a third medicament,
(iv) a guard having an internal proximal face and a drive tooth on an inner surface, where the drive tooth is slidably engaged with a track on the outer surface of the bypass housing,
(v) a lower hub slidably engaged with the outer surface of the bypass housing and slidably engaged with the inner surface of the guard, wherein the lower hub holds a second double-ended needle, and
(vi) a biasing member engaged between the internal proximal face of the guard and the lower hub,
wherein the guard is movable between a distal and a proximal position, and wherein movement of the guard in proximal direction causes the bypass housing to move in a proximal direction and causes the reservoir to come into fluid communication with the first and second double ended needles.

2. The system of claim 1, wherein activation of the operator interface of the auto-injector device causes the electro-mechanical drive unit to dispense the dose of the first medicament and the dose of the second medicament through the interface hub and through the reservoir of the medicated module, thereby forcing the third medicament out of the reservoir.

3. The system of claim 1, wherein the first and second reservoirs comprise multi-dose cartridges having a stopper and a pierceable septum.

4. The system of claim 1, wherein the biasing member of the medicated module comprises a spring.

5. The system of claim 1, wherein the biasing member of the medicated module exerts a force on the lower hub when the guard is pushed in a proximal direction causes the bypass housing to move in a proximal direction.

6. The system of claim 1, wherein the track on the outer surface of the bypass housing of the medicated module comprises a first, second, third, and fourth path.

7. The system of claim 6, wherein the guard of the medicated module is always rotationally constrained by the outer housing, wherein the bypass housing is rotationally constrained when the drive tooth is in the second path of the track, wherein the bypass housing is rotationally constrained when the drive tooth is in at least a portion of the fourth path of the track), and wherein the medicated module provides an audible or tactile indication to a user when the bypass housing rotates as the drive tooth moves from the second path to the fourth path due to proximal movement of the guard.

8. The system of claim 1, wherein the interface hub of the auto-injector device and the medicated module include corresponding exclusive attachment features.

9. The system of claim 1, wherein a pre-defined amount of needle guard retraction places all three medicaments in fluid communication with the dispense interface.

10. The system of claim 1, wherein the auto-injector device contains a first cartridge containing a long acting insulin and a second cartridge containing a short acting insulin, and the reservoir of the medicated module contains a GLP-1.

11. The system of claim 1, wherein the bypass housing of the medicated module further comprises a fluid flow path or bypass around the reservoir, wherein the proximal needle and the distal needle are in fluid communication with the fluid flow path or bypass.

12. The system of claim 1 having a priming state and an injection state, wherein in the priming state the system is configured to allow at least one of the medicaments contained in the auto-injector device to be expelled through the dispense interface and in the injection state the system is configured to allow all the medicaments of the system to be expelled through the dispense interface.

13. The system of claim 12, wherein in the priming state the medicated module is in a pre-use or starting state where the needles and are not in fluid communication with the medicament of the medicated module and wherein in the injection state the medicated module is in a ready-to-use or combination dose state where the needles and are in fluid engagement with the medicament of the medicated module.

14. A preparation method for delivering a combination of medicaments, the method comprising:
attaching an interface hub to a distal end of an auto-injector device that contains medicament;
attaching a medicated module containing a third medicament, and including a proximal and a distal needle, to a distal end of the interface hub such that the proximal needle of the medicated module is in fluid communication with both the primary and secondary medicaments;
setting a desired dose of the main medicament using a dose setter of the auto-injector device;
pressing a needle guard of the medicated module against the skin of a user such that the needle guard retracts, thereby placing the distal needle of the medicated module in fluid communication with the medicaments.

15. A drug delivery system for delivering a combination of medicaments and/or fluids, the drug delivery system comprising:
an auto-injector device comprising
(i) a dose setting mechanism,
(ii) a first cartridge containing a first medicament,
(iii) an interface hub including an outlet port that is in fluid communication with the cartridge(s), and
(iv) a delivery button; and
a medicated module attached to a distal end of the interface hub of the auto-injector device, wherein the medicated module includes
(i) a reservoir containing a medicament or fluid,
(ii) a proximal needle,
(iii) a distal needle, and
(iv) a slidable needle guard,
wherein a pre-defined amount of proximal movement of the needle guard places the distal needle in fluid communication with the first medicament contained in the auto-injector drug delivery device and in fluid communication with the reservoir within the medicated module,
wherein a single actuation of the delivery button of the auto-injector device causes a predefined combination dose of medicaments to be delivered via the distal needle of the medicated module, and
wherein, during delivery, the first medicament contained in the auto-injector flows through the reservoir of the medicated module, thereby forcing the contents out of the reservoir.

16. The system of claim 15, wherein the auto-injector device further comprises a second cartridge containing a second medicament, wherein the interface hub comprises a first and a second proximal needle, wherein the first and second proximal needles are in fluid communication with the first and second cartridges respectively, wherein the single actuation of the delivery button causes a predefined dose of the second medicament to be delivered via the distal needle of the medicated module with the predefined combination dose of medicaments.

17. The system of claim 15, wherein the first medicament comprises an insulin or insulin analog.

18. The system of claim 15, wherein the second medicament comprises a GLP-1 or a GLP-1 analog.

19. The system of claim 16, wherein the auto-injector device further comprising:
(i) a control unit,
(ii) an electro-mechanical drive unit operably coupled to the control unit, the electro-mechanical drive unit also coupled to the first cartridge and the second cartridge containing the first and second medicaments respectively,
(iii) an operator interface in communication with the control unit,
wherein activation of the operator interface sets a dose of the first medicament and based on the set dose of the first medicament, the control unit determines a dose of the second medicament based at least in part on a therapeutic dose profile.
